(19) United States
(12) Patent Application Publication
(10) Pub. No.: US 2013/0168228 A1
(43) Pub. Date: Jul. 4, 2013

(54) Photoactive Material Comprising Nanoparticles of at Least Two Photoactive Constituents

(71) Applicants: Geoffrey A. Ozin, Toronto (CA); Engelbert Redel, Steinmauern (DE)

(72) Inventors: Geoffrey A. Ozin, Toronto (CA); Engelbert Redel, Steinmauern (DE)

(21) Appl. No.: 13/821,911

(22) PCT Filed: Sep. 9, 2011
(86) PCT No.: PCT/CA11/01022

§ 371 Date: Mar. 8, 2013

Related U.S. Application Data
(60) Provisional application No. 61/474,495, filed on Apr. 12, 2011.

(51) Int. Cl. B01J 35/00; C07C 29/00; B01J 23/745; B01J 27/224; B01J 23/72; B01J 23/30

(52) U.S. Cl. CPC B01J 35/004; B01J 23/72; B01J 23/30; B01J 23/745; B01J 27/224; C07C 29/00; Y10S 977/773; Y10S 977/811; Y10S 977/775; Y10S 977/902; B82Y 30/00
USPC 204/157.9; 204/157.15; 502/100; 502/300; 502/177; 502/202; 502/240; 502/224; 422/186; 502/345; 502/309; 502/338; 502/331; 502/178; 977/773; 977/811; 977/775; 977/902

(57) ABSTRACT

A photoactive material including nanoparticles of photoactive first and second constituents. The first and second constituents have respective conduction band energies, valence band energies and electronic band gap energies to enable photon-driven generation and separation of charge carriers in each of the first and second constituents by absorption of light in the solar spectrum. The first and second constituents are provided in an alternating layered arrangement of respective first and second layers or are mixed together in a single layer. The nanoparticles have diameters smaller than wavelengths of light in the solar spectrum, to provide optical transparency for absorption of light. The charge carriers, upon photoactivation, are able to participate in redox reactions occurring in the photoactive material. The photoactive material may enable redox reactions of carbon dioxide with at least one of hydrogen and water to produce a fuel.

### CROSS-REFERENCE TO RELATED APPLICATION

The present disclosure claims priority from U.S. provisional patent application No. 61/381,656, filed Sep. 10, 2010, and U.S. provisional patent application No. 61/474,495, filed Apr. 12, 2011, the entireties of which are hereby incorporated by reference.

### FIELD OF TECHNOLOGY

The present disclosure relates to photoactive materials, in particular porous single-layer and porous multi-layered photoactive materials suitable for large-scale applications in generation of fuels from the recycling of carbon dioxide, the splitting of water, as well as environmental air and water purification processes.

### BACKGROUND

As the global demand for energy increases, being exacerbated by the ballooning growth in the world's population, the gap between energy use and carbon dioxide production continues to increase, currently at the rate of about 2 ppmv (parts per million by volume) per year, which corresponds to around 10 billion tons per year of the green house gas carbon dioxide CO₂ released into the earth's atmosphere/troposphere, contributing thereby to global warming.

At current rates of energy usage, it is expected that the world will face a roughly 14 TW energy gap by 2050 which is expected to increase to around 33 TW by 2100.¹ Renewable energy resources like wind, tidal, geothermal, nuclear, biomass, photovoltaic and hydroelectric are unlikely to provide a sufficient amount of energy. By contrast, the sun produces 10×10¹⁵ TW of clean energy that reaches the surface of the earth, of which around 600 TW can be utilized.

There is recognition that environmental pollution and destruction of the ecosystem on a global scale, for example through the incessant use of coal, oil and gas, as well as the long term consequences of allowing this situation to continue unabated with respect to its deleterious effect on global warming may be disastrous. Solutions on a global scale to this global challenge are needed.

The lack of sufficient clean and natural energy sources have drawn much attention and created much concern about the need for ecologically acceptable, chemical technologies, materials and processes to solve this problem.

### SUMMARY

The present disclosure describes a photoactive material. This photoactive material may be provided in a single-layer or multi-layered arrangement, with each layer being a thin, porous, optically transparent layer. The photoactive material may be used as a reactive membrane for heterogeneous gas-solid reactions, in particular the simultaneous reduction of CO₂ and oxidation of H₂O and/or H₂.

Certain embodiments of the disclosed photoactive material may be suitable for large-scale photoreaction applications, such as the industrial-scale production of fuels from the redox reaction of CO₂ and various [H₂]/[H₂O]₁₋ₓ mixtures (where 0≦x≦1), as well as industrial-scale purification of air and/or water, for example as an anti-smog coating or for water-splitting applications. Certain embodiments of the disclosed photoactive material may also be suitable for personal or individual use, for example provided on windows or roofs as a personal renewable energy source.

In some aspects, the present disclosure provides a photoactive material including: nanoparticles of at least one first photoactive constituent; and nanoparticles of at least one second photoactive constituent. The at least one first and second constituents each are selected to have respective conduction band energies, valence band energies and electronic band gap energies, to enable photon-driven generation and separation of charge carriers in each of the at least one first and second constituents by absorption of light in the solar spectrum. The nanoparticles of each of the at least one first and second constituents are mixed together to form a layer. The nanoparticles of each of the at least one first and second constituents have diameters smaller than wavelengths of light in the solar spectrum, to provide optical transparency for absorption of light. The charge carriers, upon photoactivation, are able to participate in redox reactions occurring in the photoactive material.

In some aspects, the present disclosure provides a photoactive material including: nanoparticles of at least one first photoactive constituent; and nanoparticles of at least one second photoactive constituent. The at least one first and second constituents each are selected to have respective conduction band energies, valence band energies and electronic band gap energies, to enable photon-driven generation and separation of charge carriers in each of the at least one first and second constituents by absorption of light in the solar spectrum. The nanoparticles of the at least one first constituent form at least one first layer and the nanoparticles of the at least one second constituent form at least one second layer. The nanoparticles of each of the at least one first and second constituents have diameters smaller than wavelengths of light in the solar spectrum, to provide optical transparency for absorption of light. The photoactive material includes the at least one first layer and the at least one second layer in an alternating layer arrangement. The charge carriers, upon photoactivation, are able to participate in redox reactions occurring in the photoactive material.

In particular, the conduction band and valence band energies of the at least one first constituent may be higher than those of the at least one second constituent, to enable the photon-driven generation and separation of charge carriers. The photon-driven generation and separation of charge carriers may be enabled by absorption of light in the visible spectrum.

At least one layer of the photoactive material may be porous, to permit permeation by reactants and collection of products of the redox reactions.

The photoactive material may allow for redox reactions including the reduction of carbon dioxide and concurrent oxidation of at least one of water and hydrogen into at least one fuel, for example methane and/or methanol.

### BRIEF DESCRIPTION OF THE DRAWINGS

FIGS. 1A and 1B are schematic diagrams of electronic coupling of two example photoactive constituents taking part in a photoreaction in a photoactive material;

FIGS. 2A and 2B are schematic diagram and electron microscope image comparing example multi-layered photoactive materials with the thylakoid membrane ultra-structure of a natural leaf;

FIGS. 3A and 3B are diagrams of photoreactions that may occur in a photoactive material including TiO₂ and CuO photoactive constituents;

FIGS. 4A and 4B are diagrams of photoreactions that may occur in a photoactive material including TiO₂ and Fe₂O₃ photoactive constituents;

FIG. 5 is a schematic diagram of an example multi-layered photoactive material including various additional layers;

FIGS. 6A and 6B show schematic diagrams comparing a photoactive material with a conventional photoactive powder;

FIG. 6C shows a schematic diagram of a photoactive material incorporating various additives;

FIG. 7 shows schematic diagrams of example multi-layered photoactive materials having different multi-layer structures and architectures;

FIG. 8 shows schematic diagrams of example multi-layered photoactive materials having tandem and gradient structures;

FIG. 9 is a schematic diagram of an example photoreactor suitable for incorporating a photoactive material;

FIG. 10 shows an example spectrum illustrating the effects of different light absorption enhancements in a photoactive material;

FIG. 11 shows reflection spectra illustrating examples of the response of photoactive materials having different layer thicknesses;

FIGS. 12A and 12B illustrate the use of photoactive materials on a utility scale in cities and houses, as well as in building integrated photosynthetic units (BIPS);

FIG. 13 is an image of a batch test photoreactor used in an example study of the photoactive material;

FIG. 14 shows a pressure over time graph illustrating results from an example study of the photoactive material;

FIG. 15 shows gas-phase batch gas chromatography measurements from an example study of the photoactive material;

FIGS. 16A and 16B are schematic diagrams illustrating the heterojunction electronic coupling between photoactive nanoparticulate Fe₂O₃/TiO₂ constituents;

FIGS. 17A, 17B and 17C are schematic diagrams illustrating the heterojunction electronic coupling between photoactive nanoparticulate Fe₂O₃/CuO constituents;

FIGS. 18A, 18B and 18C are schematic diagrams illustrating the heterojunction electronic coupling between photoactive nanoparticulate CuO/TiO₂ constituents;

FIGS. 19A and 19B are schematic diagrams illustrating the heterojunction electronic coupling between photoactive nanoparticulate SiC/Cu₂O constituents; and

FIG. 20 shows an electron microscope image of an example of a mixed CuO and Fe₂O₃ nanoparticle single-layer photoactive material.

### DETAILED DESCRIPTION

### Definitions

Throughout the present disclosure, the following terms and definitions are used:

Photoreaction: a chemical reaction that proceeds with the absorption of light (i.e., photons). It can be thought of as a reaction wherein a photon is a reactant.

Photocatalytic reaction: refers to photoreactions in which one photon can react to produce more than one product. For example, A+B+photon (catalyst)→2C.

Photostoichiometric reaction: refers to photoreactions in which one photon can react to produce one product. For example, A+B+photon (catalyst)→C.

Photothermal reaction: refers to reactions in which heat is generated. For example, A+B+photon (catalyst)→C+heat. The generated heat can help to accelerate additional reactions.

Photoactive reaction: refers to photoreactions including photocatalytic, photostoichiometric and photothermal reactions.

Photon-driven: used to describe events resulting from photoreactions. Photons for driving such events may be from natural light, concentrated solar power (CSP) sunlight, or artificial light, for example.

Nanoparticle: for simplicity, throughout this disclosure, this term is used to refer to particles having at least one nanoscale dimension. This term is intended to include, for example, nanospheres, nanocubes, nanopolyhedrons (e.g., nanoicosahedrons, nanooctahedrons, etc.), nanowires, nanorods, nanosheets and any other geometries having at least one nanoscale dimension, including random geometries.

1D periodicity: refers to a multi-layered arrangement that is periodic in the overlapping layers. That is, the layers of the multi-layered structure repeat in a periodic fashion, such as alternating layers.

Electron-hole pair: refers to the presence of an extra electron in one species and the corresponding absence of an electron in a second species. These are charge carriers that are separated from each other, in order to maintain their respective charges.

### Overview

A solution to the current energy and climate problems may be to take a lesson from nature's photosynthetic apparatus, for example leaves with distinct layered and multi-layered membrane architectures (e.g., layered thylakoid stacks comprising the leaf ultra-structure for carrying out photosynthesis) and hierarchical constructions thereof, whereby the leaves of trees and plants, grasses and crops are able to sequester carbon dioxide and water from the atmosphere and in the presence of sunlight convert the mixture into energy-rich carbohydrates, with simultaneous release of oxygen to sustain life on earth.

If a practical solar-driven process could be found for converting carbon dioxide to energy-rich fuels (e.g. methane or methanol) using solar light, with an overall efficiency comparable to or greater than plants, then with just ˜0.2% coverage of the earth's surface, it should be possible to produce 20 TW of energy. This should help to satisfy the global demand with the added advantage of helping to maintain carbon dioxide concentration in the troposphere at today's steady state levels.

In the natural photosynthesis process, light energy is absorbed by “antenna” chlorophyll molecules embedded in the multi-layered cell membranes (referred to as thylakoid membrane stacks) and transferred to reaction center chlorophyll pigments. This light driven reaction requires the cooperation of two different, membrane-bound photochemical assemblies (referred to as photosystems PSI and PSII).² The ability of the photosystems PSI and PSII to preferentially orient themselves in the multi-layer photosynthetic cell membranes of the leafs ultra-structure seems to be a factor for the relatively high efficiency of the photosynthesis process in the natural leaf.² These 1D periodic stacked nanolayered thylakoid stacks have high surface areas, with distinct layer/membrane thicknesses ≈10-12 nm, and are reported to create efficient interaction between incident sunlight and embedded light-harvesting pigments. These thylakoid membrane stacks are also favorable for high efficiency light harvesting processes occurring in natural leaves.³ It would be useful to provide a material that can mimic the function of the natural leaf. Such a material may be referred to as an “artificial leaf”.

The photon-driven conversion of carbon dioxide to fuels, as described above, can be effected by efficient, non-biological, energy conversion photoactive materials, as disclosed herein. Such photoactive materials can be manufactured as coatings, reactors, membranes, panels, tiles or apparatuses to generate fuels through photon-driven reactions. Such fuels, generated through solar power, may be referred to as “solar fuels”.

Fuels that may be generated by the disclosed photoactive materials include and are not limited to the following: hydrogen, carbon monoxide, alkanes (such as methane, ethane, propane and isopropane, linear and branched hydrocarbon isomers and possible mixtures thereof), olefins (such as ethylene, propylene, butylenes and other linear and branched olefin-isomers and possible mixtures thereof), oxygen-rich hydrocarbon compounds (such as methanol, formaldehyde, ethanol, propanol, formic acid, aldehydes and other oxygenated hydrocarbon compounds) as well mixtures thereof. The disclosed photoactive materials are capable of carrying out the reaction to generate such fuels through reaction of sunlight or concentrated solar power (CSP), carbon dioxide, and water and/or hydrogen.

Certain factors should be considered for the realization of practical fuel-forming photoreactions. These factors include one or more of: (i) efficient harvesting of light by strongly light-absorbing photoactive constituents, (ii) efficient creation and separation of charge carriers and (iii) efficient participation of these charge carriers in multi-electron redox reactions, in particular the simultaneous oxidation of water and the reduction of carbon dioxide to fuels, with high activity and selectivity. Furthermore, a practical solar-powered fuel generator may include photoactive materials in the form of a porous single-layer or porous multi-layered photoactive membrane. Such membranes may be designed to control one or more of: (iv) the adsorption, permeability and desorption of gaseous reactant and product streams; (v) the fractionation and condensation of reactants and products; and (vi) the separation of oxygen from organic product fuels.

Photoreaction of Carbon Dioxide and Water and/or Hydrogen

The photoactive materials of the present disclosure are designed to carry out photon-driven conversion of carbon dioxide with water and/or hydrogen to generate fuels. To assist in understanding the present disclosure, this photoreaction is described in further detail.

The photon-driven conversion of CO₂ and various [H₂]ₓ/[H₂O]₁₋ₓ mixtures (wherein 0≦x≦1) into reaction products including one or more fuels (e.g., hydrocarbons, hydrocarbon-containing products, oxygen-rich hydrocarbons, hydrogen, hydrogen-containing products, carbon monoxide, and/or carbon-containing products) can be carried out by the disclosed photoactive materials. Carrying out this photoreaction on a large scale can help to reduce atmospheric CO₂ concentrations on a global scale while providing, on a renewable basis, an energy-dense portable fuel, such as methane or methanol, which would be compatible with the conventional energy and fuel infrastructure.

The foundation of many photoreactions is the generation of electron-hole pairs in the conduction bands (CB) and valence bands (VB), respectively, of a photoactive constituent, such as a metal oxide. The generation of electron-hole pairs is induced by the absorption of photons at least equal in energy to the electronic band gap (Eg) of the photoactive constituent. This is exemplified by the following equation:

photoactive constituent+hν→e_{CB}⁻+h_{VB}⁺

where the photoactive constituent may be, for example, TiO₂, WO₃, ZnO, CuO, Fe₂O₃, SnO₂, antimony tin oxide (ATO)≡Sb:SnO₂, indium time oxide (ITO)≡SiC, ZnS, GaN, CdSe, and mixtures thereof.

The generated negative electron (e⁻) and the positive hole (h⁺) may be used in distinct redox reactions. In general, for photoreactions, the generated electron may be more favorably located on a more basic constituent, while the generated hole may be more favorably located on a more acidic constituent.

The interaction of photoactive constituents may be suitable for heterogeneous gas/solid photoreactions.

Generally, a nanoparticle of a first kind of photoactive constituent in contact with a nanoparticle of a second kind of photoactive constituent can couple electronically. These photoactive constituents are typically metal oxides, although other photoactive constituents may also be used, as will be discussed further below. The electronic interactions of these constituents may be relatively complex.⁴ For example, by coupling unlike constituents, different types of electronic coupling can occur at the interface between adjacent nanoparticles.

The addition of co-catalysts, such as hole and electron scavengers, to photoactive materials may help to sensitize the latter for light-induced redox processes. This will be described in further detail below.

Where two different photoactive constituents are arranged in separate layers that are stacked together in a multi-layered arrangement, electronic coupling between the different photoactive constituents of adjacent photoactive layers can be used to facilitate electron-hole vectorial (i.e., one direction) charge transport and charge carrier separation. Synergistic electronic band gap effects between different photoactive layers leads to improved charge carrier diffusion and separation, suppressing possible charge carrier recombination processes, which will result in higher photoactive performance. This will be discussed in further detail below.

The photoactive materials can include modifications and variations to improve their efficiency in photon-driven conversion of CO₂ to fuels, as will be described in further detail below.

### Photoactive Material

The disclosed photoactive materials include electronically- and chemically-coupled redox-active nanoparticles that carry out the photoreactions described herein.

These nanoparticles typically are nanoparticles of metal oxide constituents (although non-metal oxides and/or other semiconductor materials can also be used, among others) and can be arranged as single layers as well as multi-layered structures. Where the arrangement is a multi-layered structure, the layers can be arranged to have a 1D periodicity.

The disclosed examples of nanoparticle layered photoactive materials with controlled geometry and structure, optical transparency and porosity are useful for redox-based remediation of organic and/or inorganic pollutants (e.g., in water and air), the splitting of water to H₂ and/or O₂, as well as the reduction of carbon dioxide to fuels (e.g., hydrocarbons and oxygen-rich synthetic fuels), under ambient sunlight conditions and/or by using CSP irradiation.

The arrangement of the constituent layers in a multi-layered photoactive material may be periodic or aperiodic, and these layers may be organized to create homo-structures, hetero-structures, gradient structures and/or tandem arrangements.

The photoactive material also displays a controlled degree of porosity, typically ranging from 10-90%, in particular 30-50% by volume. Greater porosity in the material may lead to greater gas and/or liquid permeability and thus greater access of reactants to photoactive nanoparticle surfaces as well as easier collection of products from the photoactive material; on the other hand, less porosity may lead to greater surface area for photoreactions to occur. This trade-off in porosity may be controlled in order to obtain a desired gas diffusion rate, permeability, gas contact time, flow rate, etc. Porosity may also be varied within a single layer or among different layers of the material. Porosity can also be controlled by controlled variations in nanoparticle sizes and/or the layer arrangement.

The photoactive materials may not display any significant losses in their photoactivity after multiple reactions and may furthermore be made of recyclable and reusable constituents.

### Photoactive Constituents

The photoactive material, whether as a single-layer or as a multi-layered arrangement (as described below), includes nanoparticles of at least two photoactive constituents, to carry out the photoreaction of carbon dioxide with water and/or hydrogen to produce fuels. As will be described above, this photoreaction may be enhanced in various ways.

A photoactive constituent may be any species that absorbs photons to generate electrons and/or holes. The photoactive constituent may participate in a photostoichiometric, photocatalytic or photothermal reaction, generally referred to as a photoreaction.

The function and selection of the nanoparticles of photoactive constituents in the photoactive material are described below. Their characteristics and selection thereof are also generally described in the literature'.

Throughout this description, the two different photoactive constituent nanoparticles will be referred to a np(1) and np(2), for simplicity and generalization.

Generally, the nanoparticle size, size distribution, shape, surface characteristics, degree of crystallinity, and optical constants (in particular the refractive index and absorption index) of the constituent nanoparticles are chosen to obtain a desired optical transparency, surface area and porosity in the photoactive material, as will be discussed below. The optical constants can be measured by ellipsometric porosimetry (EP) measurements. The refractive index affects interference of light with the nanoparticle layer while the absorption index affects the strength of absorption of light at energies higher than the electronic band gap of the photoactive constituents.

The layer thickness of the photoactive material (whether the thickness of a single-layer arrangement or the thicknesses of the individual layers in a multi-layered arrangement) is controlled by the manufacturing process, described in greater detail below. In a single-layer photoactive material, where np(1) and np(2) are mixed within the layer, the ratio of np(1) to np(2) is also selected to obtain the desired optical transparency, surface area and porosity.

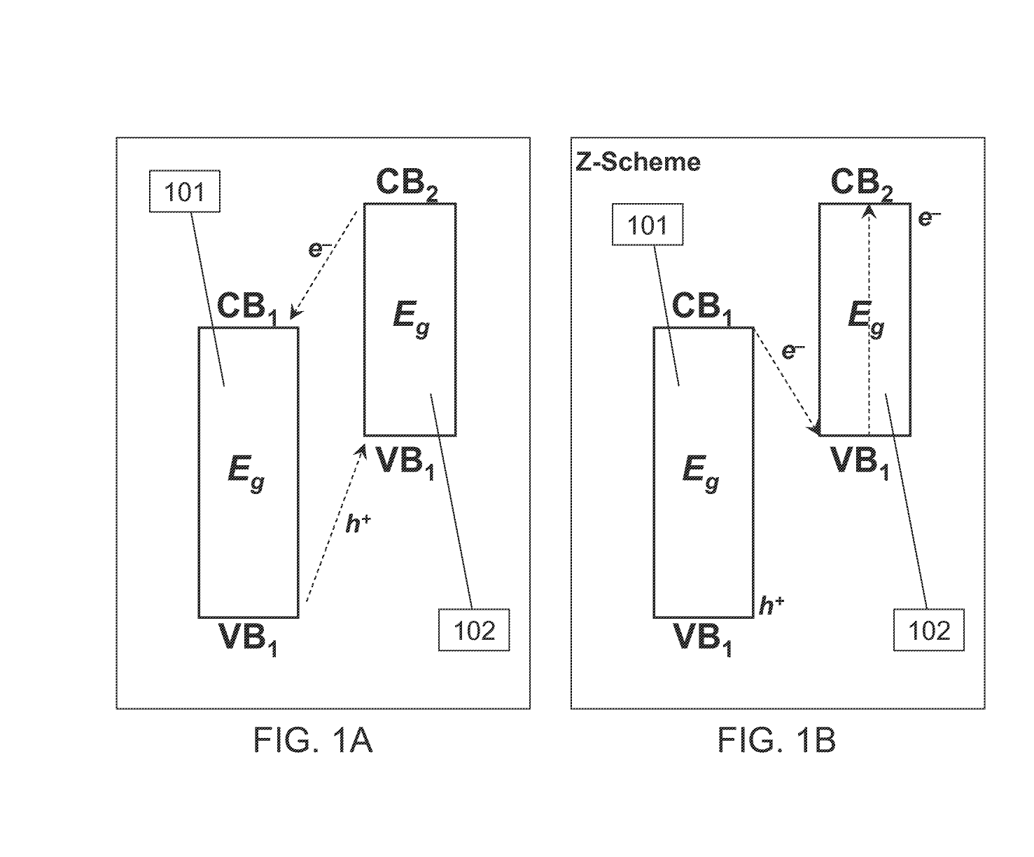

Reference is now made to FIG. 1. In FIG. 1A, np(1) 101 has a simple electronic coupling with np(2) 102. In FIG. 1B, np(1) 101 and np(2) 102 are electronically coupled in a Z-scheme.

The choice of the np(1) and np(2) pairing controls the values of the electronic energies of VB and CB, as well as Eg. The selection of np(1) and np(2) also affects how these values align with respect to each other and positioned with respect to the zero reference energy. Such values are generally known for various species¹³. In the example of FIG. 1, np(1) has lower CB and VB values (shown as CB(1) and VB(1)) than those of np(2) (shown as CB(2) and VB(2)). When np(1) is in contact with np(2), a heterojunction forms at the contact area between the two. The absorption of a photon from incident light results in the generation of electrons and holes in np(1) and np(2). In this example, because CB(2) is higher than CB(1), electrons are transported down the energy gradient from np(2) to np(1), and holes are transported up the energy gradient from np(1) to np(2). The process described generally above results in charge carrier separation of electrons and holes generated in a photo-driven process.

There are many options for aligning the VB and CB energies and selecting Eg to control this vectorial transport of electrons and holes between the two different nanoparticles np(1) and np(2). These values can be measured (e.g., using X-ray photoelectron spectroscopy (XPS)-ultraviolet photoelectron spectroscopy (UPS)) or found in the literature¹³. This kind of electronic band gap engineering is generally known in the semiconductor literature¹³ and can be used to optimize the efficiency of photon-driven generation of electron-hole pairs, vectorially transporting them and separating them effectively to maximize their redox reactions with adsorbed CO₂ and H₂ and/or H₂O. This optimization will help to maximize the rate of production and efficiency of producing fuels in response to incident light. Various methods for optimization of the band gap coupling are known in the art^{6,13}.

Good matching of the CB and VB levels of the photoactive constituents in each layer is useful for realizing a vectorial transfer of charge that is a) from a higher CB to a lower CB and/or b) from a higher CB to a lower VB→CB, which would represent an analogue to the photosynthesis Z-Scheme⁷.

It is generally favorable to have materials with higher and lower CB and VB energy values combined with each other to allow for more efficient charge carrier separation. For example, a high band gap (i.e., having a high Eg value) material (e.g. TiO₂, which has Eg≈3.0-3.2 eV) and a lower band gap (i.e., having a low Eg value) material (e.g., CuO, which has Eg≈1.4-1.6 eV) can be paired. In another example two high band gap materials (e.g. TiO₂/WO₃) can be paired. In another example, two lower or narrower band gap materials (e.g. SiC/CuO) can be paired.

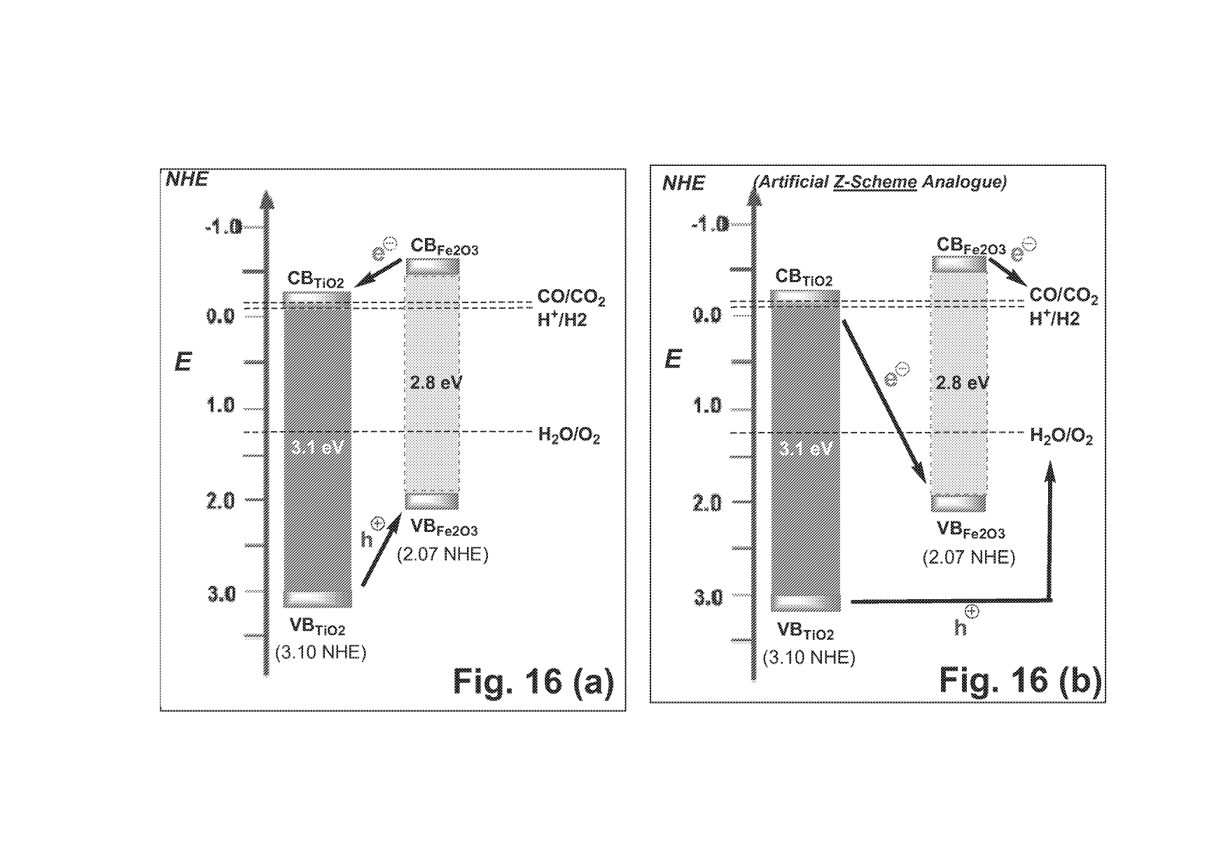

FIGS. 16-19 illustrate electronic coupling in favorable pairings of photoactive constituents. In FIGS. 16-19, the energy scale E is in units of electron volts (eV) using the normal hydrogen electrode (NHE) as a reference.

FIGS. 16A and 16B show the pairing Fe₂O₃/TiO₂, in both simple and Z-scheme electronic coupling, in which the CB of Fe₂O₃ is around −0.5 to −0.7 eV (NHE), which is higher than the CB of TiO₂ which is around −0.15 to −0.35 eV (NHE). Also the VB Fe₂O₃ is around 2.07 eV (NHE), which is higher than the VB of TiO₂ around 3.10 eV (NHE). The difference between the VB and CB is the Eg, in this example around 2.8 eV for Fe₂O₃ and around 3.1 eV for TiO₂.

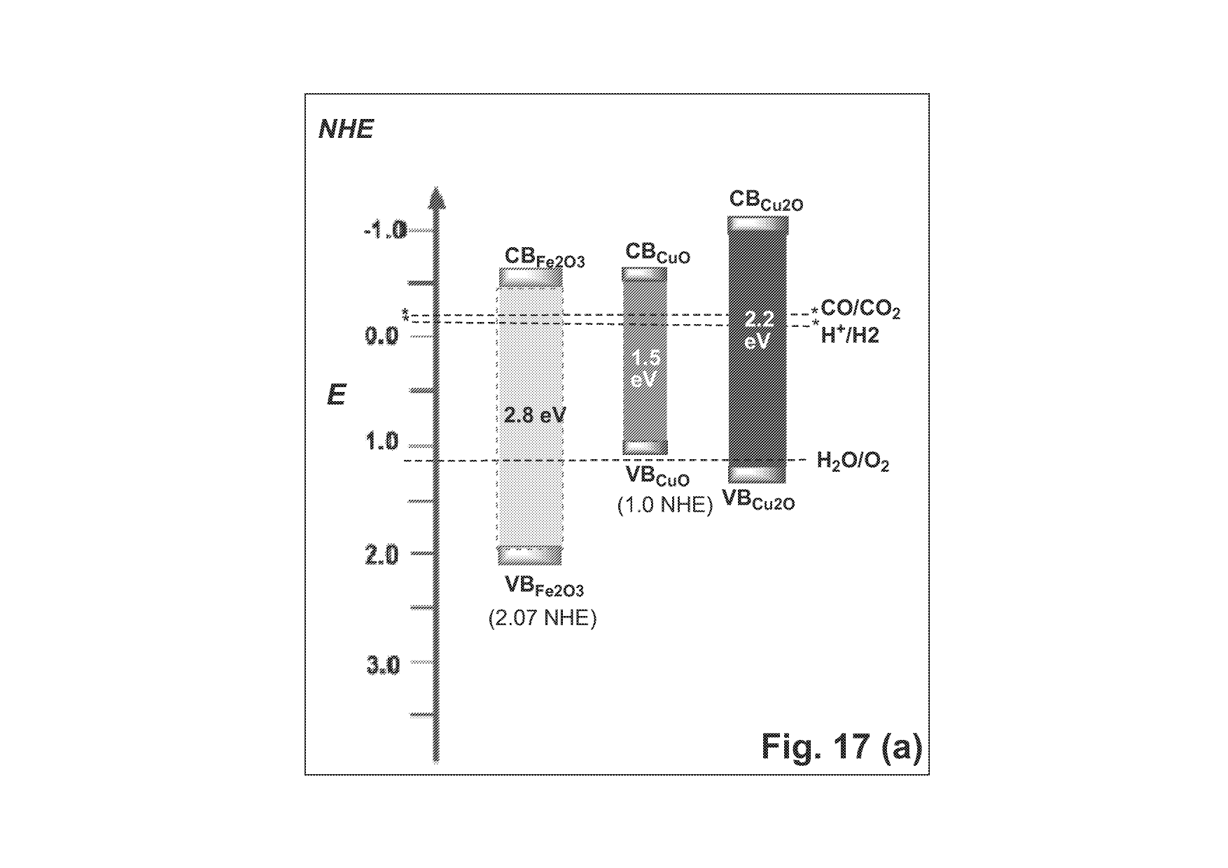

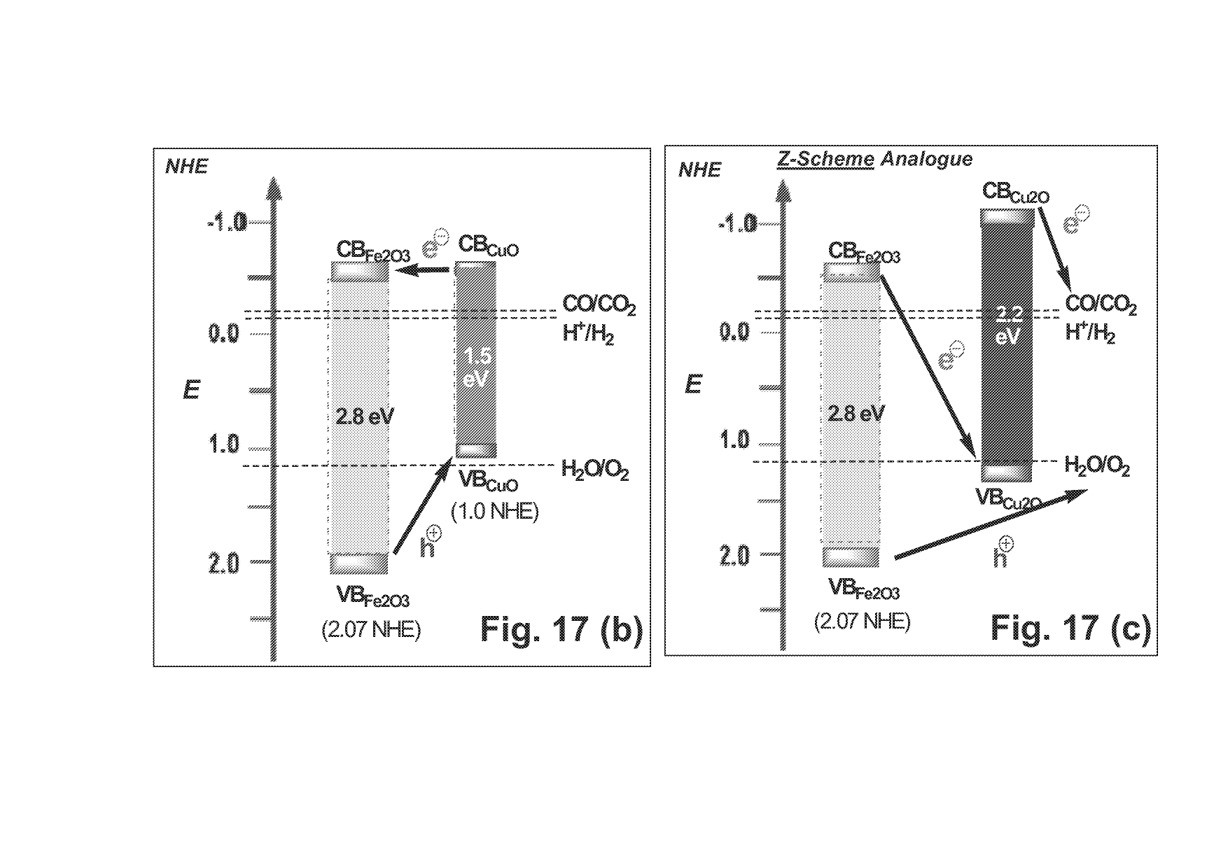

FIGS. 17A-17C show the pairing Fe₂O₃/Cu₂O, in both simple and Z-scheme electronic coupling. In this example, the CB of Fe₂O₃ is around −0.5 to −0.7 eV (NHE), which is lower than the CB of Cu₂O at around −0.9 to 1.1 eV (NHE). Also the VB of Fe₂O₃ is around 2.07 eV (NHE), which is lower than the VB of Cu₂O which is around 1.2 to 1.4 eV (NHE). In this case, the Eg is about 2.2 eV for Cu₂O.

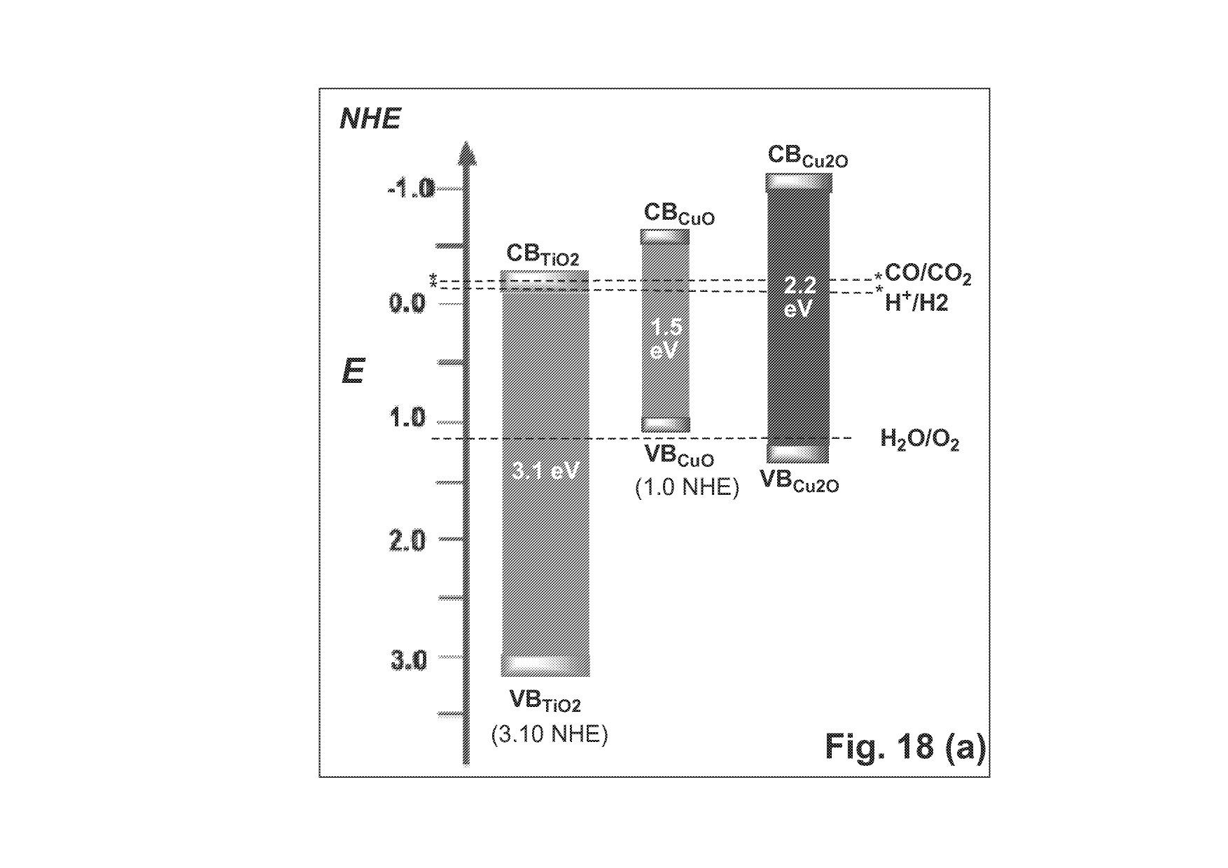

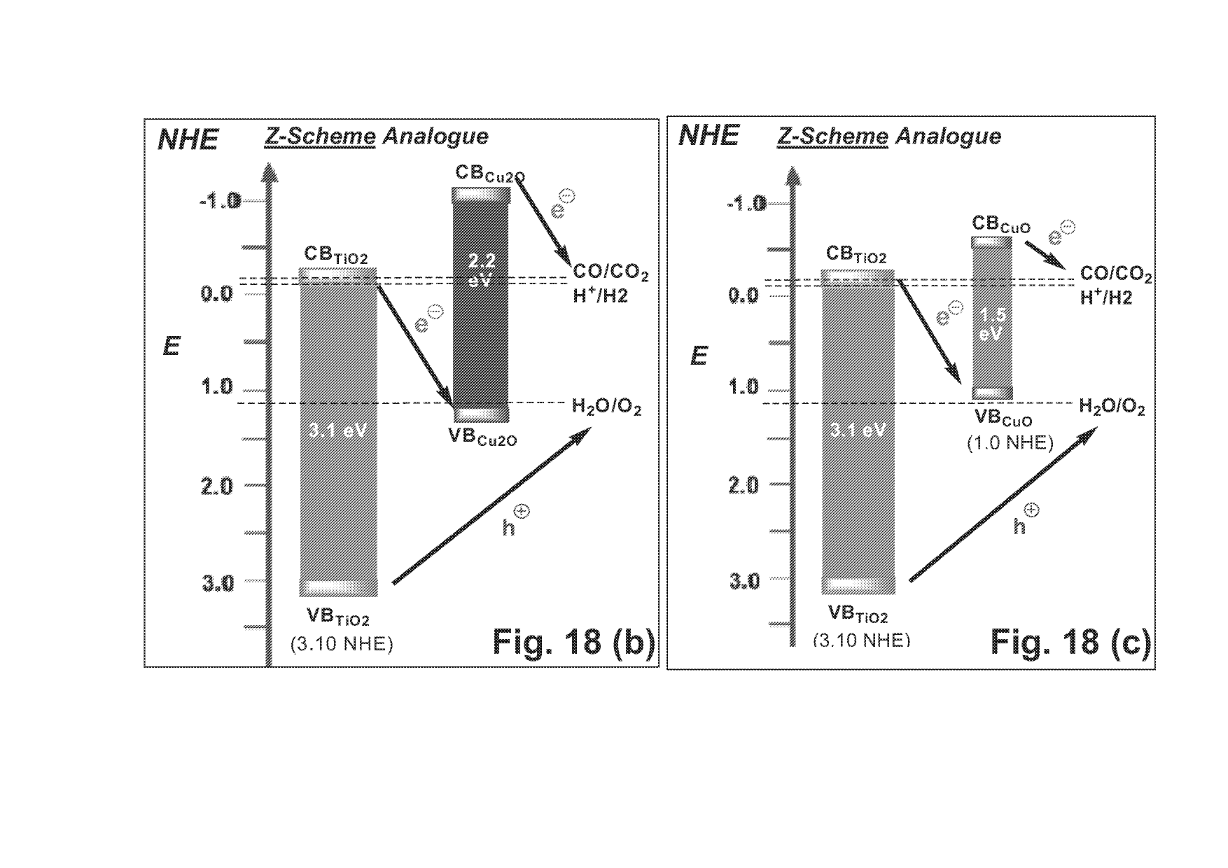

FIGS. 18A-18C show the pairing CuO/TiO₂, in both simple and Z-scheme electronic coupling. In this example, the CB of TiO₂ is around −0.15 to −0.35 eV (NHE), which is lower than the CB of CuO, at around −0.7 eV (NHE). Also, the VB of TiO₂ around 3.10 eV (NHE) is lower than the VB of CuO, at around 1.2 eV (NHE). The Eg for CuO is about 1.5 eV.

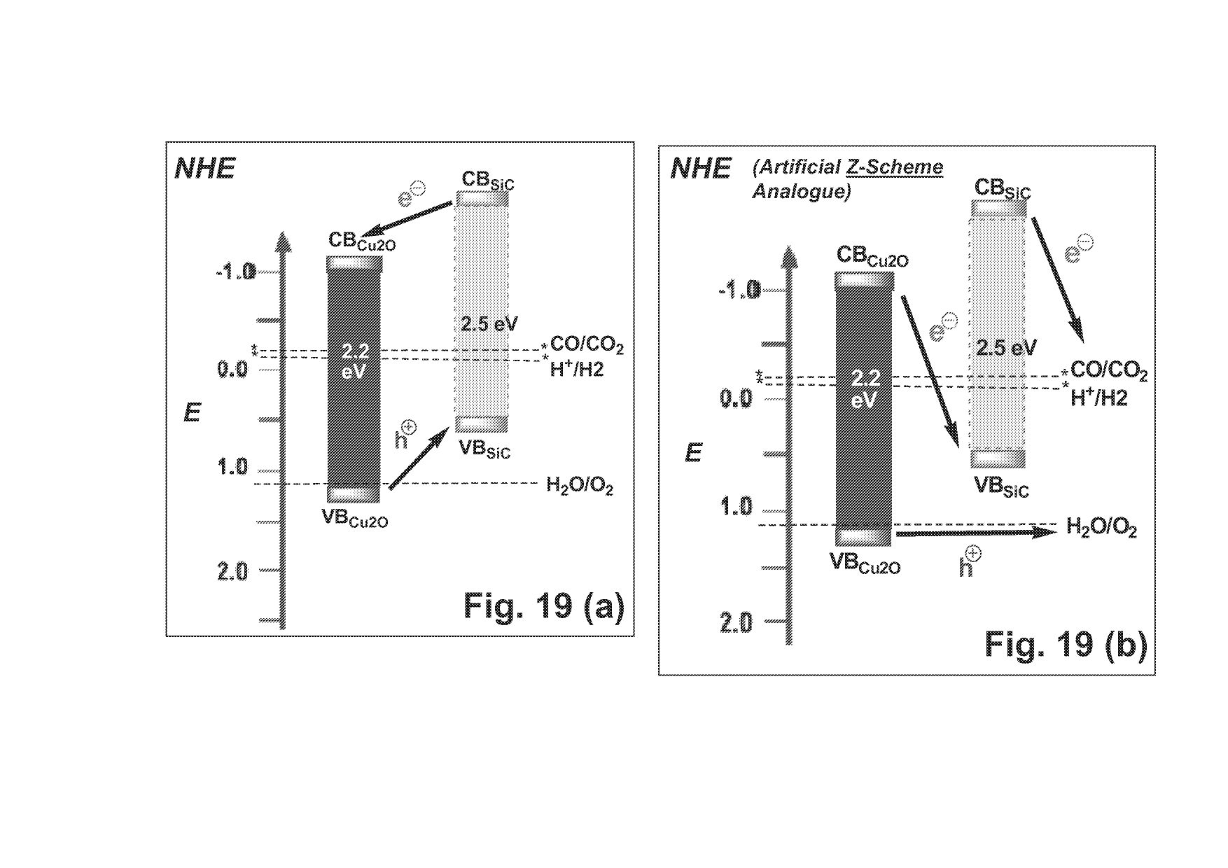

FIGS. 19A and 19B show the pairing SiC/Cu₂O, in both simple and Z-scheme electronic coupling. In this example, the CB of Cu₂O is around −0.9 to 1.1 eV (NHE), which is lower than the CB of SiC at around −2.0 eV (NHE). Also the VB of Cu₂O is around 1.2 to 1.4 eV (NHE), which is lower than the VB of SiC which is around 0.6 eV (NHE). In this case, the electronic Eg is about 2.2 eV for Cu₂O and about 2.5 eV for SiC.

In order to photoreact with light in the visible wavelength range (e.g., sunlight), lower Eg values are preferred. For example, constituents such as CuO (Eg=about 1.5 eV), Cu₂O (Eg=about 2.2 eV), SiC (Eg=about 2.5 eV) and Fe₂O₃ (Eg=about 2.8 eV) may be preferred as they are better able to absorb sunlight energy in the visible range of light (about 400 to 800 nm).

Selected relative VB and CB energies and Eg energies of adjacent nanoparticles of different photoactive constituents within a single layer or between nanoparticles of adjacent layers of different photoactive constituents enable efficient electronic coupling between photoactive constituents, and help to improve vectorial charge transport and charge carrier separation processes. These effects may be influenced by factors such as nanoparticle layer thickness, particle size, surface area, surface functionality, porosity, crystallinity and/or quantum size effects, among various others.

Pairing of two high band gap materials typically results in light absorption that is weaker in the visible wavelengths of light, but may provide good absorption for light outside the visible spectrum (e.g., in the UV range, which is considered to be above 400 nm). Pairing of two low band gap materials typically results in light absorption that is stronger in the visible spectrum (considered to be between 400 nm and 800 nm) and therefore would be more applicable to photoreactions using sunlight and/or CSP. Pairings of materials with different band gap values can be selected in order to obtain a desired range of absorption wavelengths. Mixing different pairings within the same photoactive material or combining two or more different photoactive materials into an assembly, as described below, can widen the range of absorption wavelengths.

In general, CB and VB levels should be paired to have one higher and one lower to allow charge carrier (i.e., electron and hole) separation pathways, which locate the generated charge carriers on separate nanoparticles. This would minimize recombination and would favor the described redox processes. CB and VB values, as well as pairings of constituents may be generally known in the literature¹³.

It should be noted that CB and VB values found in the literature are generally measured for bulk semiconductor materials. These values may be slightly different when measured for nanoparticle or thin film forms of these materials. However, selection of the materials and pairings can still be carried out based on the measurements of the bulk materials. In practice, the CB, VB and Eg energies may be typically determined by XPS or UPS measurements on the nanoparticles.

### Selection of Constituent Composition

Selection of the constituent photoactive nanoparticles begins with selecting the elemental composition of the nanoparticles. Selection can be made from the range of single, binary, ternary, quaternary or multi-metallic metal oxides, metal sulfides, metal silicides, metal borides, metal nitrides, metal phosphates, metal pnictides and metal carbides among others. The selection is largely based on the CB, VB and Eg values of the materials, as described above.

### Selection of Constituent Nanoparticle Size and Shape

The size of the constituent nanoparticles is also important. Typically, the size of the photoactive constituent nanoparticle is in the range of about 3-50 nm, and is chosen to be smaller than the thickness of the layer in which it is incorporated, in order to maintain a relatively flat surface at the interface between layers or with the air or substrate. The desired size can also depend on the specific compound. For example, it has been found that TiO₂ nanoparticles have better performance at particles sizes of about 12-15 nm diameter while CuO or Fe₂O₃ nanoparticles have better performance at particle sizes of about 5-8 nm diameter.

The optimum particle size for nanoparticles of each photoactive constituent can be determined experimentally⁸. Generally, it has been found that very small particles (e.g., 2-6 nm in diameter) exhibit lower photoactivity, perhaps due to an increase of surface defects which increase possible charge carrier recombination pathways. Larger particles (e.g., 10-15 nm in diameter) may have a higher degree of crystallinity and exhibit less surface defects.

It should be noted that while the nanoparticle size can be controlled with known synthesis methods, other treatments during manufacture of a nanoparticle layer or multi-layer, such as calcination, sintering and reduction processes, can affect the final size and shape.

The size of the constituent nanoparticles plays a role in the trade-off between high and low porosity, discussed above. It has been found that reaction rates showed dependence on the particle sizes in the layer. For example, comparing TiO₂ layers with large particles (≈20-25 nm), smaller particles (≈12-15 nm) and very small particles (≈4-6 nm) the preparation with particle sizes ≈12-15 nm showed the best photo activity. This was likely due to the trade-off between porosity and surface area. In these tests, the TiO₂ layer was paired with a Fe₂O₃ layer that was kept at a constant porosity with particle sizes of about 4-7 nm.

The sizes of the nanoparticles may be selected depending on the specific photoactive constituent. Different photoactive constituents may exhibit better photaoctivity for certain different ranges of nanoparticle sizes, which may be due to the differences in exciton diffusion length and surface defect density for the different photoactive constituents. The nanoparticles used in examples disclosed herein typically have diameters in the range of about 1 nm to about 1000 nm, more specifically about 1 nm to about 250 nm, more specifically about 1 nm to about 50 nm, in particular about 3 nm to about 25 nm. It should be understood that throughout this disclosure, although diameter is used to describe the size of the nanoparticles, the nanoparticles may not be spherical and may have any geometry as described below.

The shape of the nanoparticle can be a well-defined morphology with well-defined crystal facets or random in nature, or a mixture of both. For example, the nanoparticle may have a spherical, cubic, polyhedral, rod, wire, sheet or any other well-defined geometry. The shape of the nanoparticle is typically controlled during manufacture⁹. Typically, a higher degree of crystallinity, with a bigger grain size, is desirable as this may result in less surface defects on the nanoparticle and hence less chance of electron-hole recombinations.

The nanoparticle size distribution (PSD) is usually measured as a histogram of the population of a particular size versus the respective size, and is typically determined by electron microscopy or dynamic light scattering (DLS) studies. PSD is typically controlled during manufacture. Generally, the more equal and/or similar the particles are in their sizes, the lower the PSD value and the better the dispersion quality. PSD is mostly controlled through the synthesis process⁹, especially by the solvent and the reactants, surface charge and zeta potential.

Below is a table providing examples of different metal oxide nanoparticles made from metal powders, and their particles sizes as determined using scanning transmission electron microscopy (STEM), high resolution transmission electron microscopy (HRTEM) and powder X-ray diffraction (PXRD) with Rietveld refinement^{18,26}.

| Metal | Metal Oxide | STEM Size | Size range | |
|---|---|---|---|---|
| precursor | Composition^{[a]} | (nm)^{[b]} | (nm)^{[b]} | PXRD sizes^{[c]} |
| Mo | MoO₃ | 3.6 ± 0.5 | 2.5-4.1 | 4-5 (S1) |
| W | WO₃ | 3.8 ± 0.3 | 2.0-4.7 | 4-4.5 (S2) |
| Ni | NiO | 3.1 ± 0.4 | 2.2-3.7 | amorph. |
| Co | Co₃O₄ | 6.4 ± 2.7 | 4.5-8.3 | amorph. |
| Fe | Fe₂O₃ | 3.4 ± 0.5 | 2.7-4.5 | 3-3.5 (S4) |
| Zn | ZnO₂ (ZnO) | 3.9 ± 0.4 | 3.1-5.2 | 3-4.0 (S5) |
| Mg | MgO₂ (MgO) | 4.3 ± 0.9 | 3.2-5.7 | 4.5-5 (S6) |
| Mg + | MgCo₂O₄ | 21.4 ± 5.2 | 12-27 | 22 ± 4 (S7) |
| Co | | | | |
| Mg + | MgZn₂O₄ | 3.5 ± 0.4 | 2.8-4.6 | amorph. |
| Zn | | | | |
| Fe + | Fe_{0.3}Co_{0.7}MoO₄ | 3.1 ± 0.5 | 2.3-4.3 | 2.8-3.2 (S8) |
| Co + Mo | | | | |

Notes for the table above:
^{[a]}stable aqueous acidic H₂O₂ dispersion;
^{[b]}average particle size ranges as determined using HRTEM and Cryo-STEM measurements; and
^{[c]}particle sizes as determined using PXRD Rietveld refinement.

The following table provides some example metal oxide particle sizes observed from STEM and XRPD measurements, as well as Brunauer Emmett Teller (BET) surface area measurements. An alcoholic solvent was used in the synthesis of these nanoparticles.

| | Solvent or | | | |
|---|---|---|---|---|
| Metal Oxide | Solvent | Size (nm) | Size (nm) | BET |
| Nanoparticle | mixture | (STEM)^{[a]} | (XRPD)^{[b]} | (m²/g)^{[c]} |
| ZnO | Methanol | 3-5 | 3.9 ± 0.4 | 150.709 |
| ZnO | Ethanol | 6-12 | 10 ± 1.7 | 98.368 |
| ZnO | iso-Propanol | 17-45 | 42 ± 8.6 | 29.598 |
| (Fe₂O₃) | Methanol/ | — | — | — |
| | H₂O | | | |
| α-Fe₂O₃ | Ethanol/ | 4-7 | 4.6 ± 0.4^{[d]} | 242.224 |
| | H₂O | | 7.1 ± 1.2^{[e]} | |
| α-Fe₂O₃ | iso-Propanol/ | 10-22 | 17.6 ± 3 | 192.343 |
| | H₂O | | | |
| Fe₂O₃ | tert-Butanol/ | 12-25 | 17.9 ± 6^{[d]} | 115.712 |
| | H₂O | | 19.3 ± 9^{[f]} | |
| Fe₂O₃ | n-Propanol/ | 15-47 | 37.4 ± 9^{[d]} | 56.362 |
| | H₂O | | 19.3 ± 6^{[f]} | |

Notes for the table above:
^{[a]}STEM images were obtained using a Hitachi HD-2000 in the Z-contrast mode at an accelerating voltage of 200 kV and an emission current of 30-50 μA;
^{[b]}The crystal phase and particle size was analyzed by X-ray diffraction (XRD). The Rietveld refinement was carried out with Bruker AXS general profile fitting software Topas ™;
^{[c]}Physisorption measurement of 40 points adsorption/desorption isotherms, multi point (5 points) BET method was used to determine the surface area (g/m²);
^{[d]}Hematite phase;
^{[e]}Goethite phase; and
^{[f]}Maghematite phase.

Generally, particle size can be determined by XPRD from Rietfeld refinement calculation or from STEM, transmission electron microscopy (TEM) and/or HRTEM measurements.

The PSD (also referred as particle distribution (PD)) of various examples are provided below:

For MoO₃ in acidic H₂O₂/H₂O—Size (nm) is 3.6±0.5, PSD or PD is 0.14;

For NiO in acidic H₂O₂/H₂O—Size (nm) is 3.1±0.4, PSD or PD is 0.13;

For Fe₂O₃ in acidic H₂O₂/H₂O—Size (nm) is 3.4±0.5, PSD or PD is 0.15;

For MgZn₂O₄ in acidic H₂O₂/H₂O—Size (inn) is 3.5±0.4, PSD or PD is 0.11;

For ZnO in Methanol—Size (nm) is 3.9±0.4, PSD or PD is 0.1;

For ZnO in Ethanol—Size (nm) is 10±1.7, PSD or PD is 0.17;

For ZnO in Ethanol—Size (nm) is 42±8.6, PSD or PD is 0.20;

For α-Fe₂O₃ in iso-Propanol/H₂O—Size (nm) is 17.6±3, PSD or PD is 0.17

The PSD or PD values listed above were obtained by division of the standard deviation (±X) through the average number A e.g. (ZnO in Ethanol—Size (nm) is 10±1.7, PSD or PD is 0.17, where A is 10 and X is 1.7, therefore the PSD or PD number results in 0.17).

PSD values typically range between about 0.10 and 0.50. A good PSD value would be considered to fall in the range between about 0.10 and 0.35. In the examples discussed herein, most of the colloidal dispersions exhibit PSD values ranging from 0.12 to 0.44.

Optical transparency of the nanoparticle layer is important for good light penetration into the layer with minimal light scattering loss effects. High optical transparency is obtained when the nanoparticle constituents are smaller than the wavelength of light, since this results in less light scattering off the nanoparticles. The size of the nanoparticle also affects the values of valence band and conduction band energies, as well as the electronic band gap. It has been found in the examples described herein that smaller particle sizes result in larger Eg values, and higher VB and CB values, compared to the values measured from bulk reference materials, due to quantum size effects. However, as noted above, selection of constituents can still be carried out based on those values measured from bulk materials.

The surface area of the nanoparticles is another characteristic to be controlled. Typically, smaller nanoparticles have larger surface to volume (SN) ratio. This ratio can be measured by gas adsorption isotherms¹⁰. The SN ratio plays an important role in nanoparticle surface chemical reactions. The larger this ratio, the higher the number of surface active sites accessible to react with reactants adsorbed on the nanoparticle surface. Furthermore, SN ratios can be in general estimated from plots of percentage of surface-atoms of a nanoparticle as a function of the size/diameter of the nanoparticle. This is illustrated in the table below:

| Diameter | S (Surface) | V(Volume) |
|---|---|---|
| 1 nm | 13 | 1 |
| 2 nm | 9 | 1 |
| 5 nm | 1 | 1 |
| 10 nm | 3 | 7 |
| 20 nm | 1 | 4 |
| 60 nm | 1 | 9 |
| 100 nm | 1 | 20 |

For example, for nanoparticles having diameters in the range of about 1 nm to 100 nm, SN ratios will be in the range of about 13/1 (for 1 nm) up to 1/20 (for 100 nm).

It is usually desirable to have a higher S/V ratio. Typically, S/V values range between about 1 and 7. A good S/V value may be considered to lie in the range of about 5 to 7. The S/V ratio may be controlled through control of the particle sizes and porosity of the nanoparticle layer.

### Selection of Constituent Degree of Crystallinity

The degree of crystallinity of the constituent nanoparticles is another characteristic that can be controlled. Crystallinity can range from 100% amorphous (i.e., a completely random arrangement of constituent atoms) to 100% crystalline (i.e., a completely periodic arrangement of constituent atoms in a 1D, 2D or 3D lattice or crystal structure) and arrangements in between (e.g., semi-crystalline structures). This characteristic is typically difficult to quantify at the nanoscale and is usually done by high resolution electron microscopy (HRTEM), selected area electron diffraction (SAED) and powder X-ray diffraction (PXRD).

A good degree of crystallinity is about 95-100%, as determined from the measured diffraction pattern. Higher crystallinity, which is typically exhibited by larger particles, may play a role in better charge separation properties and higher photoactivity, perhaps by reducing surface defects thereby reducing the chances of electron-hole recombination. The degree of crystallinity is typically controlled during manufacture, in particular especially calcination conditions, since it has been found that calcination at higher temperatures generally result in to higher crystallinity. All nanoparticles of the same constituent should exhibit the same crystal structure and have similar degrees of crystallinity. Methods for controlling crystallinity and measuring crystallinity are generally known¹¹.

### Selection of Constituent Surface Charge

The surface charge of the constituent nanoparticle plays a role in manufacturing a film containing the nanoparticle. The surface charge is typically quantified by measuring the zeta potential. The surface charge on a nanoparticle can be positive, negative or zero. The surface charge is also controlled by pH and ionic strength of solvent in which the nanoparticle is dispersed. The isoelectric point is defined as the point of zero surface charge. Methods for controlling surface charge and its effects are generally known¹².

In the examples disclosed herein, the surface charge is generally controlled by the amount of protonated or de-protonated surfaces species. For example, a Fe₂O₃/EtOH dispersion at pH=2.26 resulted in a positive zeta-potential ζ of 20.1±1.1 mV, and a ZnO/EtOH dispersion at pH=7.16 resulted in a positive zeta potential ζ of 31.5±0.4 mV¹⁸.

The surface charge affects the colloidal forces between nanoparticles in a colloidal suspension since it determines the repulsive electrical double layer (EDL) and attractive Van der Waals (VDW) forces between nanoparticles suspended in the solvent. The balance of EDL and VDW forces controls the colloidal stability of the nanoparticles in the suspension.

Colloidal stability means that the nanoparticles do not agglomerate and do not flocculate or precipitate from the solvent. The quality of a nanoparticle film depends on the colloidal stability of the colloidal dispersion and hence the colloidal surface charge. During manufacturing, an optically transparent nanoparticle layer of controlled porosity and thickness is obtained by evaporation induced self assembly (EISA) through spin-coating if the nanoparticle dispersion in the chosen solvent is colloidally stable and does not flocculate during the film forming process.

Porosity of a manufactured nanoparticle layer, for example as high as 30-50% or 10-90% by volume, depends on the void spaces that form as the nanoparticles try to pack as efficiently as possible in the self-assembly process, which is driven by the balance of EDL and VDW forces between the nanoparticles. As explained above, a controlled degree of porosity is desirable to facilitate a balance between gas permeability and availability of reaction sites.

### Selection of Constituent Pairings

Photoreactions occur between pairings of two different photoactive constituents. The selection of these pairings is based on several characteristics.

The physical size, VB and CB energies, electronic band gap energy and composition of the photoactive constituent nanoparticles at least partly determine the optical transparency, surface area, porosity, gas diffusion and/or permeability behaviors of the photoactive material. These characteristics of the photoactive constituents also affect photoactivity and selectivity towards the generation of fuels, in particular methane and methanol (which may be produced in response to different wavelength ranges of incident light).

By “selectivity” towards generation of fuels, it is meant that the reaction preferentially produces a certain product, in this disclosure typically CH₄ or CH₃OH. This selectivity is based on properties such as the specific photoactive constituents as well the specific reaction conditions. For example, by using preferentially specific constituents such as CuO, Cu₂O or Cu⁰ metal in the photoactive material, the material may exhibit higher selectivity towards generation of CH₃OH.

Examples of selectivity of some photoactive constituents are shown in the table below:

| Constituents | Main photoreaction products |
|---|---|
| Cu/Fe* co-doped TiO₂ | Methane (CH₄) |
| Pt/TiO₂ or Ru/RuO₂/TiO₂ | Methane (CH₄) |
| Cu/ZnO/SiO₂ | Methanol (CH₃OH) |
| NiO/InTaO₄ | Methanol (CH₃OH) |
| Monoclinic BiVO₄ | Ethanol (C₂H₅OH) |

*Cu(0.25 wt %)/Fe(0.25 wt %)

The constituent pairing should also be selected such that the total light absorption is over as broad a wavelength range as possible. This photoelectric coupling is generally described in the literature¹³. For example, ZnO/TiO₂ may be considered a poor pairing since both semiconductors absorb mostly in the UV-part of the sunlight spectrum. A better pairing would be Fe₂O₃/TiO₂ where at least one constituent, specifically Fe₂O₃, possesses a stronger absorption in the visible range (400 nm to 800 nm). An even better example would be Fe₂O₃/CuO or Fe₂O₃/Cu₂O because both constituents absorb a broad wavelength of light, including the visible range. Another good combination would be SiC/CuO where SiC absorbs in the near infrared range and CuO absorbs in the visible range, thereby combining to provide light absorption in the near infrared and visible wavelength ranges.

Where the multi-layered photoactive material is arranged as a photonic crystal, the constituents may be selected to have large refractive index contrast (RIC) values, in order to achieve strong slow photon effects, as will be discussed below. A large RIC may be considered to be in the range of about 0.5 to 0.75 or 0.5 to 1.0. RI values for different bulk materials are generally known and can be found in various references and databases¹⁴. In general, RI is affected by the choice of constituent and degree of porosity and/or thickness of the resulting nanoparticle layer, examples of which will be described below.

Typically, the RIC between the layers of a multi-layered photoactive material is a function of the characteristics of the selected photoactive constituents and/or the porosity of the individual layers.

Electronic coupling between more photosensitive (i.e., narrower electronic band gap) and less photosensitive (i.e., wider electronic band gap) constituents may also have beneficial effects on the photoactive performance of the photoactive materials. Photosensitivity of a material may be determined by measuring the material's absorption of different wavelength ranges of light, particularly in the visible spectrum (i.e., about 400 to 700 nm). A less photosensitive material is considered to have absorption below 400 nm (e.g. ZnO or TiO₂ nanoparticles), while a more photosensitive material is considered to have absorption within the visible spectrum (e.g., CuO nanoparticles, which have absorption from about 700 to 350 nm or Fe₂O₃ nanoparticles, which have absorption from about 550 to 350 nm).

These pairings may be present as a mixture of the two constituents within a single-layer photoactive material; or may be present as separate layers of each constituent in a multi-layer photoactive material.

### Example Photoactive Constituents

Examples of photoactive constituents and their pairings that are suitable for a photoactive material are now described. These pairings are selected based on known electronic coupling between the photoactive constituents, as discussed above.

Example pairings include: TiO₂/WO₃, TiO₂/ZnO, TiO₂/CdSe, TiO₂/CuO, TiO₂/NiO, TiO₂/Fe₂O₃, WO₃/Fe₂O₃.

Examples of coupling between more photosensitive and less photosensitive constituents can be found in the following layer pairs:

TiO₂/SnO₂, TiO₂/ATO≡SnO₂:Sb, NiO/ATO≡SnO₂:Sb TiO₂/SiO₂, TiO₂/Al₂O₃ or TiO₂/ZrO₂.

To help improve the absorption of photons for photoactive reactions, a combination of optical absorption and electronic band properties may be selected. For instance, by combining relatively high electronic band gap metal oxide nanoparticles (e.g. TiO₂, ZnO, SnO₂, ATO≡SnO₂:Sb or mixed composition thereof) with relatively low electronic band gap metal oxide nanoparticles (e.g., Fe₂O₃, Co₂O₃, CuO, Cu₂O, RuO₂ or mixed composition thereof), the optical absorption properties of the photoactive material can be selected to occur at the energy of the lower electronic band gap constituent due to the convolution of the optical absorption properties of each layer, as discussed above.

Other examples of photoactive constituents and pairings are described in detail below. These photoactive constituent pairs can be used in the single-layer photoactive material and/or the multi-layered photoactive material, as will be discussed below.

### Example 1

### CuO/TiO₂ or Cu₂O/TiO₂ Pairs

Through electronic band gap engineering of the energy levels of nanoparticle constituents in a photoactive material, as described above, vectorial charge transport and charge carrier separation between the different photoactive constituents may be selected to favor a hole-rich layer and an electron-rich layer.

An example are the CuO/TiO₂ and Cu₂O/TiO₂ pairs, which may be arranged as alternating layers of CuO/TiO₂ or Cu₂O/TiO₂ or as mixed CuO/Cu₂O—TiO₂ layers, in a multi-layered photoactive material. These constituents may also be mixed together in a single-layer photoactive material. These constituent pairs may be arranged to achieve an optimal band-gap alignment.

In this example, the action of light may be described as follows:

TiO₂+CuO and/or Cu₂O+hν→CuO and/or Cu₂Oe_{CB}⁻+TiO₂h_{VB}⁺

In this example, the resulting CuO and Cu₂O electron-rich layers may participate in CO₂ reduction while the resulting TiO₂ hole-rich layers may concurrently enable H₂O oxidation. Reactions of this type may occur within or between layers of adjacent electronically-coupled nanoparticle layers in a multi-layered photoactive material; or within a single-layer of mixed nanoparticles in a single-layer photoactive material.

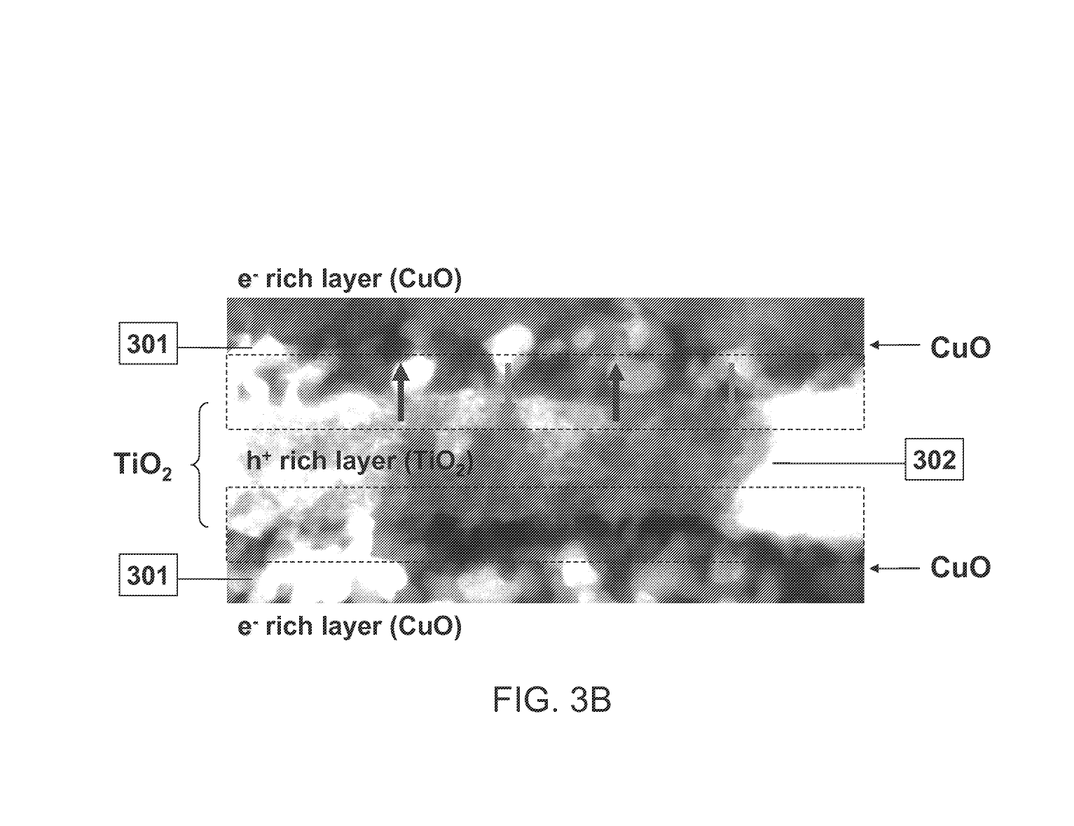

FIGS. 3A-3B illustrate electronic band gap engineering of the example CuO/TiO₂ pairing. It should be understood that although the redox reaction is illustrated here and in later examples with respect to a multi-layered photoactive material made of layers of different photoactive constituents, such a reaction can also take place within a single-layer photoactive material containing a mixture of at least two different photoactive constituents.

FIG. 3A-B illustrates the formation of charge carriers and the redox of CO₂ and H₂O that is enabled in a photoactive material having nanoparticle CuO/TiO₂ layers. In the example shown, the CuO layers 301 alternate with TiO₂ layers 302. The CuO layers 301 undergo activation/reduction of CO₂ while the TiO₂ layers 302 undergo oxidation of water, resulting in the reduction and activation of CO₂ or the generation of H₂.

The photoreactions carried out in the photoactive material include simultaneous oxidation and reduction, such as exemplified by the reactions CO₂+H₂O and CO₂+H₂, in particular the concurrent oxidative splitting of water and reduction of CO₂ as illustrated below:

CO₂+4H₂→CH₄ (g)+2H₂O and

CO₂+2H₂O→CH₃OH(1)+ 3/2O₂

The product water may be re-used and/or recycled or split in situ in additional reactions with the hole-rich and electron-rich species, as shown in the example below.

The following equations illustrate reactions that may take place within a photoactive material:

(electron-rich reaction) CO₂+CuO and/or Cu₂Oe_{CB}⁻→(CO₂⁻)*

2H⁺+CuO and/or Cu₂Oe_{CB}⁻→H₂ or (2H)

(hole-rich reaction) H₂O+TiO₂h_{VB}⁺→—OH+H⁺

OH+H⁺+TiO₂h_{VB}⁺→½O₂ (g)+2H⁺

(CO₂⁻)*+2H+TiO₂h_{VB}⁺→CO+H₂O

(CO)*+6H+TiO₂h_{VB}⁺→CH₄+H₂O

(CO₂⁻)*+8H+TiO₂h_{VB}⁺→CH₄+2H₂O

where * indicates an activated state of a compound. The redox processes for the photon-driven generation of adjacent electron- and hole-rich species are designed, through electronic band energy and electronic band gap engineering, as described above, to enable the concurrent reduction and oxidation of CO₂ and H₂O respectively. These processes may occur in a multi-layered photoactive material as well as in a single-layer photoactive material.

### Example 2

### TiO₂/WO₃ Pairs

In this example, the photoactive constituents include photoactive TiO₂ nanoparticles and photoactive WO₃ nanoparticles. In this example, similar to example 1 above, the action of light is described by the reduction of CO₂ and oxidation of H₂O within a photoactive material, whether single-layer or multi-layered, according to the following reaction equations:

TiO₂+WO₃+hν→WO₃e_{CB}⁻+TiO₂h_{VB}⁺

(electron-rich reaction) CO₂+WO₃e_{CB}⁻→(CO₂⁻)*

2H⁺+WO₃e_{CB}⁻→H₂ or (2H)

(hole-rich reaction) H₂O+TiO₂h_{VB}⁺→—OH+H

OH+H⁺+TiO₂h_{VB}⁺→½O₂ (g)+2H⁺

(CO₂⁻)*+2H+TiO₂h_{VB}⁺→CO+H₂O

(CO)*+6H+TiO₂h_{VB}⁺→CH₄+H₂O

(CO₂⁻)*+8H+TiO₂h_{VB}⁺→CH₄+2H₂O

where * indicates an activated state of a compound. The redox processes for the photon-driven generation of adjacent electron- and hole-rich species are designed, through electronic band energy and electronic band gap engineering, as described above, to enable the concurrent reduction and oxidation of CO₂ and H₂O respectively. These processes may occur in a multi-layered photoactive material as well as in a single-layer photoactive material.

### Example 3

### α-Fe₂O₃/TiO₂ Pairs

In this example, the photoactive constituents include photoactive TiO₂ nanoparticles and photoactive α-Fe₂O₃ (hematite) nanoparticles. In this example, similar to example 1 above, the action of light is described by the reduction of CO₂ and oxidation of H₂O within a photoactive material, whether single-layer or multi-layered, according to the following reaction equations:

TiO₂+α-Fe₂O₃+hν→α-Fe₂O₃e_{CB}⁻+TiO₂h_{VB}⁺

(electron-rich reaction) CO₂+α-Fe₂O₃e_{CB}⁻→(CO₂⁻)*

2H⁺+α-Fe₂O₃eCB⁻→H₂ or (2H)

(hole-rich reaction) H₂O+TiO₂h_{VB}⁺→—OH+H

OH+H⁺+TiO₂h_{VB}⁺→½O₂ (g)+2H⁺

(CO₂⁻)*+2H+TiO₂h_{VB}⁺→CO+H₂O

(CO)*+6H+TiO₂h_{VB}⁺→CH₄+H₂O

(CO₂⁻)*+8H+TiO₂h_{VB}⁺→CH₄+2H₂O

where * indicates an activated state of a compound. The redox processes for the photon-driven generation of adjacent electron- and hole-rich species are designed, through electronic band energy and electronic band gap engineering, as described above, to enable the concurrent reduction and oxidation of CO₂ and H₂O respectively. These processes may occur in a multi-layered photoactive material as well as in a single-layer photoactive material.

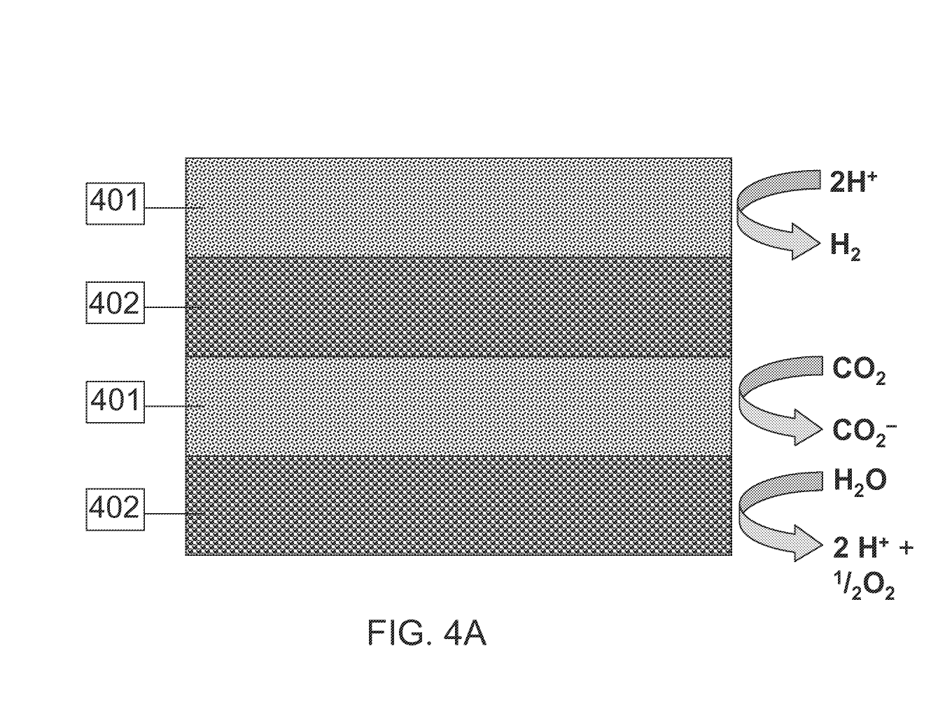

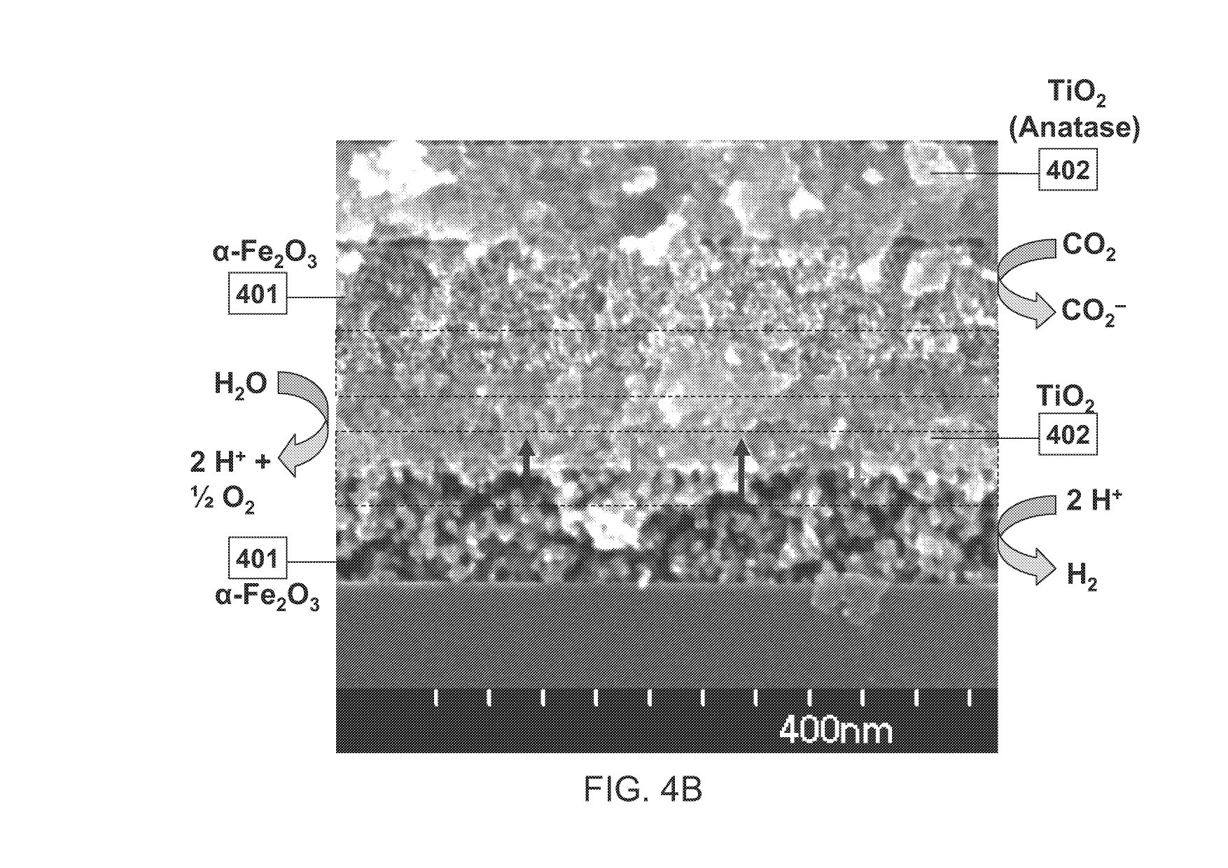

FIG. 4A-B illustrates the formation of charge carriers and the redox of CO₂ and H₂O that is enabled in a photoactive material having nanoparticle Fe₂O₃/TiO₂ layers. In the example shown, the TiO₂ layers 402 alternate with Fe₂O₃ layers 401. The Fe₂O₃ layers 401 undergo activation/reduction of CO₂ while the TiO₂ layers 402 undergo oxidation of H₂O, resulting in the reduction and activation of CO₂ or the generation of H₂.

### Example 4

### Cu(Cu₂O)/α-Fe₂O₃ Pairs

In this example, the photoactive constituents include photoactive Cu(CuO) and Fe₂O₃ nanoparticles. These constituents may be mixed together in a single-layer photoactive material, or as separate layers in a multi-layered photoactive material.

Similar to example 1 above, electronic coupling between different photoactive nanoparticles leads to an improved charge carrier production and separation of electron-hole pairs, and the copper nanoparticles enables improved photoactive activity.

Additionally, in this example, the copper nanoparticles may give rise to plasmonic resonance, which enhances the absorption of light and the photoactivity of a photoactive material incorporating Cu(Cu₂O)/α-Fe₂O₃. This will be described in greater detail below.

Interfaces between the electron-rich Cu and hole-rich Fe₂O₃ nanoparticles may also function as a Schottky barrier, which suppresses electron-hole recombination processes.

A Schottky barrier is defined as the interface, boundary or electronic interface between a metal and a semiconductor¹³. The Schottky barrier serves to suppress electron-hole recombination processes, as the electron gets trapped within the metal (e.g. Cu, Ag, Au or Pt) and the hole remains on the more acidic metal oxide (e.g. Fe₂O₃, TiO₂, WO₃, among others).

In this example, similar to example 1 above, the action of light is described by the reduction of CO₂ and oxidation of H₂O that may take place within a photoactive material, whether single-layer or multi-layered, according to the following reaction equations:

Cu(CuO)+Fe₂O₃+hν→Cu(CuO)e_{CB}⁻+Fe₂O₃h_{VB}⁺

(electron-rich reaction) CO₂+Cu(CuO)e_{CB}⁻→(CO₂⁻)*

2H⁺+Cu(CuO)e_{CB}⁻→H₂ or (2H)

(hole-rich reaction) H₂O+Fe₂O₃h_{VB}⁺→—OH+H

OH+H⁺+Fe₂O₃h_{VB}⁺→½O₂ (g)+2H⁺

(CO₂⁻)*+2H+Fe₂O₃h_{VB}⁺→CO+H₂O

(CO)*+6H+Fe₂O₃h_{VB}⁺→CH₄+H₂O

(CO₂⁻)*+6H+Fe₂O₃h_{VB}⁺→CH₃OH+H₂O

where * indicates an activated state of a compound. The redox processes for the photon-driven generation of adjacent electron- and hole-rich species are designed, through electronic band energy and electronic band gap engineering, as described above, to enable the concurrent reduction and oxidation of CO₂ and H₂O respectively. These processes may occur in a multi-layered photoactive material as well as in a single-layer photoactive material.

### Example 5

### Cu₂O/SiC Pairs

In this example, the photoactive constituents include photoactive Cu₂O nanoparticles and photoactive SiC nanoparticles. In this example, similar to example 1 above, the action of light is described by the reduction of CO₂ and oxidation of H₂O that may take place within a photoactive material, whether single-layer or multi-layered, according to the following reaction equations:

SiC+Cu₂O+hν→SiCe_{CB}⁺+Cu₂Oh_{VB}⁺

(electron-rich reaction) CO₂+SiCe_{CB}⁻→(CO₂⁻)*

2H⁺+SiC_{CB}⁻→H₂ or (2H)

(hole-rich reaction) H₂O+Cu₂Oh_{VB}⁺→—OH+H

OH+H⁺+Cu₂Oh_{VB}⁺→½O₂ (g)+2H⁺

(CO₂⁻)+2H+Cu₂Oh_{VB}⁺→CO+H₂O

(CO)*+6H+Cu₂Oh_{VB}⁺→CH₄+H₂O

(CO₂⁻)*+8H+Cu₂Oh_{VB}⁺→CH₄+2H₂O

where * indicates an activated state of a compound. The redox processes for the photon-driven generation of adjacent electron- and hole-rich species are designed, through electronic band energy and electronic band gap engineering, as described above, to enable the concurrent reduction and oxidation of CO₂ and H₂O respectively. These processes may occur in a multi-layered photoactive material as well as in a single-layer photoactive material.

### Other Examples

Other example photoactive constituents are described below. These are selectable from known earth-abundant, easy to synthesize, colloidally stable, inexpensive and/or non-toxic metal oxides. Such metal oxides include, for example, constituent pairs having the general stoichiometric formulation: M¹ₙO_{y}/M²ₙOₓ; M¹ₙO_{y}-M²ₙO_{y}/M³ₙO_{z}; M¹ₙO_{y}-M²ₙO_{y}/M³ₙO_{z}-M⁴ₙO_{z}; MⁿₙO_{y}/MⁿₙO_{z} (where M is a suitable metal and n, x, y, z are integers). The constituents may also include mixed compositions, solid-solution, combinations with other semiconductor materials, as well as non-stoichiometric compositions (e.g. MO wherein 0.1≦x≦1), and/or combinations thereof. It should be understood that in the present disclosure, the term non-stoichiometric is intended to include sub-stoichiometric compositions.

In particular, suitable photoactive constituent pairs include:

Fe₂O₃/TiO₂; Fe₂O₃/WO₃; ZnO/TiO₂; ZnO/WO₃; CuO/Fe₂O₃; CuO—ZnO/Fe₂O₃; CuO/TiO₂; CuO/WO₃; CuO—ZnO/TiO₂; CuO—ZnO/WO₃; CuO—Fe₂O₃/ZnO; CoO/TiO₂; Co₃O₄/WO₃; Co₃O₄—ZnO/TiO₂; Co₃O₄—Fe₂O₃/WO₃; CuO—Co₃O₄/Fe₂O₃; CeO₂/Fe₂O₃; CeO₂/TiO₂; CeO₂/WO₃; CeO₂—NiO/TiO₂; CoO—CeO₂/WO₃; ATO/Fe₂O₃; Fe₂O₃/NiO—Co₃O₄; Cu₂O-ATO/Fe₂O₃; NiO/Fe₂O₃; NiO/TiO₂; SiC/CuO; ITO/WO₃; Cu₂O/Fe₂O₃; Cu₂O/TiO₂; ATO-CuO/SiC; NiO—Fe₂O₃/Cu₂O; SiC/Cu₂O; SiC—Cu₂O/Fe₂O₃; TiO₂/WO₃; Fe₂O₃—CuO/NiO; Fe₂O₃—NiO/CuO; ZnFe₂O₄/TiO₂; MgCo₂O₄/WO₃; TiO₂/ATO; Fe₂O₃—CuO/ATO; BiVO₄/NiO; Bi₂WO₆/Cu₂O; ITO-Cu₂O/WO₃ and NiWO₄/Fe₂O₃—Cu₂O.

Further, the following species are known to be suitable for photoactive constituents¹³:

I) Simple Metal-Oxides e.g.: in all known modifications and polymorphs e.g. α-; β-; γ-; δ- as well as all possible non-stoichiometric compositions and/or combinations thereof MOₓ wherein 0.1≦x≦1.
Al₂O₃, AlOOH, and all known modifications and polymorphs e.g. α-; β-; γ-; δ-
FeO, FeO(OH), Fe(OH)₃, Fe₂O₃, Fe₃O₄ and all known modifications and polymorphs e.g. α-; β-;
TiO₂ (rutile, anatase & brookite-phase); SnO₂, Ti₂O₃ and all known modifications and polymorphs e.g. α-; β-; γ-; δ-
MgO, CaO, SrO, BaO, CoO and all known modifications and polymorphs e.g. α-; β-; γ-; δ-
CuO, Cu₂O, NiO, ZnO, BeO and all known modifications and polymorphs e.g. α-; β-; γ-; δ-
WO₃, MoO₃ and all known modifications and polymorphs e.g. α-; β-; γ-; δ-
SiO₂, B₂O₃, GeO₂, MnO₂ and all known modifications and polymorphs e.g. α-; β-; γ-; δ-
Ta₂O₅, Nb₂O₅, V₂O₅, Co₃O₄ and all known modifications and polymorphs e.g. α-; β-; γ-; δ-
Ga₂O₃, Cr₂O₃, Mn₂O₃, V₂O₃, Nb₂O₃ and all known modifications and polymorphs α-; β-; γ-; δ-
La₂O₃, Bi₂O₃, Sb₂O₅ and all known modifications and polymorphs e.g. α-; β-; γ-; δ-
SnO₂, ZrO₂, CeO₂, VO₂, ThO₂, TeO₂ and all known modifications and polymorphs α-; β-; γ-; δ-
Ag₂O, PdO, RuO₂, Au₂O, IrO₂, Re₂O₇ and all known modifications and polymorphs α-; β-; γ-; δ-
P₂O₅, P₄O₁₀ in all known modifications and polymorphs α-; β-; γ-; δ-
Transparent conductive metal oxides (TCOs), e.g. ITO≡In₂O₅:Sn (Indium Tin Oxide), ATO≡SnO₂:Sb (Antimony Tin Oxide), FTO≡SnO₂:F (Flourine Tin Oxide), ZTO≡SnO₂:Zn (Zinc Tin Oxide), IZO≡In₂O₅:Zn (Indium Zinc Oxide) as well as various mixtures thereof and with any other photoactive semiconductor materials, including solid solutions, core@shell e.g. MOs@TCO structures in all known and possible modifications, non-stoichiometric compositions and/or combinations thereof and/or different doping levels and polymorphs α-; β-; γ-; δ-.
Porous metallic films, resulting from reduction of metal oxide components, e.g. porous Au, Ag, Cu, p-Si, porous Si, crystalline Si, amorphous Si, porous Si nanowires¹⁵, as well as various mixtures thereof and with any other photoactive materials, including various alloys M1-M2 and core@shell e.g. M1@M2.
   II) Mixed Metal-Oxides e.g.: in all known modifications and polymorphs α-; β-; γ-; δ- as well as all possible non-stoichiometric compositions (e.g., M¹M²O_{X} wherein 0.1≦x≦1) and/or combinations thereof.
Rock-salt solid solutions e.g. (Mg₁₋ₓCaₓO) (wherein 0.1≦x≦1)
Ca₁₋ₓBiₓVₓMo₁₋ₓO₄ solid solutions (wherein 0.1≦x≦1)
Na₁₋ₓLaₓTa₁₋ₓCoₓO₃ solid solutions (wherein 0.1≦x≦1)
(AgNbO₃)₁₋ₓ(NaNbO₃)ₓ solid solutions (wherein 0.1≦x≦1)
Corundum solid solutions e.g. (FeCr)₂O₃
Spinels AB₂O₄ e.g. (MgAl₂O₄)
Ilmenites ABO₃ e.g. (FeTiO₃)
Perovskites ABO₃ e.g. (CaTiO₃)
Olivins A₂BO₄ e.g. (Mg₂SiO₄)
Granates A(II)₃B(III)₂Si₃O₁₂ e.g. (Fe₃Al₂Si₃O₁₂)
Gallium and Zinc nitrogen oxide (Ga₁₋ₓZnₓ)(N₁₋ₓOₓ) (wherein 0.1≦x≦1)
Ti-silicates (TiO₂ in SiO₂)
Aluminas and Silicated Aluminas (Si—Al₂O₃)
Polyoxymetallates in general (e.g., [EW₁₀O₃₆]ⁿ⁻¹² or [EMo₁₂O₄₂]ⁿ⁻¹²)
   III) Multicomponent Mixed Metal-Oxides (which may be photoactive for visible light irradiation): e.g. in all known modifications and polymorphs α-; β-; γ-; δ- as well as all possible non-stoichiometric compositions and/or combinations thereof e.g. MₐM_{b}M_{c}Oₓ wherein 0≦x≦1
BiVO₄, Vi₂WO₆, Bi₂MoO₆, NiWO₄, InVO₄, CaInO₄, InNbO₄, Pb₃Nb₄O₁₃, BaBiO₃, CaBi₂O₄, AgAlO2, Ag₂CrO₄, AgCrO₂, AgInW₂O₈, PbBi₂Nb₂O₉, Zn_{2.5}VMoO₈, In₁₂NiCr₂Ti₁₀O₄₂, In₁₋ₓNiₓTaO₄, InTaO₄, SrTiO3, La₂Ti₂O₇, LaTiO₅, Sr₃Ti₂O₇, BaTi₄O₉, PbTiO₃, or M₂Ti₆O₁₂ (M=Na, K, Rb), Fe_{0.3}CoO_{0.7}MoO₄, K₄Nb₆O₁₇, KCa₂Nb₃O₁₀, KNb₃O₈, KTiNbO₅, M₂BiNbO₇ (M=Ca, In, Ln), H₂SrTa₂O₇, NaTaO₃, LnTaO₄, M_{0.5}Nb_{0.5}O₃ (M=Ca, Sr, Ba), K₄Ce₂Nb₁₀O₃₀, PbBi₂Nb₂O₉, In₆NiTi₆O₂₂, In₃CrTi₂O₁₀, In₁₂NiCr₂Ti₁₀O₄₂, Nb₂Zr₂O₁₇₋ₓN₂, Nb₂Zr₆O₁₇, or generally:
M^{a}₁₋ₓM^{b}ₓO_{y} or Mⁿ₁₋ₓM^{m}ₓO_{y}; M^{a}₁₋ₓM^{b}ₓM^{c}O_{y}
M^{a}₁₋ₘM^{b}ₐM^{c}_{b}M^{d}_{c}MⁿₘO_{y}
   IV) Metal carbides in general, in all known modifications and polymorphs α-; β-; γ-; δ- as well as all possible non-stoichiometric compositions and/or combinations thereof (e.g. Ta₄C₃, Nb₄C₃, Mo₃C₂, Fe₃C, SiC);
   V) Metal nitrides in general, in all known modifications and polymorphs α-; β-; γ-; δ- as well as all possible non-stoichiometric compositions and/or combinations thereof (e.g. Ta₃N₅, TiN, Si₃N₄). This may include metal-(oxy)nitrides in general in all known modifications and polymorphs α-; β-; γ-; δ- as well as all possible non-stoichiometric compositions and/or combinations thereof (e.g. GaN, Ge₃N₄, GeN₄, TaON, Zr₂O₂N₂, Y₂Ta₂O₅N₂)
   VI) Metal borates and borides in general, in all known modifications and polymorphs α-; β-; γ-; δ- as well as all possible non-stoichiometric compositions and/or combinations thereof (e.g. Ni(BO₂)₂×H₂O, Co(BO₂)₂, YB₆, REAlB₁₄);
   VII) Chalcogenides in general, e.g. Metal sulfides in all known modifications and polymorphs α-; β-; γ-; δ- as well as all possible non-stoichiometric compositions and/or combinations thereof (e.g. Ag₂S, ZnS, MoS₂, WS₂, CdS, AgInS₂, FeS₂, ZnIn₂S₄);
   VIII) Metal chalcogenides in general, in all known modifications and polymorphs α-; β-; γ-; δ- as well as all possible non-stoichiometric compositions and/or combinations thereof (e.g., CdSe; ZnSe, CIGS (Copper indium gallium selenides);
   IX) Metal phosphate, -polyphosphates and phosphides in general, in all known modifications and polymorphs α-; β-; γ-; δ- as well as all possible non-stoichiometric compositions and/or combinations thereof (e.g. Ag₃(PO₄), Co₃(PO₄)₂, Cu₂(PO₄)OH, Ni₃(PO₄)₂, Zn₃(PO₄)₂, Zn₃P₂, TiP, InP, GaP)
   X) Metal arsenides in general, in all known modifications and polymorphs α-; β-; γ-; δ- as well as all possible non-stoichiometric compositions and/or combinations thereof (e.g. GaAs, InAs)
   Note that metal nitrides, metal phosphides and metal arsenides generally fall into the class of metal pnictides.
   XI) Metal silicides in general, in all known modifications and polymorphs α-; β-; γ-; δ- as well as all possible non-stoichiometric compositions and/or combinations thereof (e.g. NiSi, WSi₂, PtSi, TiSi₂)
   XII) Metal-oxy-sulfides and metal oxyhalides in general, in all known modifications and polymorphs α-; β-; γ-; δ- as well as all possible non-stoichiometric compositions and/or combinations thereof (e.g. Bi₄NbO₈Cl, AgClO₂)

For IV) to XII) above, also all known modifications e.g. α-; β-; γ-; δ-; ∈-; η-; θ-; as well as all possible non-stoichiometric compositions and/or combinations thereof, all known polymorphs and/or further mixed phases of the above, which can occur also as mixed oxy-hydroxyl species, among various others possible combinations.

XIII) Organic semiconductors, porous semiconductor polymers and carbon compounds (e.g., carbon, graphite, diamond, carbon nitride, g-C₃N₄ and all known modifications polymorphs α-; β-; γ-; δ-; ∈-; η-; θ-; as well all as possible non-stoichiometric compositions and/or combinations thereof etc.)
XIV) Up-converter nanocrystals in general in all known modifications and polymorphs α-; β-; γ-; δ- as well as all possible non-stoichiometric compositions and/or combinations thereof (e.g. NaYF₄, LaF₃, Y₂O₃, Gd₂O₃, Nd₂O₃, Er₂O₃, Sm₂O₃, Gd₂O₃ and their doped or codoped systems with e.g. Er³⁺ and/or Yb³⁺).

The metal oxides used may include the simple metal oxide and all their known modifications e.g. α-; β-; γ-; ∈-; η-; θ-; as well as all possible non-stoichiometric compositions and/or combinations thereof. The stoichiometric compositions may be generally denoted as MₙO_{y} (where n and y are integers), (e.g., TiO₂, WO₃, SnO₂, ITO≡In₂O₅:Sn (Indium Tin Oxide), ATO≡SnO₂:Sb (Antimony Tin Oxide), FTO≡SnO₂:F (Flourine Tin Oxide), ZTO≡SnO₂:Zn (Zinc Tin Oxide), IZO≡In₂O₅:Zn (Indium Zinc Oxide), ZnO or Fe₂O₃ as well as various possible mixtures thereof).

They may further include bimetallic mixed metal oxides (e.g., non-stoichiometric compositions M^{a}₁₋ₓM^{b}ₓO and stoichiometric compositions (M^{a}M^{b})ₙO_{y}, AB₂O₄, ABO₃), multi-metallic and multi-component metal oxide composites and compositions as core@shell structures M¹O@M²O (e.g. CuO@Fe₂O₃, Cu₂O@CuO, FeTiO₃CuO@Cu₂O, NiO@Cu₂O), as well as heterodimeric nanoparticle assemblies M¹O-M²O (e.g., NiO—CuO, CuO—Fe₂O₃, MnFe₂O₄—Cu₂O). Additionally, the different metal-oxide compounds and compositions may be doped and co-doped with or by the following metallic and non-metallic dopants and co-dopants in their different occurring oxidation states e.g. Mⁿ⁺, with n=1 to 8.
for example with the following non-metallic dopants “D”: B, Si, C, S, Se, P, As, F, N, I, that may be generally denoted, in non-stoichiometric compositions, as M_{1-y}D_{y}Oₓ wherein 0.1≦x and y≦1 and all possible non-stoichiometric compositions and/or combinations thereof.
for example with the following metallic dopants “D”: Be, Li, K, Mg, Ca, Sc, Y, Ti, Zr, Hf, V, Nb, Ta, Cr, Mo, W, Mn, (Tc), Re, Fe, Ru, Os, Co, Rh, Ir, Ni, Pd, Pt, Cu, Ag, Au, Zn, Cd, (Hg), Al, Ga, In, Tl, Ge, Sn, Pb, Sb, Bi, Te, Po, At, La, Ce, Pr, Nd, Eu, Gd, Tb, Dy, Ho, Er, Tm, Yb, Lu, Ac, Th, Pa, U, Pu; generally denoted, in non-stoichiometric compositions, as M_{1-y}D_{y}Oₓ wherein 0.1≦x and y≦1 and all possible non-stoichiometric compositions and/or combinations thereof.

The disclosed photoactive materials may also incorporate metal oxides (MₙO_{y}), bimetallic mixed metal-oxides (e.g., non-stoichiometric compositions M^{a}₁₋ₓM^{b}ₓO and stoichiometric compositions (M^{a}M^{b})ₙO_{y}, AB₂O₄, ABO₃), multi-metallic and multi-component metal oxide composites, as well as non-stoichiometric compositions and/or combinations thereof.

Other examples of photoactive constituents and their pairings that may be suitable for use in the disclosed photoactive material are shown in the tables below (--- indicates no pairing, xxx indicates the pairing did not exhibit a photonic stop band):

Pairings with Metal Oxides:

| | TiO₂ | ZnO | Fe₂O₃ | WO₃ |
|---|---|---|---|---|
| TiO₂ | — | TiO₂/ZnO | TiO₂/Fe₂O₃ | TiO₂/WO₃ |
| ZnO | ZnO/TiO₂ | — | XXX | ZnO/WO₃ |
| Fe₂O₃ | Fe₂O₃/TiO₂ | XXX | — | Fe₂O₃/WO₃ |
| WO₃ | WO₃/TiO₂ | WO₃/ZnO | WO₃/Fe₂O₃ | — |
| CuO | CuO/TiO₂ | XXX | XXX | CuO/WO₃ |
| NiO | NiO/TiO₂ | XXX | XXX | NiO/WO₃ |
| SnO₂ | SnO₂/TiO₂ | XXX | XXX | SnO₂/WO₃ |
| SiO₂ | SiO₂/TiO₂ | XXX | XXX | SiO₂/WO₃ |
| MgO/MgF₂ | MgO or | XXX | XXX | MgO or |
| | MgF₂/TiO₂ | | | MgF₂/WO₃ |
| Al₂O₃ | Al₂O₃/TiO₂ | XXX | XXX | Al₂O₃/WO₃ |
| ATO | ATO/TiO₂ | XXX | XXX | ATO/WO₃ |
| ITO | ITO/TiO₂ | ITO/ZnO | ITO/Fe₂O₃ | ITO/WO₃ |

Pairings with Catalytic Constituents:

| | CuO | NiO | SnO₂ |
|---|---|---|---|
| TiO₂ | TiO₂/CuO | TiO₂/NiO | TiO₂/SnO₂ |
| ZnO | XXX | XXX | XXX |
| Fe₂O₃ | XXX | XXX | XXX |
| WO₃ | WO₃/CuO | WO₃/NiO | WO₃/SnO₂ |
| CuO | — | XXX | XXX |
| NiO | XXX | — | XXX |
| SnO₂ | XXX | XXX | — |
| SiO₂ | XXX | XXX | XXX |
| MgO/MgF₂ | XXX | XXX | XXX |
| Al₂O₃ | XXX | XXX | XXX |
| ATO | XXX | XXX | XXX |
| ITO | ITO/CuO | ITO/NiO | ITO/SnO₂ |

Pairings with Low Refractive Metal Oxides:

| | SiO₂ | MgO/MgF₂ | Al₂O₃ |
|---|---|---|---|
| TiO₂ | TiO₂/SiO₂ | TiO₂/MgO or MgF₂ | TiO₂/Al₂O₃ |
| ZnO | XXX | XXX | XXX |
| Fe₂O₃ | XXX | XXX | XXX |
| WO₃ | WO₃/SiO₂ | WO₃/MgO or MgF₂ | WO₃/Al₂O₃ |
| CuO | XXX | XXX | XXX |
| NiO | XXX | XXX | XXX |
| SnO₂ | XXX | XXX | XXX |
| SiO₂ | — | XXX | XXX |
| MgO/MgF₂ | XXX | — | XXX |
| Al₂O₃ | XXX | XXX | — |
| ATO | XXX | XXX | XXX |
| ITO | ITO/SiO₂ | ITO/MgO or MgF₂ | ITO/Al₂O₃ |

Pairings with Conductive Metal Oxides:

| | ATO | ITO |
|---|---|---|
| TiO₂ | TiO₂/ATO | TiO₂/ITO |
| ZnO | XXX | ZnO/ITO |
| Fe₂O₃ | XXX | Fe₂O₃/ITO |
| WO₃ | WO₃/ATO | WO₃/ITO |
| CuO | XXX | CuO/ITO |
| NiO | XXX | NiO/ITO |
| SnO₂ | XXX | SnO₂/ITO |
| SiO₂ | XXX | SiO₂/ITO |
| MgO/MgF₂ | XXX | MgO or MgF₂/ITO |
| Al₂O₃ | XXX | Al₂O₃/ITO |
| ATO | — | ATO/ITO |
| ITO | ITO/ATO | — |

The photoactive constituent pairings may be pairings of simple nanoparticles, pairings of mixed metal oxide nanoparticles, and pairings of physically mixed nanoparticles.

Examples of simple nanoparticle pairings include: ZnO/TiO₂; WO₃/TiO₂; CeO₂/TiO₂; ZrO₂/TiO₂; Al2O₃/TiO₂; and pairings with metal nanoparticles such as Au, Ag, Cu, Pt and Ru or RuO₂. Examples of mixed metal oxide nanoparticles include: ZnO—CuO/TiO₂—RuO₂; ZnO—NiO/TiO₂—MnO₂; and TiO₂ or RuO₂—TiO₂ pairing with MO₂ (where M=V, Nb, Ru, Cr or Mn). Examples of physically mixed nanoparticles include: ZnO:CuO/TiO₂:CrO₂:RuO₂ and ZnO:NiO/TiO₂:MnO₂:CeO₂.

### Selection of Layer Properties

The nanoparticle layer is also designed to obtain a desired combination of optical transparency, porosity and thickness.

Optical transparency is a desirable characteristic as it enables good light penetration throughout the layer. This provides the maximum possible light absorption by the photoactive constituent nanoparticles, thereby maximizing the formation of reactive electron-hole pairs. This allows for a greater number of CO₂ reduction events and number of H₂ and/or H₂O oxidation events on the surface of the photoactive nanoparticles.

Maximizing the porosity (e.g., about 10-90%, in particular 30-50%) of the layer also helps to promote photo-driven redox reactions by providing as much accessible active surface reaction sites to the reactants (namely CO₂ with H₂ and/or H₂O) as possible. Greater porosity allows the reactants to diffuse into the porous nanoparticle layer and find as many active surface sites on the nanoparticles as possible, as well as allowing reaction products to escape/diffuse out from the nanoparticle layer.

The thickness of the nanoparticle layer will determine the total surface area and porosity of the film and hence the number of reactant molecules that can enter the pore spaces and participate in nanoparticle surface reactions with generated electron-hole pairs. As well the layer thickness also plays a role in permitting efficient electron-hole separation and preventing electron-hole recombination.

### Layer Thickness

The layers of the disclosed photoactive material have layer thicknesses selected to promote efficient charge carrier separations and heterojunction electronic band gap coupling between different nanoparticle constituents. The layer thickness is on the order of nanoscale, that is, less than a micron thick. The thickness is selected in order to maximize charge carrier separation efficiencies and to suppress recombination of generated electron-hole pairs and help improve the photoactivity of the disclosed photoactive material.

The general efficiency of the multi-layered photoactive material in capturing light to drive a photoactive reaction is dependent on the thicknesses of the constituent layers in the layered material. Typically, there is an optimal thickness for each constituent layer. These layer thicknesses affect the efficiency of separating the generated electron-hole pairs within and between the layers. For optimal efficiency of electron-hole separation in multi-layered photoactive materials, the layer thicknesses should be selected to be equal to, slightly larger (e.g., ±2-20 nm), or slightly smaller (e.g., ±2-20 nm) than the exciton (i.e., electron-hole pair) diffusion lengths. The diffusion lengths depend upon the choice of the photoactive constituents (e.g. diffusions length for Fe₂O₃ ≈20-25 nm and TiO₂ ≈27-30 nm). These diffusion lengths and optimal layer thicknesses are commonly known.¹⁶

Generally, the exciton diffusion length is dependent on exciton mobility and exciton lifetime. Exciton mobility depends on exciton diffusion lengths (e.g., the thickness of a thin film containing the exciton). Exciton lifetimes can be extended through the use of triplet semiconductor materials, which often posses much longer exciton lifetimes compared with singlet semiconductor materials.

Where the photoactive material is a single-layer arrangement of mixed constituents, the efficiency of separating the electron-hole pairs is affected by the distance between two different photoactive constituents. This distance is largely dependent on the size of the constituent nanoparticles, since the greatest distance between electron-hole pairs would be the distance between the centers of two different adjacent constituent nanoparticles. Similar to the determination of layer thickness described above, the nanoparticle size should be selected to be equal to, slightly larger (e.g., ±2-20 tun), or slightly smaller (e.g., ±2-20 nm) than the exciton diffusion lengths of the photoactive constituent nanoparticles.

Judiciously selected layer thicknesses and nanoparticle sizes results in improved gas-diffusion processes and flow-through properties, as well as contact and residence times for gas-solid photoreactions in the photoactive material.

The layers may have thicknesses in the range of about 1 nm to about 1000 nm. It has also been shown, both from literature¹⁶ and from studies discussed herein that a thinner layer (e.g., about 20-40 nm) or ultra-thin layer (e.g. no more than about 20-25 nm±8 nm) helped to improve the photoactive properties of the layer.

Although layers discussed in literature¹⁶ typically are based on dense films and not porous nanoparticle layers, experimental results discussed herein provide evidence that even thinner porous layers provide better performance than dense films.

### Layer Porosity

As discussed above, greater porosity in the layers allows for greater gas permeability and thus greater access of reactant gases to catalytic nanoparticle surfaces but may be less surface area; on the other hand, less porosity in the layers may lead to greater surface area for catalysis to occur, but with less permeability and longer contact/residence times inside the photoactive layers. This trade-off in porosity may be selected in order to obtain a desired gas diffusion rate, permeability, gas contact time, flow rate etc., and for example may be also varied through the layer thickness and porosities caused by variation in nanoparticle sizes and/or the layer arrangement or architectures of the employed material.

Porosity in the layer may also allow for an effect known as the “antenna effects of charge carrier transfer”^{13,17}. The antenna effect allows charge carriers (i.e., holes or electrons) to be transported over many different particles as well as located at distinct particles for redox processes, thereby improving photoactivity.

It has been found¹⁸ that porosity of a given nanoparticle layer is based on mass. Porosity of a layer can be measured through physisorption measurements in terms of specific porosity (cc/g), pore size (nm) and surface area (m²/g). For example the measured surface area of different sized Fe₂O₃ and ZnO nanoparticles (ranging from about 3 nm to about 47 nm in diameter) and for different layer thicknesses (ranging from about 57 nm to about 107 nm) has been found to be dependent on nanoparticle size and to be in the range of ≈30 to 242 m²/g. Specific porosity for Fe₂O₃ and ZnO nanoparticles were found to be in the range from 0.100 to 0.400 cc/g.

Other experimentally determined porosities, using EP measurements, for different nanoparticle layers are shown in the table below:

| | Porosity | Porous Layer Thickness |
|---|---|---|
| Nanoparticle | (relative humidity | (nm) (determined by |
| constituent | 0 to 100%) | SEM cross section) |
| TiO₂ | 38 | ~90 |
| WO₃ | n.d. | ~55 |
| ZnO | 43 | ~110 |
| Fe₂O₃ | 28 | ~80 |
| CuO | 52 | ~70 |
| Al₂O₃ | 34 | ~140 |
| SiO₂ | 47 | ~120 |

In the above examples, the porosity was measured in the range of about 30 to about 50%, based on condensed water within the pores of the porous nanoparticle layers, as determined by EP measurements. It should be understood that greater or lower degrees of porosity can be obtained, for example as low as 10% or lower, or as high as 90% or higher, using suitable methods. As discussed above, porosity can be controlled through control of nanoparticle sizes, nanoparticle surface area, hydrophilic and hydrophobic surface groups on the nanoparticles, as well as from various thermal treatment processes (e.g. calcination sintering effects).

### Single-Layer Photoactive Material

The present disclosure describes single-layer photoactive materials.

A single-layer photoactive material includes a mixture of two or more photoactive constituents that together participate in a photoreaction. The constituents are nanoparticles having a size that can be selected to enable the photoactivity described above. The selection of constituents and design of layer thicknesses will be described in further detail below.

The single-layer photoactive material may be fashioned as a nanoparticle optically transparent thin film having a controlled degree of porosity. These structures may be mechanically flexible (e.g., in the form of a thin film or a membrane).

The photoreaction occurring with a single-layer photoactive material is now described. For simplicity and generalization, the photoactive constituent nanoparticles in the material will be referred to as np(1) and np(2). The VB, CB and Eg values of np(1) and np(2) are selected, as described above, and known.

The single-layer is made of at least close packed constituent nanoparticles np(1) and np(2). Control of the layer packing is based on a colloidally stable mixed nanoparticle dispersion, which is established by control of surface charge of the nanoparticles and pH of the solution. The single-layer mixed composition nanoparticle layer is made by colloidal co-assembly of the mixed dispersion. The resultant layer has a random distribution of np(1) and np(2). This can be shown by electron microscopy elemental mapping of individual np(1) and np(2) nanoparticles. The uniform mixed nanoparticle layer is also referred to as a homogenous mixed composition np(1)|np(2) film. The thickness of the single-layer can be controlled by controlling the concentration of nanoparticles in the colloidal dispersion used in a spin-coating EISA manufacturing and calcination process. The layer thickness affects the amount of absorption of incident light, as well as the amount and diffusive transport of reactants into and out of the layer. The ratio of np(1) to np(2) may be selected to be any value, for example ranging from 1:99 to 99:1 and any values in between.

In a mixed single-layer photoactive material composed of a random distribution of close-packed nanoparticles np(1) and np(2) there will be contact areas where neighboring nanoparticles touch. Where this contact is between two different nanoparticles, the contact is referred to as a heterojunction in the fields of solid state chemistry and physics.

The relative energy values of the VB and CB, and size of the Eg, as selected by the choice of the nanoparticle compositions, controls the direction that electrons and holes generated in the respective touching nanoparticles will transport, separate and/or flow between the different photoactive constituent nanoparticles.

Electronic band energy alignment and band gap energies of VB and CB of np(1) relative to np(2) is chosen based on known values and measurements (e.g., X-ray photoelectron spectroscopy (XPS), ultraviolet photoelectron spectroscopy (UPS) and spectroscopic measurements). As explained above, these energy values affect the direction of transport of generated electrons and holes across the heterojunction. In this example, assuming that the VB and CB values of np(1) is lower than that of np(2) (e.g., as in FIG. 1), the electrons will travel to np(1) and the holes will travel to np(2).

The generated exciton has a known diffusion length which controls the distance over which the electron and hole can separately travel, to participate in reactions rather than deleterious recombination.

The reactants diffuse into the high surface area pore spaces in the nanoparticle layer and adsorbs on the surface of the photoactive nanoparticles. When electrons and holes are generated through photoreactions, the following redox reaction can occur:

CO₂ reduction by electrons in np(1) and H₂ or H₂O oxidation by holes in np(2)

This redox reaction can be controlled to selectively generate desired fuels, such as hydrocarbons and oxygenated hydrocarbons, in particular methane or methanol. Selectivity of the reaction can be controlled largely through the choice of the nanoparticle composition. Other factors that may contribute to selectivity may include alignment of electronic band energies and band gaps, surface area of the nanoparticle layer, porosity of the layer, thickness of the layer, absorption strength, scattering-reflection-transmission losses, electron-hole diffusion length, as well as the presence of co-catalytic compositions and various other additives.

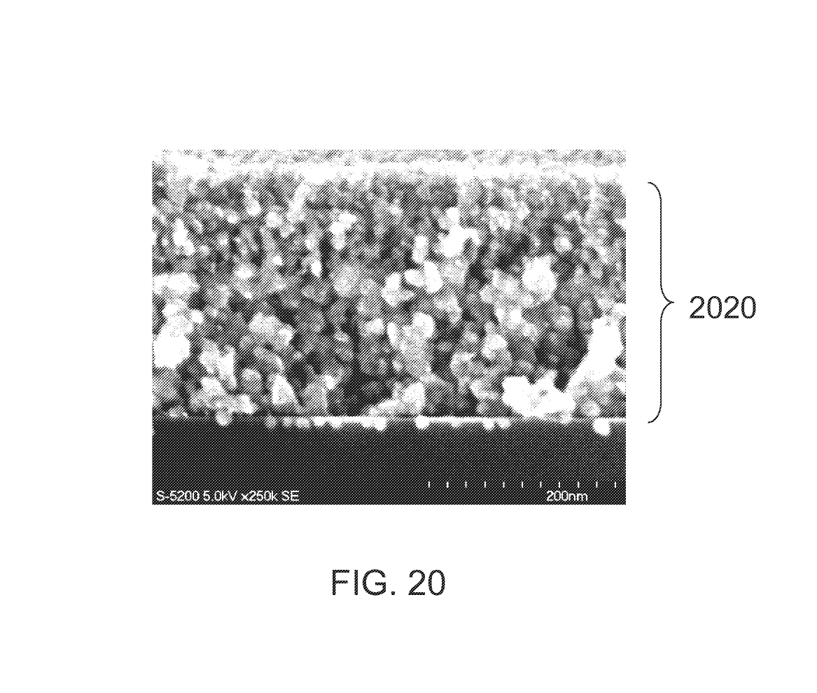

FIG. 20 show an example single-layer photoactive material 2020 having Fe₂O₃ nanoparticles mixed with CuO nanoparticles in a porous thin film layer.

Although the single-layer photoactive material has been described above as having a mixture of np(1) and np(2) in a single layer, it should be understood that the single-layer photoactive material may include further additives and/or photoactive constituents. For example, the single-layer photoactive material may include a mixture of nanoparticles of three or more different photoactive constituents.

### Multi-Layered Photoactive Material

The present disclosure describes multi-layered photoactive materials. The multi-layered material includes at least two types of layers—a first layer type having nanoparticles of a first photoactive constituent and a second layer type having nanoparticles a second photoactive constituent. The first and second layer types may be in an alternating configuration. A simple multi-layered material is a bilayer including one first layer type and one second layer type. Photoreactions can occur within each layer as well as at the interface between adjacent layers in the layer arrangements.

A difference between the single-layer photoactive material described above, in which at least two different photoactive constituents are mixed within the same layer, and the multi-layered photoactive material, in which different photoactive constituents are arranged in separate layers, is that the heterojunction contacts in the former are between nanoparticles in the same single layer whereas in the latter the heterojunction contacts are made by the nanoparticles in contact at the interface or boundary between adjacent layer planes. So, where the multi-layered photoactive material contains N number of layers, the number of heterojunctions is N−1.

Since the constituent nanoparticles are selected, as described above, to have certain VB, CB and Eg values, the heterojunction contact between adjacent photoactive nanoparticle layers determine the direction of charge flow of the generated electron and hole pairs across the interface between adjacent nanoparticle layers. Thus, the more interfaces between layers, the more separated electrons and holes are generated to take part in chemical reactions in the adjacent layers; the greater the number of layers the better the chance for these processes to occur.

The thickness and arrangement of the layers are designed to help optimize the reactions with light and the efficiency of the separation of the generated electrons and holes in order to maximize their reduction and oxidation reactions.

For simplicity and generalization, the following description will refer to the photoactive constituent nanoparticles of the multi-layered photoactive material as np(1) and np(2). An example photoactive material is composed of layers of np(1) alternating with layers of np(2). At minimum, there should be at least one layer of np(1) and at least one layer of np(2). While there is no theoretical maximum number of layers, optical transparency of the material may suffer when a very large number of layers (e.g., 20 or 100) are used.

Consider now a bi-layer comprising one np(1) layer and one np(2) layer. Heterojunctions are created between the np(1) and np(2) that are in contact at the interface between the np(1) layer and np(2) layer. Reactions at these heterojunctions are controlled by the values of the respective VB, CB and Eg of the np(1) and np(2) in contact at the interface between the two layers. The relative positions, magnitudes and alignments of the VB, CB and Eg determine the direction of flow (i.e., vectorial transport) of the electrons and holes generated in response to incident light. The vectorial transport of electrons and holes between np(1) and np(2) determines the layer in which the reduction (of CO₂) and oxidation (of H₂ and H₂O) reactions occur to generate fuel products.

As explained above, the thicknesses of the individual layers in the multi-layered structure relative to the exciton diffusion length controls the effectiveness of separating the generated electron-hole pair and the efficiency of getting them to undergo redox reactions before any counterproductive electron-hole recombination reactions occur. The exciton diffusion lengths of different nanoparticle species are generally known¹⁶, and are typically in the range of about 2-1000 nm, in particular about 10-50 nm. In general, the layer thickness should be selected to be equal to or only slightly greater or less (e.g., no more than 2-20 nm greater or less) than the exciton diffusion lengths of the respective nanoparticle species.

Different layers in the multi-layered photoactive material may have different optical thicknesses, which is defined as the refractive index of the layer times the layer thickness. The optical thicknesses of the layers, which may exhibit distinct absorption properties, can be controlled, using known techniques¹⁹, to enable photoreactions at certain wavelengths or wavelength range (e.g., ultraviolet, visible, near infrared), including broadband sunlight.

The multi-layered photoactive material may be fashioned as a nanoparticle optically transparent thin film having a controlled degree of porosity. These structures may be mechanically flexible (e.g., in the form of a thin film or a membrane).

The multi-layered photoactive material may include, as one or more of its layers, one or more mixed single-layer photoactive materials. A plurality of mixed single-layer photoactive materials may also be combined to form a multi-layered photoactive material. Although the above description refers to at least one layer of np(1) alternating with at least one layer of np(2), it should be understood that either one, or both, of the np(1) and np(2) layers can include additional photoactive constituents and/or additives. For example, the multi-layered photoactive material may include at least one layer of np(1) alternating with at least one layer of np(2)/np(3), where np(3) are nanoparticles of a third photoactive constituent. In this way, a multi-layered photoactive material may include the single-layer photoactive material, which is described above.

In some examples, two or more mixed single-layer photoactive materials having the same constituent nanoparticles but different porosities can be combined to form a multi-layered photoactive material in which the constituents are the same throughout but the porosity is different between different layers. In other examples, two or more single-layer photoactive materials having different constituent nanoparticles can be combined to form a multi-layered photoactive material.

The arrangement of the constituent layers may be periodic or aperiodic. These layers may be organized to create homo-structures (i.e., A-A), in which the layers have the same constituents but different porosities; or hetero-structures (i.e., A-B), in which the layers have respective different constituents with the same or different porosity. The layers may also have gradient arrangements (i.e., increasing change of a property along sequential layers) or tandem arrangements (i.e., two or more multi-layered structures are superimposed together). In such arrangements, the layers may be configured to exhibit a “cascade” effect, in which sequential layers or blocks of layers in the photoactive material absorb sequential wavelengths of light.

A multi-layered photoactive material may include a lattice (e.g., as in a photonic crystal) fabricated from alternating nanoparticle layers having a 1D periodicity and with selected and specified photoactivity. The selection of the constituents and photoactivity will be described in further detail below.

The multi-layered photoactive material may also be configured as a tandem and/or gradient assembly of a predetermined number of single-layer, bi-layers and/or multi-layers. The multi-layered photoactive material may have a structure and redox functions mimicking the 1D periodic thylakoid membrane stacks of the natural leaf.

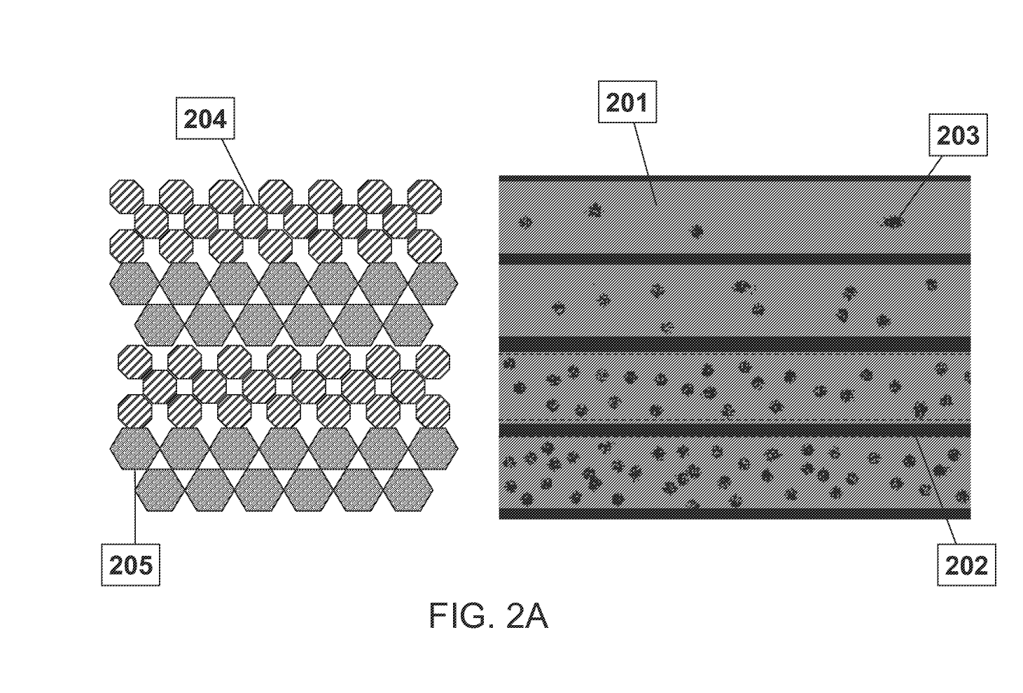

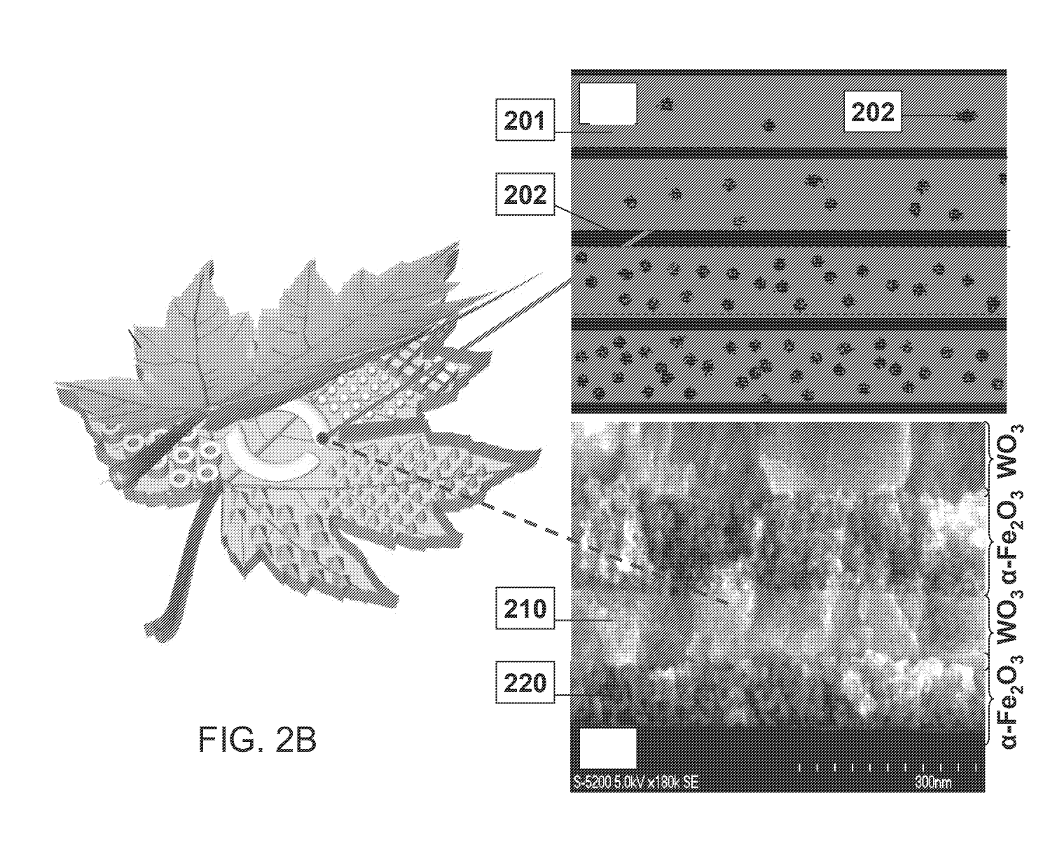

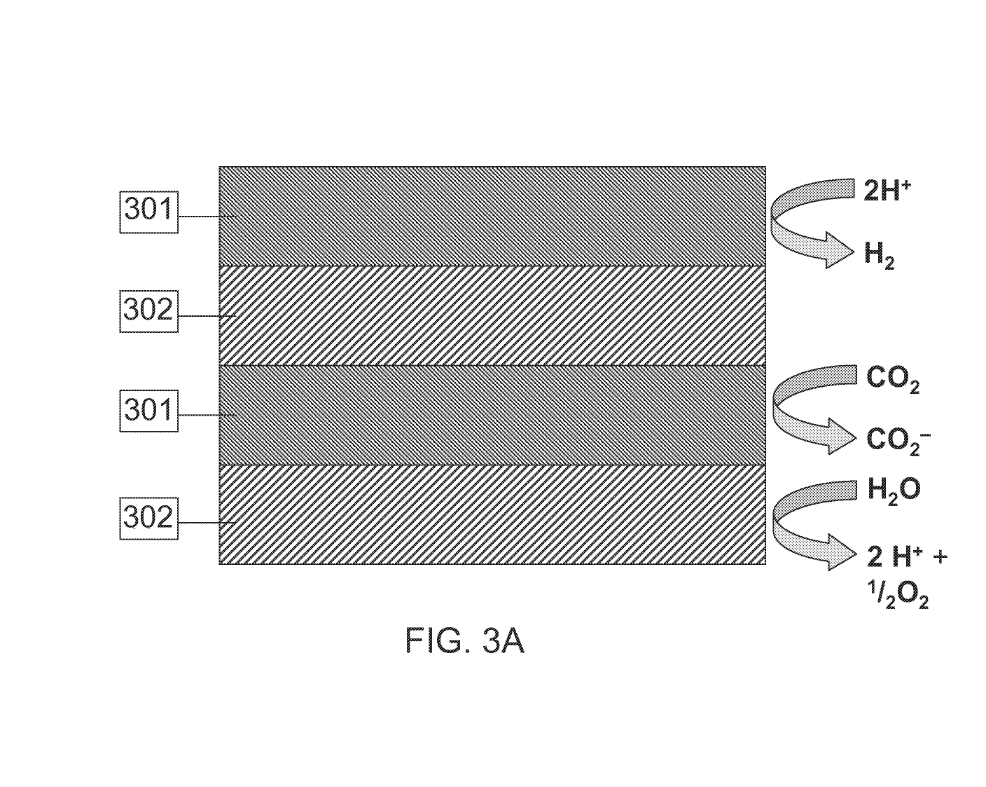

FIGS. 2A and 2B provide comparisons of example multi-layered photoactive materials with the thylakoid membrane stacks of a natural leaf. A leaf's structure includes a double-lipid membrane 201 having high refractive index (RI), a separating H₂O/electrolyte layer 202 with low RI, and embedded photosynthetic pigment proteins or molecules 203 (e.g., chlorophyll or porphyrin). In comparison, an example multi-layered photoactive material includes, for example, first metal oxide or semiconductor porous layers 204 having high RI photoactive constituents alternating with, for example, second metal oxide/semiconductor porous layers 205 having low RI photoactive constituents. Similarly, the leaf's structure is comparable to an example multi-layered photoactive material including WO₃ layers 210 having high RI photoactive constituents alternating with Fe₂O₃ layers 220 having low RI photoactive constituents. The two electrically coupled photoactive constituents may be considered to behave analogously to the biological coupling of photosystems PSI and PSII in the thylakoid membrane stack of the natural leaf.

The multi-layered photoactive materials in the examples of FIGS. 2A and 2B are porous nanoparticle multilayer architectures having a 1D periodicity.

### Photonic Structure of Multi-Layered Photoactive Materials

The multi-layered photoactive materials may be arranged to exhibit a photonic structure with a 1D periodicity. By 1D periodicity, it is meant that the layers in the photoactive material alternate in a periodic manner. By photonic structure, it is meant that the layers have a periodicity optical thickness that give rise to a photonic effect in response to incident light¹⁹. A photonic structure gives rise to a photonic band gap in the transmission spectrum of the material, in which light having wavelengths within the photonic band gap is reflected from the material.

In order to achieve a structure with good photonic crystal behavior, the RI contrast between layers should be high, as discussed above. Known RI values can be found in various references and databases¹⁴. Some examples are shown in the table below:

| Nanoparticle constituent | Refractive Index (RI for n_{λ633}) |
|---|---|
| TiO₂ | 1.65 (anatase); 1.82 (rutile) |
| WO₃ | 2.05 |
| ZnO | 1.45 |
| Fe₂O₃ | 1.35 |
| CuO | 1.29 |
| Al₂O₃ | 1.34 |
| SiO₂ | 1.31 |

Using these examples, RIC for various constituent pairs can be calculated as follows:

RI(WO₃ with 2.05)−RI(ZnO with 1.45)=RIC 0.6 1)

RI(WO₃ with 2.05)−RI(Fe₂O₃ with 1.35)=RIC 0.7 2)

RI(TiO₂/rutil with 1.82)−RI(CuO with 1.29)=RIC 0.53 3)

Experimentally, it has been shown that a photonic band gap arises in a multilayered material if the RIC is above >0.3.

In the present disclosure, a photonic structure in a multi-layered photoactive material may arise from a measurable difference in refractive index between the constituent layers. For example, a difference in refractive indices can arise from differences in thicknesses (e.g., anywhere between 1 nm and 100,000.00 nm or greater thickness), differences in layer or multi-layer porosity, differences in bulk and/or surface composition, and/or differences of any combination of the aforementioned characteristics. The optical thickness of a layer largely controls the wavelength of the photonic stop band, the wavelengths of the photonic stop band edges, and electronic absorption strength of the layer.

For photonic multi-layered photoactive materials, selection of geometrical and refractive index differences allows for control (or “tuning”) of the widths of the photonic band gap. The band gap may be tuned to have a width anywhere in the range of 1 nm to 100,000 nm, for example, and may be tuned to position the band gap edges anywhere in the deep ultraviolet, ultraviolet, visible, near infrared and microwave wavelength ranges, as discussed above.

The band gap of the photonic structure may also be tuned (i.e., changed in wavelength position, width and/or transmissivity) through an external stimulus (e.g., changes in temperature, pressure, humidity, external mechanical force, external electrical stimulus and infiltration or loss of solvent molecules). Examples of such tuning through an external stimulus are known¹⁹.

### Effect of Slow Photons

Photoactive photonic materials may exhibit trapped or localized light (which phenomenon may also be referred to as “slow light” or “slow photons”).²⁰ The effect of slow photons within a photoactive photonic lattice has been described in U.S. provisional patent application No. 61/381,656 and is generally known in the context of 3D periodic photonic crystals²¹.

In materials structured as photonic crystals, the term slow photons may be used to describe light with reduced group velocity²⁰, which may be a means to increase the effective optical path length of light in a photonic crystal, namely a periodic dielectric structured in 1, 2 or 3D with respective lattice dimensions fashioned at the wavelength of light.

Slow photons may occur in photoactive photonic crystals comprised of multi-layers made of nanoparticles. The slow photon effect occurs at wavelength ranges corresponding to the high and low energy edges of the photonic stop band as well as in resonance cavity modes. The photonic stop band reflection of a photonic crystal depends on the length scale of periodicity and/or the magnitude of the refractive index contrast within the photonic crystal. At wavelengths corresponding to the band edges of these photonic stop bands and/or resonance cavity modes, photons propagate with strongly reduced group velocity (v_{g}) as Bragg standing waves, hence they may be called “slow photons.” Thus, the group velocity for light in the photonic lattice may be very low, for example close to zero or at zero (i.e., v_{g}=0), at or near the band edges of the photonic stop band and/or at resonance cavity modes of the photonic crystal. This helps to increase the probability of absorption of the light by increasing the amount of time the photon is in the material, which in turn amplifies the photon-driven generation of electrons and holes to be utilized, for example, in the synthesis of energy-rich fuels, in particular hydrocarbons and/or oxygen-rich hydrocarbon compounds.²²

Thus, due to the slow photon phenomenon, the interaction time of light with components of the photonic lattice (for example, molecules, dyes, polymers and nanoparticles) is increased. In the case of photoactive constituents, slow photon amplified optical absorption may be achieved.

### Layer Arrangements

Some embodiments of the multi-layered photoactive material may be considered to be a biomimetic analogue of the redox-active membrane arrangement in the photoactive thylakoid multi-layer membrane ultra-structure occurring in natural leaves, as discussed above.

As described above, some embodiments of the multi-layered photoactive material may be arranged as a photonic crystal with a 1D periodicity, which may be referred to as a Bragg mirror configuration. In other embodiments, the alternating layers of the photoactive material may not form a photonic crystal structure. For example, the layers may be too thin or the refractive index difference between layers may be too low to give rise to observable photonic crystal effects, as will be described below.

Different layer thicknesses and arrangements may give rise to multi-layer interference effects (e.g., Fabry-Perot) which can enhance the absorption properties of a multi-layered photoactive material, thereby resulting in increased photoactivity.

Fabry-Perot fringes affect light absorption in various ways. Fabry-Perot fringes or interferences arise from light interaction with the nanoparticle layer, and is dependent on the layer thickness. For example the Fabry-Perot effect has been shown to constructively interfere with Au surface plasmon resonance (SPR) in the range of 450 to 650 nm to result in 10-12 times amplification of light absorption²³. The Fabry-Perot effect has also been shown to interact with back-reflecting and back-scattering layers (described further below). Similar to the achievement in enhancing light absorption in Si-based photovoltaic devices²⁴, a back-reflecting or back-scattering layer would enhance absorption peaks associated with constructive Fabry-Perot resonance modes. The Fabry Perot effect can also provide constructive interference through resonant plasmonic slits. These slits efficiently concentrate electromagnetic energy into a nanoscale volume of absorbing material placed inside or directly behind the slit. This arrangement has been found to give rise to absorption enhancements of nearly 1000%²⁵.

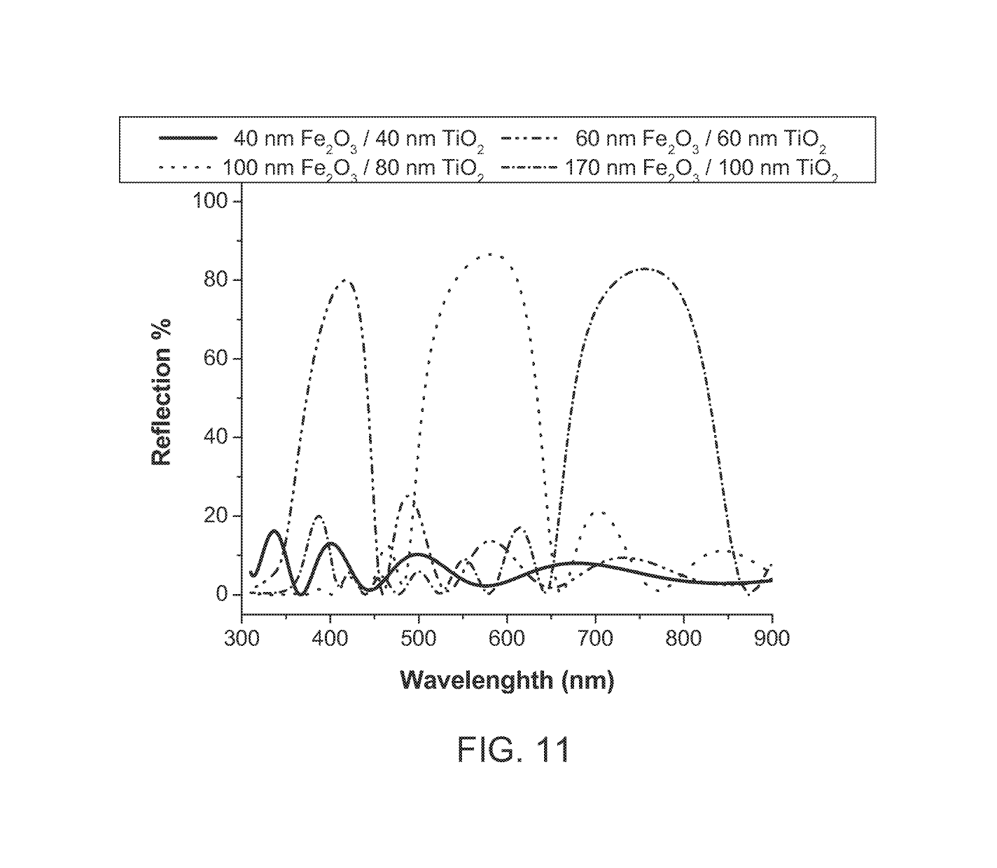

For example, FIG. 11 illustrates the reflection spectra for different multi-layered photoactive materials having different layer thicknesses. The examples shown range from one having an observable photonic stop band in the solar spectral wavelength range (namely, a material having 60 nm thick Fe₂O₃ layers alternating with 60 nm thick TiO₂ layers) to one having no detectable photonic stop band in this wavelength range (namely, a material having 40 nm thick Fe₂O₃ layers alternating with 40 nm thick TiO₂ layers). Materials with layer thicknesses between these values exhibit photonic stop bands in other wavelength ranges, though for very thin layers (e.g., ≈20-40 nm thick), the MC is too small to give rise to an observable photonic stop band effect. For example, a material having 100 nm thick Fe₂O₃ layers alternating with 80 nm thick TiO₂ layers exhibit a photonic stop band in the visible spectrum; while a material having 170 nm thick Fe₂O₃ layers alternating with 100 nm thick TiO₂ layers exhibit a photonic stop band in the near infrared spectrum.

Although the single-layer and multi-layered photoactive materials are described separately, it is possible to incorporate a mixed single-layer into a multi-layered photoactive material.

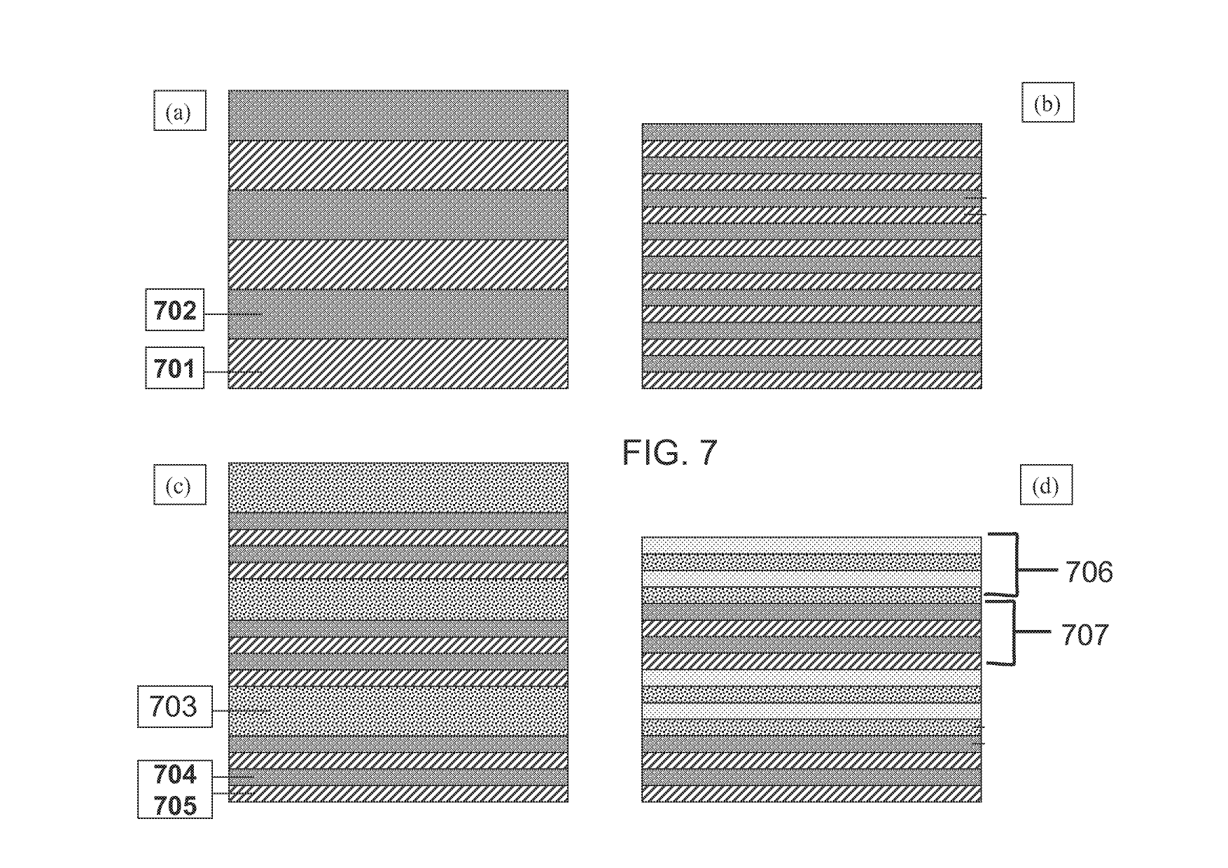

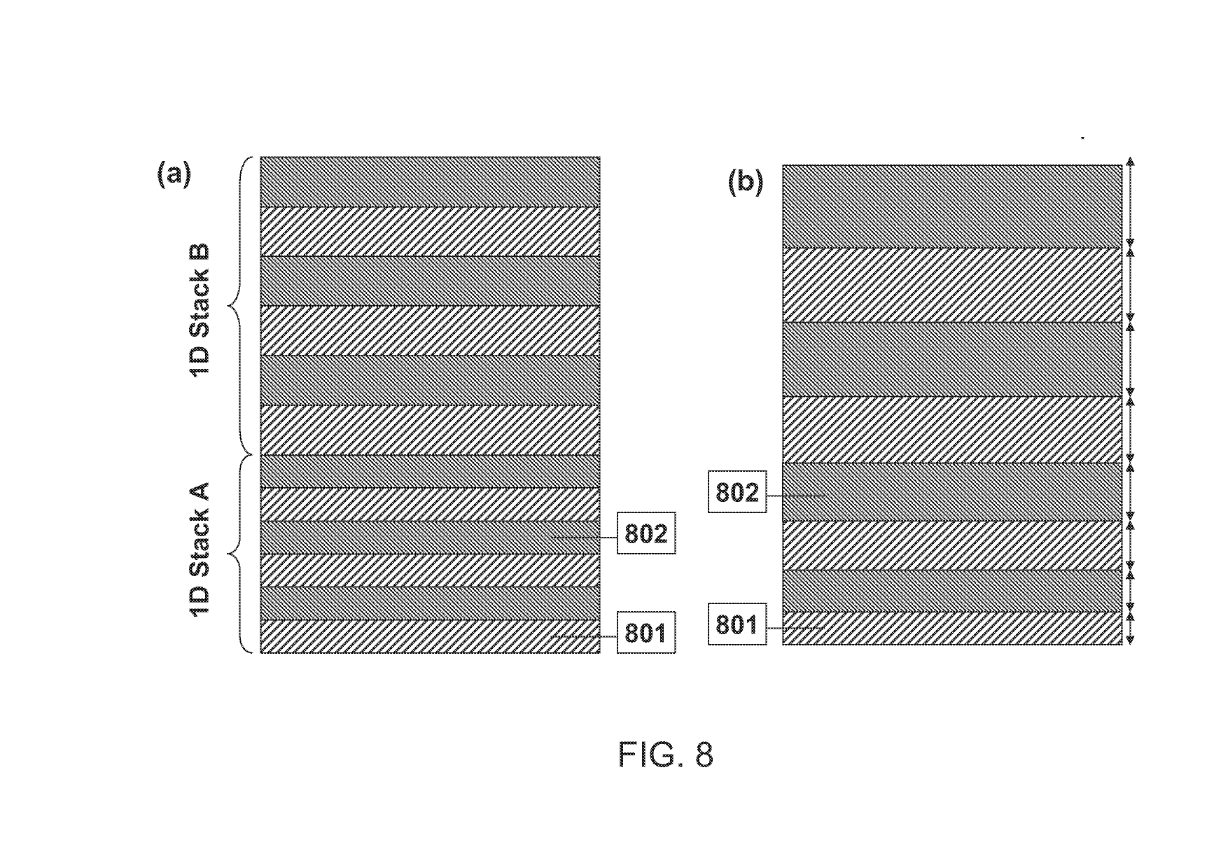

FIGS. 7 and 8 illustrate variations in the architecture of multi-layered photoactive materials and assemblies that combine two or more photoactive materials.

The architecture shown in FIG. 7A is based on a multi-layered photoactive material with micron-scale thick layers, in this example micron-scale thick layers 701 of a first photoactive constituent alternating with micron-scale thick layers 702 of a second photoactive constituent. These layers 701, 702 are arranged to form a photonic crystal structure and exploit slow photon effects.

The structure of FIG. 7B include layers with nanometer scale thickness that may be comparable to exciton diffusion lengths of the photoactive constituents. For example, these layers may be ultrathin porous single- or mixed-constituent layers. This layer arrangement helps to improve vectorial charge transport and electron hole charge separation.

The structure in FIG. 7C is a tandem photoactive material including both micron-scale thick layers 703 (which may be single- or mixed-constituent layers) and nanometer scale thick multi-layers 704, 705, which may combine the effects of both the examples of FIGS. 7A and 7B, to exploit both slow photon and exciton diffusion length effects.

The structure in FIG. 7D is a tandem photoactive material assembly combining different photoactive materials 706, 707. Each photoactive material 706, 707 includes different nanometer and/or micron scale thickness layers, and/or has different photoactive constituents. Such an assembly of two or more arrangements having different photoactive constituent pairs may help to expand the wavelength range over which photoreactions may occur. The different layer thicknesses and different constituents allow for slow photon amplification and exciton generation, vectorial charge transport and electron hole charge separation to occur in different wavelength regions of the incident solar light. As well, such photoactive material assemblies can combine two or more photoactive materials that carry out redox reactions with different reactants, in order to provide a single assembly that carry out different reactions, for example purification of different pollutants.

FIG. 8A shows an example of a photoactive material assembly combining two photoactive materials in tandem. In this example, thicker layers of photoactive constituents 801, 802 are stacked on top of thinner layers of the same photoactive constituents 801, 802. FIG. 8B shows an example of a photoactive material having layers of photoactive constituents 801, 802 that gradually (e.g., constantly or variably) decrease (or increase) in thickness. Such variations in layer thicknesses help to expand the wavelength ranges over which photoreactions may occur.

The photoactive material may be a non-planar surface, such as a cylindrical or spherical surface. Even when manufactured to be flexible, non-planar, as flakes or powder, for example, the layered structure of the multi-layered photoactive material is maintained.

### Manufacture

Methods for manufacturing the disclosed photoactive material are now described. The methods disclosed herein may be suitable for manufacture of the single-layer photoactive material as well as the multi-layered photoactive material, as described above. Variations and modifications may be made, as would be understood by a person skilled in the art.

Methods for manufacture may be based on a bottom-up approach, for example using nanoparticle colloidal assembly, as well as a top-down approach, which may be scalable for manufacturing larger photoactive materials, as will be described below. The methods for manufacture disclosed herein may be used to manufacture photoactive materials for solar panels or photoreactors, membranes and various coatings for applications such as the large or small scale production of fuels, water-splitting applications, air and water purification as well as anti-smog solutions.

### Colloidal Suspension

A method of manufacture begins with a colloidal dispersion of the constituent nanoparticles in a solvent. The synthesis of such a colloidal suspension is generally known²⁷ and is based on choosing the nanoparticle precursor(s) and transforming the precursor(s) into nanoparticles with a selected size, shape and surface through a nucleation and growth synthesis process. The composition of the precursor(s) is selected based on the desired composition of the nanoparticles. The precursor(s) can include metals, metal alloys, metal oxides, metal sulfides, metal carbides and any photoactive semiconductor materials, among others. The size of the nanoparticles is controlled in the nucleation and growth process by controlling the conditions during synthesis. The nanoparticle sizes can be in general controlled to range in diameter from 1 nm to several microns. The surface charge of the nanoparticles can also be controlled by controlling the conditions used to synthesize the nanoparticles and the solvent in which they are dispersed, as well as the pH and/or the ionic strength of the resulting solution (e.g., by adding salts and/or buffer additives).

The stability of the colloidal suspension is also important to allow manufacture of high quality films with a selected thickness and porosity. The principles of colloidal stability are generally known²⁷ and are based on the different kinds of forces between the suspended nanoparticles, as determined by the nature of the surfaces of the nanoparticles.

In this particular application for manufacturing photoactive materials, the nanoparticles used are mostly provided as charge-stabilized colloidal suspensions, where the electrical double layer (EDL) forces and the Van der Waals (VDW) forces are balanced such that the nanoparticles are kept separate, dispersed and suspended in the colloidal suspension.

Examples of nanoparticle composition selection and dispersion are described in literature^{18,26}. Examples include sol-gel synthesis of ZnO and Fe₂O₃ or TiO₂ nanoparticles ranging in size from ≈3 to 50 nm in diameter, as well as other non sol-gel based synthesis of metal-oxide nanoparticles, such as WO3, MoO₃, Fe₂O₃, ZnO, SnO₂ in binary and ternary form and TCOs such as ATO (Sb:SnO₂) and ITO (Sn:In₂O₅) metal oxides in the range of ≈3 to 12 nm in diameter.

Concentrations of nanoparticles in the dispersion are dependent on the amount of used precursor, which is mostly in the gram range. The resulting dispersions typically have concentrations ranging from 1 to 35 wt. %. Dilution of this dispersion can be carried out to obtain a desired layer thickness.

The nanoparticles obtained in the examples of solvent-based techniques shown in the literature typically have spherical or sphere-like dimensions. In such examples no surfactant is needed as stabilization of the dispersion occurs through surfaces charges, which can be determined through zeta-potential measurements.

In order to manufacture a single-layer photoactive material, in which two photoactive constituents are mixed within a layer, the colloidal suspension includes the two different constituent nanoparticles uniformly mixed and suspended in a selected ratio, as described above.

### EXAMPLES

The following examples describe various nanoparticle colloidal suspensions. Such suspensions have been found to be favorable for use in bottom-up sol-gel spin coating processes^{6,26}.

### Example 1

Fe₂O₃ nanoparticles were synthesized by dissolving Fe(NO₃)3.9H2O (5.05 g, 12.5 mmol) in 80 mL ROH, with R=Me, Et, n-Pr, iso-Pr, or tert-Bu, followed by addition of 20 mL deionized water (0.056 μS/cm). The resulting dark-red solution (pH≈1-2) was stirred for 12 h at room temperature (RT). The resulting orange-brown Fe₂O₃ dispersion was stored at RT in air.

### Example 2

Fe₂O₃ nanoparticles were synthesized by simple dissolution of 3 g of the elemental Fe metal powder (mesh 100 or 325), dispersed in 10-15 ml of deionised H₂O (0.056 μS/cm) followed by the addition of 10-35 mL H₂O₂ (30%. p.a.) and 3 mL of AcH (glacial acid) (ratio 40:1) at 0° C. in an ice-bath under air and further stirring for 3 day under RT, no inert atmosphere (i.e., nitrogen) needed. Since this is a very exothermic reaction, instant ice-bath cooling is necessary in a well ventilated hood.

### Example 3

TiO₂ (rutile form) nanoparticles were synthesized using 18.75 mL of Ti(OiPr)₄ Titanium-iso-propoxide added dropwise under vigorous stirring at RT to 110 mL of an aqueous 0.1 M nitric acid (HNO₃) mixture. The resulting slurry was heated at 80-90° C. for an additional 8 hours, the resulting white-milky TiO₂ dispersion was cooled down to RT and the dispersion was stored at RT in a brown glass vessels for further use.

### Example 4

TiO₂ (anatase form) nanoparticles were synthesized using 17 mL of Ti(OiPr)₄ Titanium-iso-propoxide added dropwise under vigorous stirring at RT to 80 mL of MeOH. After addition of 2 mL of AcH and ≈1-2 ml of distilled water the resulting slurry was heated at 80-90° C. for an additional 8 hours, the resulting white-milky TiO₂ dispersion was cooled down to RT and the dispersion was stored at RT in a brown glass vessels for further use.

### Example 5

Sb:TiO₂ (anatase form) nanoparticles were synthesized using 17 mL of Ti(OiPr)₄ Titanium-iso-propoxide added dropwise under vigorous stirring at RT to a mixture of 80 mL of MeOH with dissolved Sb(OAc)₃ 30-50 mg (0.1 to 0.170 mmol). After further addition of 2 mL of AcH and ≈1-2 ml of distilled water the resulting slurry was heated at 80-90° C. for an additional 8 hours, the resulting yellow-milky TiO₂ dispersion was cooled down to RT. The dispersion was stored at RT in brown glass vessels for further use.

### Example 6

ZnO(O₂) nanoparticles were synthesized by simple dissolution of 3 g (45.89 mmol) of the elemental Zn metal powder (mesh 100 and 325), dispersed in 10-15 ml of deionised H₂O (0.056 μS/cm) followed by the addition of 10-35 mL H₂O₂ (30%. p.a.) and 3 mL of AcH (ratio ≈10:1) at 0° C. in an ice-bath under air, and further stirring at RT overnight, no inert atmosphere (i.e., nitrogen) needed. Since this is a very exothermic reaction, instant ice-bath cooling is necessary in a well ventilated hood.

### Example 7

WO₃ nanoparticles were synthesized by dissolution of elemental W powder (ASP powder 1-5 μm or mesh 325) 5.53 g (30.1 mmol) in 50 mL of H₂O₂ (30% p.a.) and 5 mL of AcH (ratio ≈10:1) at 0° C. by cooling the reaction mixture with an ice-bath. The exothermic dissolution/oxidation process leads to a light-yellow WO₃ dispersion under air. This was further stirred at RT overnight, no inert atmosphere needed, and was stored in a plastic bottle at 4° C. Since this is a very exothermic reaction, instant ice-bath cooling is necessary in a well ventilated hood.

### Example 8

CuO nanoparticles were synthesized using a solution of ≈0.300 mL with 2.5 g of Cu(OAc)₂ was mixed with 1 mL of AcH and heated under reflux with vigorous stirring up to 110° C., then about 0.8-1 g of solid NaOH pellets (p.a. grade) was instantly added to the boiling mixture. A large amount of black-precipitate was directly produced, the mixture was cooled to RT, the obtained dark-black precipitate was centrifuged for 5 min at 7300 rpm and additionally washed once with distilled water and three times with absolute ethanol. The resulting powder was dried in air at RT and re-dispersed in water under sonication for at least 12 h.

### Example 9

NiO nanoparticles were synthesized by dissolution of elemental Ni powder (mesh 325) 7 g (85.2 mmol) in 50 mL of H₂O₂ (30% p.a.) and 7 mL of AcH (ratio ≈10:1) at 0° C. by cooling the reaction mixture with an ice-bath. The exothermic dissolution/oxidation process leads to a greenish NiO dispersion under air. This was further stirred at RT for 5-7 days, no inert atmosphere needed, and was stored in a plastic bottle at 4° C.

### Example 10

CoO nanoparticles were synthesized by dissolution of elemental Co powder (ASP powder 1-5 μm or mesh 325) 5 g (84.7 mmol) in 50 mL of H₂O₂ (30% p.a.) and 5 mL of AcH (ratio ≈10:1) at 0° C. by cooling the reaction mixture with an ice-bath. The exothermic dissolution/oxidation process leads to a purple-red CoO dispersion under air. This was further stirred at RT overnight, no inert atmosphere needed, and was stored in a plastic bottle at 4° C.

### Example 11

MgO nanoparticles were synthesized by dissolution of elemental Mg-chips 5.0 g (205.8 mmol) in 50 mL of H₂O₂ (30% p.a.) and 5 mL of AcH (ratio ≈10:1) at 0° C. by cooling the reaction mixture with an ice-bath. The exothermic dissolution/oxidation process leads to a transparent MgO dispersion under air. This was further stirred at RT overnight, no inert atmosphere needed, and was stored in a plastic bottle at 4° C.

### Example 12

MoO₃ nanoparticles were synthesized by dissolution of elemental Mo powder (100 mesh or mesh 325) 5.0 g (52.11 mmol) in 50 mL of H₂O₂ (30% p.a.) and 5 mL of AcH (ratio ≈10:1) at 0° C. by cooling the reaction mixture with an ice-bath. The exothermic dissolution/oxidation process leads to a yellow-orange WO₃ dispersion under air. This was further stirred at RT overnight, no inert atmosphere needed, and was stored in a plastic bottle at 4° C.

### Example 13

MgCo₂O₄ nanoparticles were synthesized by dissolution of elemental Co powder (ASP powder 1-5 μm or mesh 325) and elemental Mg chips 0.24 g (10 mmol)+1.18 g (20 mmol) Co-powder dispersed in 10 mL of water and an further slow addition of 30 mL of H₂O₂ (30% p.a.) and 5 mL of AcH at 0° C. by cooling the reaction mixture with an ice-bath. The exothermic dissolution/oxidation process leads to a dark-brown MgCo2O4 dispersion under air. This was further stirred at RT overnight, no inert atmosphere needed, and was stored in a plastic bottle at 4° C.

### Example 14

MgFe₂O₄ nanoparticles were synthesized by dissolution of elemental Fe powder (mesh 100 or 325) and elemental Mg chips 0.24 g (10 mmol)+1.11 g (20 mmol) Fe-powder dispersed in 10 mL of water and further slow addition of 30 mL of H₂O₂ (30% p.a.) and 5 mL of AcH at 0° C. by cooling the reaction mixture with an ice-bath. The exothermic dissolution/oxidation process leads to a dark-red MgFe₂O₄ dispersion under air. This was further stirred at RT for 3-4 days, no inert atmosphere needed, and was stored in a plastic bottle at 4° C.

### Example 15

Fe_{0.3}CO_{0.7}MoO₄ nanoparticles were synthesized by dissolution of elemental Fe, Co and Mo powder (mesh 100 or 325) with elemental Fe powder 0.17 g (3 mmol)+elemental Co powder 0.41 g (7 mmol)+elemental Mo powder 0.96 g (10 mmol) dispersed in 10 mL of water and further slow addition of 30 mL of H₂O₂ (30% p.a.) and 5 mL of AcH at 0° C. by cooling the reaction mixture with an ice-bath. The exothermic dissolution/oxidation process leads to a brownish Fe_{0.3}Co_{0.7}MoO₄ dispersion under air. This was further stirred at RT for 3-4 days, no inert atmosphere needed, and was stored in a plastic bottle at 4° C.

### Example 16

SnO₂ nanoparticles were synthesized by dissolution of elemental Sn powder (ASP powder<10 μm) 3 g (25.3 mmol) dispersed in 5 mL of distilled water and further addition of 8 mL of HCl (37% p.a.) to etch the SnO₂-surface, resulting in compact piece of pure Sn-metal. Further slow addition of 25 mL H₂O₂ (30% p.a.) and of 5 mL of AcH under vigorous stirring leads to complete dissolution at 0° C. by cooling the reaction mixture with an ice-bath. The exothermic dissolution/oxidation process leads to a white-milky transparent SnO2 dispersion under air. This was further stirred at RT overnight, no inert atmosphere needed. The resulting dispersion was stored in a plastic bottle at 4° C.

### Example 17

(Sb:SnO₂) ATO nanoparticles were synthesized by dissolution of elemental Sn powder (ASP powder<10 μm) 2.97 g (25.0 mmol) and elemental Sb powder 10 wt. % (mesh 325) 0.3 g (2.5 mmol) dispersed in 5 mL of distilled water and further addition of 8 mL of HCl (37% p.a.) to etch the native-bare SnO₂ and Sb₂O₃ surface, resulting in compact piece of pure SnSb-metal. Further slow addition of 25 mL H₂O₂ (30% p.a.) and of 5 mL of AcH under vigorous stirring leads to complete dissolution at 0° C. by cooling the reaction mixture with an ice-bath. The exothermic dissolution/oxidation process leads to a deep bluish-transparent (Sb:SnO₂) ATO dispersion under air. This was further stirred at RT overnight, no inert atmosphere needed. The resulting dispersion was stored in a plastic bottle at 4° C.

### Example 18

ZnSnO₃, ZTO nanoparticles were synthesized by dissolution of elemental Sn powder (ASP powder<10 μm) 1.78 g (15.0 mmol) and elemental Zn powder 50 wt. % (mesh 100) 0.98 g (15 mmol) dispersed in 5 mL of distilled water and further addition of 8 mL, of HCl (37% p.a.) to etch the native-bare SnO₂ and ZnO surface, resulting in compact piece of pure SnZn-metal. Further slow addition of 25 mL H₂O₂ (30% p.a.) and of 5 mL of AcH under vigorous stirring leads to complete dissolution at 0° C. by cooling the reaction mixture with an ice-bath. The exothermic dissolution/oxidation process leads to a white-milky transparent ZnSnO₃ (ZTO) dispersion under air. This was further stirred at RT overnight, no inert atmosphere needed. The resulting dispersion was stored in a plastic bottle at 4° C.

### Example 19

In₂O₅ nanoparticles were synthesized by dissolution of elemental In powder (mesh 325) 3 g (26.1 mmol) dispersed in 5 mL of distilled water and further addition of 8 mL of HCl (37% p.a.) to etch the native-bare In₂O₅-surface, resulting in compact piece of pure In-metal. Further slow addition of 25 mL H₂O₂ (30% p.a.) and of 5 mL of AcH under vigorous stirring leads to complete dissolution at 0° C. by cooling the reaction mixture with an ice-bath. The exothermic dissolution/oxidation process leads to a light-yellow transparent In₂O₅ dispersion under air. This was further stirred at RT overnight, no inert atmosphere needed.

The resulting dispersion was stored in a plastic bottle at 4° C.

### Example 20

(Sn:In₂O₅) ITO nanoparticles were synthesized by dissolution of elemental Sn powder 10 wt. % (ASP powder<10 μm) 0.297 g (2.5 mmol) and elemental In powder (mesh 325) 2.87 g (25 mmol) dispersed in 5 mL of distilled water and further addition of 8 mL of HCl (37% p.a.) to etch the native-bare SnO₂ and In₂O₅ surface, resulting in compact piece of pure InSn-metal. Further slow addition of 25 mL H₂O₂ (30% p.a.) and of 5 mL of AcH under vigorous stirring leads to complete dissolution at 0° C. by cooling the reaction mixture with an ice-bath. The exothermic dissolution/oxidation process leads to a light yellow-greenish (Sn:In₂O₅) ITO dispersion under air. This was further stirred at RT overnight, no inert atmosphere needed. The resulting dispersion was stored in a plastic bottle at 4° C.

### Example 21

To any of the examples described above, various dispersions having the same compactable solvents can be mixed together in different amounts. For example, dispersions dissolved in H₂O/H₂O₂ can be mixed (e.g. mixing of NiO and MgCo₂O₄; WO₃ and Fe₂O₃; CuO and ZnO; CuO and ITO≡SnIn₂O₅; or Fe₂O₃ and Cu₂O). Another possibility is to re-disperse dried powder form of the nanoparticle in various ratios in existing liquid dispersion. For example, powder CuO can be dispersed in Fe₂O₃ or in ZnO dispersions. When such mixed dispersions are spin-coated and calcined, the result is a porous mixed nanoparticle layer containing the mixed components.

Further examples and details can be found in the literature^{18,26}, where the characteristics of the manufacture layers are also discussed.

In general, various methods known from the literature²⁷ can be used for the synthesis of various metal oxide dispersions and composition (including core-shell systems M₁O@M₂O or heterodimeric nanoparticle assemblies M₁O-M₂O)

The above example metal oxide dispersions were filtered through a 0.45 μm Titan 2 HPLC Filter Amber (GMF Membrane), to remove any agglomerates and subsequently diluted to the desired concentration, used for porous layered photoactive materials. The dispersions were diluted with deionized water to distinct concentrations. The dilutions were chosen to match a desired layer thickness (i.e., the thicker the desired layer, the less the dilution). The diluted concentrations ranged from about 1 wt. % to about 35 wt. % in these examples. Polyethylene glycol (PEG, [(C₂H₄O)n.H₂O], MW: 20.000 g/mol) was added and dissolved in the range of 1-20 wt % to prepare spinable dispersion forms before spin-coating.

### Introducing Additives

Additives for improving the behavior of the photoactive material, which will be described in further detail below, can be added to the colloidal dispersion before forming the nanoparticle layer.

For example, to any of the above-described dispersions, noble metal precursors can be added/dissolved within the prepared dispersions. For example, HAuCl₄.3H₂O can be added to obtain Au nanoparticles additives; AgNO₃ can be added to obtain Ag nanoparticles additives; and Cu(NO₃)₂.3H₂O can be added to obtain Cu nanoparticles additives. Mixtures of noble metal precursors can also be introduced. Such additives should be added with low concentrations, in the range of about 1-4×10⁻⁴ M to about 1×10⁻² M. After spin-coating of the respective dispersion having the noble metal precursors, SPR-active noble metals (e.g., Au, Ag or Cu) can be generated via photo-reduction and/or in-situ through thermal treatments.

Catalytic alkali and earth-alkali promoters can also be introduced as additives into the dispersion. For example, to any of the above-described dispersions, earth alkali and alkali precursors can be added/dissolved within the prepared dispersions. Such precursors include, for example, CaCO₃, KHCO₃, NaHCO₃, and LiCO₃. Such additives can also be impregnated into the dried nanoparticle layer using diluted promoter solutions (e.g., having concentrations of about 0.001-1 M). Further calcinations and heat treatments leads to their final incorporation.

### Nanoparticle Self-Assembly and Co-Assembly

Using a colloidally stable nanoparticle suspension, evaporation-induced self assembly (EISA), such as using a spin-coating bottom-up process with additional calcination, can be used to manufacture high optical and structural quality films of controlled thicknesses. Where the suspension includes two different constituent nanoparticles, the nanoparticles can self-assemble through EISA co-assembly.

Some industrial large scale production methods and processes that may be appropriate for manufacturing the disclosed photoactive materials include: sol-gel spin coating, metal oxide chemical vapor deposition (MOCVD)²⁸, spray-coating, spray pyrolysis (SP)²⁹, ultrasonic spray pyrolysis (USP)³⁰, aerosol-coating, drop-casting, doctor-blading, draw-bar, screen-printing, ink-jet-printing, atomic layer deposition (ALD), advanced gas deposition (AGD)³¹, reactive DC magnetron sputtering³², atmospheric pressure chemical vapor deposition (APCVD)³³, potentiostatic anodization³⁴ and electrodeposition³⁵, among other large scale deposition techniques known in the art.

Other suitable large scale industrial production methods may also include roll-to-roll deposition thin film technology, large surface deposition, spraying or sputtering processes, ceramic processes, pre-treatment and deposition on existing glass or solid-surfaces, electrodeposition or galvanic processes on large surface areas and panels, among others³¹.

### Examples

For the example colloidal suspensions described above, spin-coating of the nanoparticle layer was performed on a Lauriel single wafer spin processor (Model WS-400A-6NPP/LITE) at 2500-6000 rpm, 25-60 acceleration for 20-60 sec. The resulting porous nanoparticle metal oxides thin layers were calcined at 450-600° C. for 15-60 minutes.

To prepare a multi-layered photoactive material, a pair of two different nanoparticle layers were spin-coated from modified PEG-dispersions and subsequently calcined, iteratively until the desired number of layers was deposited using various nanoparticle dispersions.

### Pre-Treatments

The dried nanoparticle layers can be further pre-treated. For example, pre-treatment can result in the making of Cu₂O and Cu⁰ metal particles within the porous layer by the reduction of CuO nanoparticles. CuO nanoparticles or films can be reduced at 320° C. for 2 h under a H₂(5 wt. %)/Ar stream with a flow-rate of ≈0.5-1 mL/sec to yield pure Cu₂O particles. Further reduction of Cu₂O and/or CuO nanoparticles or films at 400° C. for 1.5 h under a H₂(5 wt. %)/Ar stream with a flow-rate of =0.5-1 mL/sec yields pure Cu-phase. Reduction-time and reduction-temperature (e.g., about 200 to about 500° C.) may vary by using different H₂/Ar mixtures ranging from 5-95% (11₂-Mixtures) to pure (i.e., 100%) H₂ gas.

### Substrate

Another suitable method of manufacture includes thin film deposition techniques, in which the photoactive constituent nanoparticles are packed, granulated, dispersed, painted, sprayed and/or dip-coated onto a substrate, such as a photoreactor, device or any other suitable application surface.

Where the photoactive material is manufactured to be flexible, for example as a free-standing thin film, the photoactive material may be provided in non-planar shapes (such as cylinders, pyramids, gratings, etchings, domes, bowls, spheres, irregular shapes, etc.) and may be configurable to conform to a target surface. The photoactive material may be manufactured in the form of a thin film or a coating on a substrate, for example. The photoactive material may be manufactured on a rigid substrate, to provide support to the photoactive material; or on a flexible substrate, to maintain flexibility.

The substrate may be any material suitable for manufacture of conventional nanoparticle layers including, for example, glass, metal, or polymers. The substrate may be transparent to maintain the optical transparency of the photoactive material.

### Examples

Examples of suitable substrates include fluorine-doped tin oxide (FTO)-coated glass, SiO₂-coated glass and Si-wafer, which are commercially available. These substrates can be pretreated and cleaned before spin-coating.

In an example, the substrate can be treated with a mixture of H₂O₂/H₂SO₄ (3:1) and H₂O₂/NH₃.H₂O (3:1) for at least 1 hour and washed after the treatment with ethanol. The Si wafers and FTO-coated glass were further treated under air plasma for at least 5 min to remove impurities and to increase the hydrophilicity of the surface.

The photoactive material may be further processed (e.g., by grinding, crushing, sonicating or milling) to produce nano- or microscopic flakes or powders. Such flakes or powders may be about 0.01-10 μm in diameter. Such flakes or powders may be mixed with a solvent to produce a paintable or sprayable form. The flakes or powder may also be used in place of conventional photoactive powders used in photoreactors (e.g., in a packed fix bed flow-through photoreactor) or as a coating material, for example. When the photoactive material is provided in flake or powder form, the layered architecture of the photoactive material is still maintained within the flake or powder granule.

Although the above example describes certain manufacture conditions, these may be varied. For example, spinning conditions may be varied, for example as follows: spin-coating time about 5 sec to 5 mins, about 5-6000 rpm with various acceleration conditions.

Calcinations may be varied by different temperatures (e.g., about 5 to 2000° C.) and through different calcination times (e.g., about 5 min to 1000 h), as well as different post-treatment procedures (e.g., oxidation/reduction processes) may be included.

Other methods of manufacture may be used, including: for example: spin-coating, dip-coating, spray pyrolysis (SP), ultrasonic spray pyrolysis (USP), spray-coating, aerosol-coating, drop-casting, doctor-blading, draw-bar, screen-printing, ink-jet-printing, reactive DC magnetron sputtering, atmospheric pressure chemical vapour deposition (APCVD), metal oxide dhemical vapour deposition (MOCVD), molecular beam epitaxy (MBE), pulsed laser deposition (PLD), oblique angle deposition (OAD), glancing angle deposition (GLAD), potentiostatic anodization and electrodeposition. The manufacturing may include two or more deposition techniques, e.g. sol gel spin coating and sputtering or CVD techniques. Any other suitable known methods may be used.

### Layer Variation

The disclosed photoactive material, whether in the single-layer or multi-layered structure, may include one or more layer variations as described below.

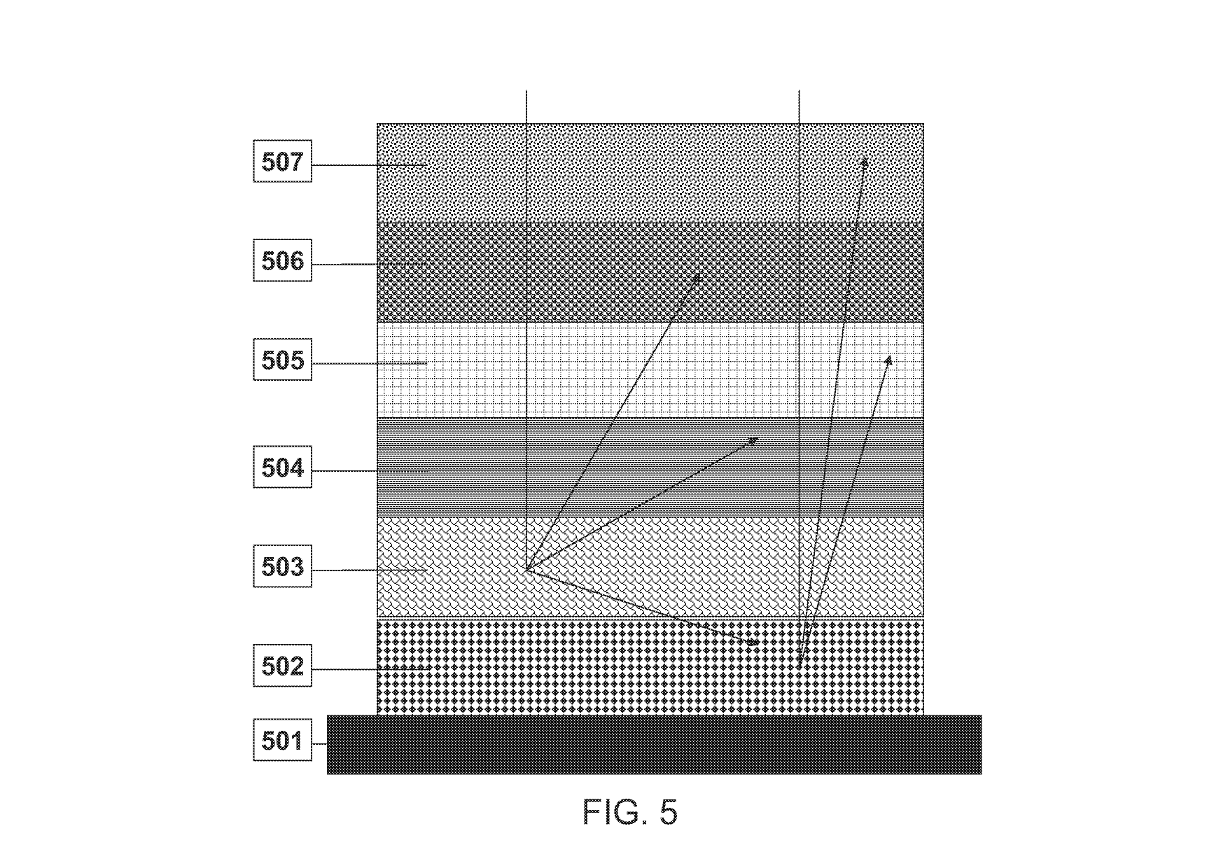

FIG. 5 is a schematic illustrating implementation of various layer variations in a photoactive material. In this example, the photoactive material includes a substrate layer 501 for supporting the material, a back-reflecting or back-scattering layer 502, a texturing layer 503, a gas-barrier layer 504, a mixed porous single-layer 505, and alternating single-constituent layers 506, 507. The scattering and reflecting of light by the back-reflecting layer 502 and the texturing layer 503 is illustrated as arrows.

Although the example of FIG. 5 shows a photoactive material having one instance of each layer variation, it should be understood that the photoactive material may have more than one instance or no instance of each layer variation. As shown in FIG. 5, in addition to the layer variations described below, the photoactive material may combine mixed single-layers 505 with alternating single-constituent layers 506, 507.

### Air or Gas-Phase Layers

The photoactive material may incorporate an air or gas-phase layer. That is, in a multi-layered photoactive material, there may be one or more spaces between layers. The presence of an air or gas-phase layer within the material may allow the gas-phase reactants (namely CO₂ and H₂ and/or H₂O) to be contained or trapped within the material, so as to be readily available to take part in the redox reaction.

### Support and Substrate Layers

The photoactive material may be manufactured as a thin film or coating on a substrate (shown as 501 in FIG. 5), wherein the substrate may be inflexible (e.g., glass, metal, ceramic) or flexible (e.g., a porous polymer substrate). The selection of the substrate material may be dependent on the desired application. For example, an inflexible substrate may be used for forming a solar panel, to be installed as part of a photoreactor or in other applications. Where the substrate is a transparent glass, the panel may be used or integrated in conventional window panel designs. Where the substrate is a ceramic, the panel may be used as a roof or facade tile. Where the substrate is a flexible membrane, the resulting photoactive membrane may be used in flow-through processes.

The following materials are examples of suitable substrate materials: SiO₂ (e.g., in the form of glass or quartz), Si-wafers, ceramic supports (e.g., SiC), porous Al₂O₃ substrates, and flexible and porous polymer substrates/membranes (e.g. Nafion). Other possible support and substrate layers include transparent conductive oxides (TCOs), and coated glass substrates with conductive layers (for example coated with e.g. ITO≡In₂O₅:Sn (Indium Tin Oxide), ATO≡SnO₂:Sb (Antimony Tin Oxide), FTO≡SnO₂:F (Flourine Tin Oxide), ZTO≡SnO₂:Zn (Zinc Tin Oxide), or IZO≡In₂O₅:Zn (Indium Zinc Oxide)).

### Internal Reflection and Scattering Layers

A light-scattering layer (shown as 503 in FIG. 5) may be incorporated into the material. Such light-scattering may also be referred to as texturing, grating, etching or changing surface morphologies.

A back-reflecting layer (shown as 502 in FIG. 5) may also be incorporated into the material. A back-reflecting layer may be, for example, a reflecting metal layer or a Bragg mirror. The back-reflecting layer is provided on the face of the material opposite to the light-receiving face of the material. The back-reflecting layer can also serve as a substrate for the photoactive material.

The inclusion of one or more such scattering or reflecting layers helps to increase the effective optical path length of light traveling through the material, and hence increases efficiency of reaction with incident light in the photoactive material. The back-reflecting layer may serve to reflect most or all of the incident light back through the layers, thereupon effectively doubling the effective optical path length of the light in the material and thus doubling the yield of fuel products for a given amount of light.

A light-scattering layer will also help to improve light absorption. Two types of light absorption may be distinguished: (i) volume absorption, for example in a textured optical layer; and (ii) surface absorption. Based on the theory of light trapping³⁶ in scattering layers, enhancement factors of 2n² to 4n² may be expected for bulk or volume absorption of light and n² for surface absorption of light, because of angle averaging effects where n is the refractive index of the constituent nanoparticles in the photoactive material.

This light absorption effect is greater for large refractive index values, therefore this effect will be larger for high RI constituents, such as TiO₂ or WO₃ and/or any mixtures thereof.

A perfect back-reflecting layer should provide a factor of 2 enhancement (i.e., from two passes of the light through the photoactive material).

The following materials can be used as a back-reflecting layer: Si-wafers, metallic mirrors (e.g. Ag, Au, Pt, Al), porous Si, mono- and polycrystalline Si, etched Si, Bragg mirrors and reflectors, photonic crystals (e.g., inverse 3D opal structures), for example.

The following materials and texturing techniques can be used for a scattering layer: a layer incorporating large nanoparticles with light-scattering properties (e.g., TiO₂ or ZnO), SiO₂ and polystyrene PS sphere arrays, different surface morphologies and roughness (such as due to etching, calcination, pretreatment processes, and photolithographic treatment), different shape- and form-textured surfaces and/or surface topologies with different shapes, architectures (such as pyramids and cones), gratings and etchings.³⁷

For example, similar to the enhancement of light absorption in Si based photovoltaic (PV) devices³⁸, etching of diffraction gratings or the deposition of a wavelength-specific photonic crystal (such as a Bragg minor or an inverse 3D opal) on the back side (i.e., the side opposite to the incident light) of the photoactive material would help to enhance light absorption peaks associated with constructive Fabry-Perot resonance modes in the photoactive material.

### Gas Permeable and Gas-Barrier Layers

The porosity of the photoactive material is based on the size of its constituent nanoparticles, as well as pore size and/or pore distribution of the constituent layer(s). The selection and manufacture of such characteristics (as described above) allows for control of gas flow, gas diffusion, gas adsorption, gas permeability, gas contact and/or residence time within distinct layers of the material.

In a multi-layered photoactive material, the porosity of different layers can be different. For example, there can be a gradient in porosity ranging from layers with large pore and sparse pore distribution, to layers with small pore sizes and dense pore distribution. Generally, a small pore, also called a micropore, may be about 2 nm in diameter or smaller; a medium pore, also called a mesopore, may be between about 2 to 50 nm in diameter; and a large pore, also called a macropore, may be about 50 nm in diameter or larger. In the disclosed examples, the pores mostly lie in the mesopore range. A sparse pore distribution may result in very few pores in the layer, resulting in an effectively non-porous layer. A dense pore distribution may mean pores cover at least 10% or 50% or more of the surface of the layer.

The photoactive material may also incorporate a gas-barrier layer (shown as 504 in FIG. 5). A gas-barrier layer may allow the photoactive material to be sectioned into separate photoactive portions. Such layers can be made out of very dense films with very small pores that inhibit or prevent the movement of gases through the material. Such gas-barrier layers may allow for separation, fractionation and/or condensation of product and/or reactant gases, for example to prevent produced oxygen gas from reacting with energy-rich fuel products.

### Acid-Base Catalytic Sites

The surface of the nanoparticles in a layer of the photoactive material may include distinct exposed crystal planes, for example with corners and edges that join them. Such exposed metal or semiconductor nanoparticle planes may be similar to theoretical “ideal” lattice planes. Disrupting the crystal network in a metal oxide nanoparticle results in coordinatively unsaturated metal and/or non-metal reaction centers. These unsaturated centers at the surface of the layer allow for gas-solid heterogeneous acid-base catalytic/photoactive reactivity and product selectivity. It is generally known that unsaturated centers at surfaces have higher reactivity, because of lower coordination numbers. Thus, reactivity is increased with increased presence of unsaturated or low-coordination centers on an exposed specific surface. The acidity or basicity of these unsaturated centers results in selective interaction with certain gas-phase molecules, in particular CO₂, H₂ and H₂O, as discussed below.

Basicity and acidity of the constituents affect CO₂ reduction and H₂O or H₂ oxidation, as well as stabilization of separated charge carriers in the constituents. Surface acidity and surface basicity are important characteristics since basicity affects the reaction with CO₂, while acidity affects the oxidation of H₂ and/or H₂O. In general it is always favorable to have a more basic and nucleophilic material (e.g. Cu^{II}O or Cu^{I}₂O) in a low oxidation number (i.e., I or II). A more basic, nucleophilic and electro-rich layer or constituent will bind/activate and react with CO₂; while a more acidic and hole-rich layer or constituent (e.g., Ti^{IV}O₂) will stabilize holes and undergo oxidation with H₂ or of H₂O. This is true for both the single-layer photoactive material as well as the multi-layered photoactive material.

For example, the surface of a solid metal oxide may include one or more of:
Exposed coordinatively unsaturated cationic (metal) centers, which may act as Lewis acid sites
Exposed oxide species, which may act as Lewis base sites
Exposed hydroxy-groups, for example arising from water dissociative adsorption, which may act as Brønsted acid sites, or, alternatively, as basic sites.

Other surface species (e.g., NO, CO or CO₂) can affect the reactivity of the surface, when they have not been decomposed by pre-treatments.

Surface acidity and basicity properties of metal oxide layers can differ in terms of structure and/or composition and the nature of the metal sites involved. The valency, oxidation state and/or atomic size of the metal oxide nanoparticles are factors. Metal oxide materials of different composition may be relevant materials from the point of view of their surface acid-base adsorption and catalytic/photoactive and/or photostoichiometric/photothermal properties. The composition and/or the density of acidic and basic sites on the metal oxide surface are relevant in binding and/or activation of small molecules like H₂O and CO₂.

In some examples, CO₂ activation and adsorption (CO₂⁻)* on metal oxide surfaces may also occur as carbonate (CO₃²⁻), bicarbonate or formate species. CO₂ may be considered a relatively weak Lewis-acid that may interact favorably with relatively strong basic sites due to the electropositive nature of the carbon atom.³⁹ The absorption of CO₂ on any oxide surface may be considered an acid/base reaction e.g. by the addition of a basic oxide ion to acidic CO₂ to form negative carbonate species described according to:

CO₂+O₂⁻→CO₃²⁻

CO₂ adsorption and carbonate formation on the metal oxide surface may occur in most known metal oxides. Infrared analysis of absorbed CO₂ species has shown the formation of different carbonate species, which may occur as monodentate, bridged bidentate or tridentate forms.³⁹ The ability of metal oxides to form carbonates species depend upon their acid/base behavior and the nucleophilic character of the surface oxygen's of the used metal oxide, as explained below. Thus, basic metal oxides in a lower oxidation state (II or I) (e.g. Zn^{II}O, Cu^{II}O or Cu₂^{I}O as well as possible mixed composition thereof) may be favorable for this CO₂ activation-reduction process.

Furthermore, the formation of carbonate species may occur on noble metal surface (e.g. on pure Cu, Ag and Au surfaces) with an activated and atomically adsorbed oxygen atom at the surface.

The following table^{4a} provides a summary of acid-base properties of example binary metal oxides:

| Oxidation | Acidity | Acidity | Basicity, | Metal Oxide |
|---|---|---|---|---|
| state | type | strength | nucleophility | Examples |
| >+5 | Brønsted | Medium | None | P₂O₅ |
| | | strong | | |
| +3 to +4 | Brønsted | Medium | None | SiO₂, GeO₂ |
| | | weak | | |
| +5 to +6 | Brønsted | Medium to | None | WO₃, Ta₂O₅ |
| (high) | & LA | strong | | |
| +3 (medium) | LA (Lewis | Strong | Weak | γ-Al₂O₃, |
| | Acid) | | | β-Ga₂O₃ |
| +3 to +4 | LA (Lewis | Medium | Medium | TiO₂, Fe₂O₃ |
| | Acid) | | weak | |
| +4 | LA (Lewis | Medium | Medium | SnO₂, CeO₂ |
| | Acid) | weak | strong | |
| +1 to +2 | LA (Lewis | Medium | Strong | MgO, CoO, CuO, |
| (low) | Acid) | to very | to very | ZnO, NiO, Cu₂O |
| | | weak | strong | |

Non-photoactive materials, for example γ-Al₂O₃ or MgO, may also be used as acid or basic supports, for example when mixed together with photoactive layers or catalytic photoadditives and/or promoters. In some example embodiments, based on the formation of mixed low refractive metal oxide thin films, and their acid-basic properties, such an example composition may lead to a higher CO₂ absorption and may result in a more efficient photochemical reduction. Al₂O₃ in this example may act as an adsorbing and activating support layer.

In general a generated electron rich layer may be favorably positioned or generated on a more basic material, and the generated hole rich layer may be favorably positioned or generated in a more acidic material.

### Hole Scavengers and Electron Trapping Materials

In order to enhance charge carrier separation in electron- and hole-rich layers, “hole scavenger” and “electron trapping” materials may be incorporated. Hole scavengers tend to attract holes while electron trapping materials tend to attract electrons. In the hole-rich layer (e.g., a layer including a p-type semiconductor constituent), metal oxides, which may be considered a hole scavenger may be incorporated or generated in-situ through, for example, thermal or photochemical reduction or by salt impregnation techniques.

Generally, a hole scavenger is defined as a semiconductor material in which electrical conduction is due chiefly to the movement of positive holes. An example of a hole scavenger is a p-type semiconductor material. Similarly, an electron trapping material is defined as a semiconductor in which electrical conductivity is due chiefly to the movement of electrons. An example of an electron trapping material is an n-type semiconductor material.

Such hole scavengers include, for example, RuO₂, IrO₂, NiO, Co₃O₄, Ni(BO₂)₂×H₂O, RuO₂, IrO₂ and Co(BO₂)₂Co⁴⁰. In the electron-rich layer, what may be considered “electron trapping” materials, such as noble metal nanoparticles may be incorporated or generated in-situ through, for example, thermal and/or photoreduction processes. Such electron trapping materials include, for example, Pt, Cu, Ag, Au, Cu, Fe₃C, SiC or C-dots¹³. Such electron tapping materials also include basic and nucleophilic metal oxides, for example ZnO, CuO, Cu₂O and mixtures thereof.

### Examples of Redox Behavior of Photoactive Metal Oxide Layers

In some examples, oxidizing photocatalysts (e.g., of V₂O₅, MnO₂, InTaO₄ or BiVO₄) may be involved in mild or total oxidation processes of hydrocarbons or of other molecules (e.g. to selective alcohol formation of MeOH or EtOH). For the oxidation step, the surface lattice oxygen (O²⁻) of the employed metallic oxides may play a role in the selective formation of the desired product. This phenomenon may be generally known as redox catalysis, which may occur in a two-step reaction scheme below, describing this participation:

Cat-O+Red→Cat+Red-O and

Cat+Ox-O→Cat-O+Ox

In this example, the exposed oxide catalyst surface (Cat-O) may get reduced by a reductant (Red, e.g. an organic compound) reoxidized back through an oxidant (Ox-O, e.g. formed O₂) to its initial stage.^{4a}

For example, the properties of (O₂⁻) species linked to metallic cations may determine the catalytic/photoactive properties, for example affecting the selectivity of the reaction products. A possible consideration may be the formed nucleophilic (O₂⁻) and electrophilic (O₂⁻, O₂²⁻) oxygen species, which may play a role in mild and total oxidations.

The presence of extra oxidizing photocatalysts helps to increase the selectivity of an oxygen-rich compound (e.g. TiO₂/Fe₂O₃) in production of CH₄. Further oxidization of CH₄ to CH₃OH, which is a redox two-step reaction, is aided by the presence of additional oxidizing photocatalysts (e.g., MnO₂ or BiVO₄) which may increase the amount of oxygen-rich fuel product, thereby shifting the photoreaction selectivity from production of CH₄ to production of CH₃OH.

### Additives

Various functional components can be incorporated into the disclosed photoactive materials. These additives can help to enhance the redox reactions carried out in the photoactive materials by boosting the reaction rate and/or selectivity. Such additives may be incorporated during manufacture of the nanoparticle layers, for example by introducing the additives into the colloidal suspension during manufacturing.

Possible additives include co-catalysts, promoters, plasmonic converters, up-converters and down-converters. While incorporation of such additives into a layer arrangement is generally straightforward, this may be difficult or impossible for conventional photonic crystals having 2D or 3D periodicities. For example, photonic crystals having 2D or 3D periodicities typically are more difficult to manufacture (e.g., requiring a specific template), require depositing of any additives through several treatments, and result in films of typically lower optical quality.

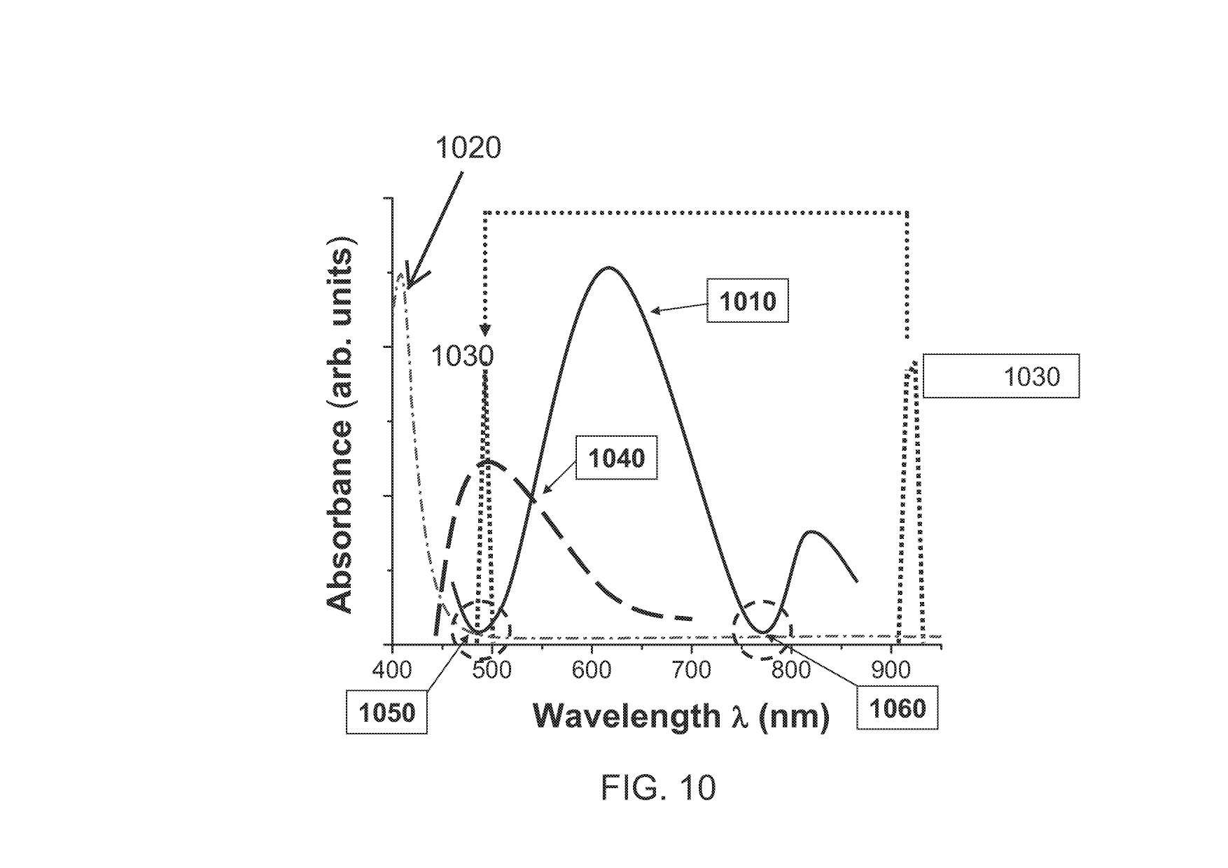

FIG. 10 is an example absorbance spectrum schematically illustrating how the incorporation of plasmons, up-converters and slow photon effects may contribute to the optical absorbance 1020 of metal oxide nanoparticles. The optical absorbance spectrum 1020 exhibits a photonic stop band 1010. The addition of up-converters results in conversion of absorbance at high wavelengths 1030 to absorbance at lower wavelengths UC. The addition of plasmonic additives results in surface plasmon resonance effects 1040. Slow photon effects result in enhanced absorption at the blue edge 1050 and red edge 1060 of the photonic stop band 1010. These effects are described in greater detail below.

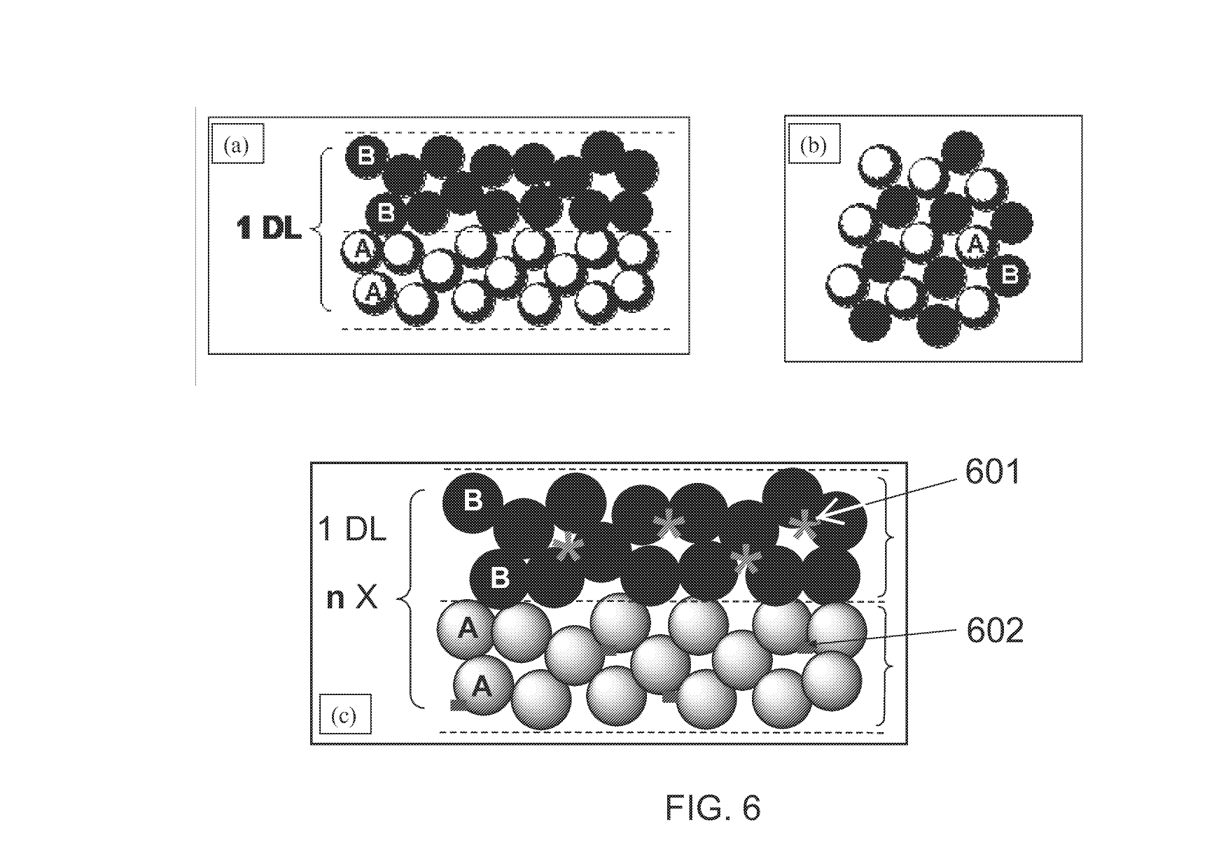

FIG. 6C illustrates a multi-layered photoactive material, formed as a bilayer of photoactive constituents A and B incorporating plasmonic additives 601 (such as Au, Ag and/or Cu) in one layer and up- and/or down-converters 602 in another layer. It should be understood that other additives, including those discussed below, may be incorporated into the photoactive material. Although this example shows different additives being incorporated into different layers, it should be understood that one or more additive may be common among the layers, and that one or more layers may have no additives. Although this example shows a multi-layered photoactive material, it should be understood that one or more additives may be similarly incorporated into a single-layer photoactive material.

Examples of Co-Catalysts, Catalytic Additives & Noble Metal loaded Metal Oxides

Incorporation of noble metals and/or catalytic additives (such as different co-catalysts and/or promoters) into the photoactive material may help to enhance the photoactivity of the material. Examples of such additives include Pt, Au, Ag and Cu. An incorporated noble metal and/or co-catalyst will act as a sink for generated charge carriers (i.e., electrons and holes), thereby reducing the rate of electron-hole recombination. Incorporated noble metal nanoparticles will help to absorb more light and may help to enhance the lifetimes of the excited electrons and holes.

The following examples of transition and noble metal nanoparticles and compositions, co-catalysts and alkali/earth-alkali based promoters may be added/incorporated in the photoactive material:

### Examples of Co-Catalytic Additives:

ZnO, NiO, TiO₂, ZnSe, CdS, GaP, GaN, MnO₂, Fe₂O₃, CdSe, CuO, Cu₂O, PtO, CoO, PdO, Co₃O₄, Rh₂O₃, RuO₂, IrO₂, Ag₂O, Au₂O₃, SiC, Fe₃C, WC SnO₂, ITO≡In₂O₅:Sn (Indium Tin Oxide), ATO≡SnO₂:Sb (Antimony Tin Oxide), FTO≡SnO₂:F (Flourine Tin Oxide), ZTO≡SnO₂:Zn (Zinc Tin Oxide), IZO≡In₂O₅:Zn (Indium Zinc Oxide), and similar species

Examples of Transition and Noble Metals Nanoparticle Compositions:

C, Ti, Zr, Hf, V, Nb, Ta, Cr, Mo, W, Mn, (Tc), Re, Fe, Ru, Os, Co, Rh, Ir, Ni, Pd, Pt, Cu, Ag, Au, Zn, Cd, (Hg)

In some examples, different alloyed nanoparticles, multimetal (M₁/M₂) and multimetal oxide M^{a}₁₋ₘM^{b}ₐM^{c}_{b}M^{d}_{c}MⁿₘO_{y} as well core-shell structures denoted as M₁@M₂ (M₁ and M₂) and/or heterodimeric nanoparticle assemblies M₁O-M₂O (M₁ and M₂) nanoparticles may be incorporated as co-catalysts.

For example, the following catalytic alkali and/or earth-alkali promoters may be incorporated, for example as impregnated or deposited salts on the surface of a layer of the photoactive material:

K₂O, Na₂O, Li2O, BeO, MgO, CaO, CsO, SrO, BaO, NaOH, KOH, LiOH, Ca(OH)₂, Mg(OH)₂, Sr(OH)₂, Ba(OH)², NaHCO₃, Na₂CO₃, K₂CO₃, Li₂CO₃, NaCl, Na₂SO₄, Na₃PO₄, Na₂HPO₄, and various mixtures thereof.

### Plasmonic Additives

The incorporation of plasmonic additives, such as noble metal nanoparticles, in the photoactive material can also help to enhance optical absorption by inducing SPR⁴¹ of the photoactive constituent nanoparticles. SPR originating in conduction electron oscillations in metal nanoparticles smaller than the wavelength of light is useful for their ability to confine and intensify light in small volumes. SPR amplifies incident light at certain wavelength ranges, described in the literature⁴¹, which results in amplification of the photoactivity of the photoactive material.

Selection of a plasmonic additive can be based on their known absorption wavelength ranges. For example, spherical Au nanoparticles may be selected to amplify absorption in the range of about 450 to 650 nm, with a peak maximum at around 525 nm²³; spherical Ag nanoparticles may be selected to amplify absorption in the range of about 350 to 500 nm, with a peak maximum at around 410 nm; and spherical Cu nanoparticles may be selected to amplify absorption in the range of about 520 to 650 nm, with a peak maximum at around 570 nm.

The specific SPR absorption-band of the incorporated noble metal constituent(s) can be selected to lie at a desired wavelength range, for example in the visible and/or near infrared wavelength region (e.g., 450-1500 nm). Using the SPR of incorporated plasmonic additives, more efficient charge carrier generation and separation processes may occur for electrons and holes generated to be used in photoactive reactions for a given amount of incident light.

Examples of such plasmonic additives include metals (e.g., Ag, Au and Cu), alloys and core-shell structures M¹@M²Oₓ (e.g., Cu@CuO, CuO@Cu, Au@Fe₂O₃ or Cu@SiC compositions), as well as various plasmonic heterodimeric nanoparticle assemblies M₁O-M-M₂O (e.g., NiO—Au—CuO, WO₃—Ag—Fe₂O₃, Fe₂O₄—Au—CuO and ZnO—Cu—Fe₂O₃).

The incorporated SPR modes of metallic nanoparticles may be tightly confined to the adjacent photoactive nanoparticle, for example with skin depths of the order of tens of nanometers.

The effectiveness of plasmonically enhanced photoactivity depends on the tuning of the SPR band of the incorporated plasmonic additive into the electronic absorption wavelength region of the photoactive layer. Such tuning of the SPR may be achieved by selecting the plasmonic additive to be incorporated into the photoactive material.

One approach is the design and implementation of alloyed particles M1/M2 (e.g., Au/Ag or Au/Pt). Another approach is to make different core-shell structures, generally M₁-M₂ where M1=Ag or Au; M2=Au, Pt, Pd, Rh, Ir, Ru, Cu, Os, Cr, Mn and similar species. The use and incorporation of a trimetallic (e.g., Ag—Au—Pt) or multimetallic core-shell system, generally M₁@MOₓ, nanoparticles can also be useful for obtaining desired optical and/or catalytic features, as discussed above.

Plasmonic additives can be incorporated at various locations within the photoactive material including: embedded within the layer(s), embedded at the interface of the layers in a multi-layered photoactive material, or deposited/embedded on the top or final layer of the photoactive material.

Plasmonic amplification effects can also coupled with slow photon enhancement effects, as described above, at a specific energy or wavelength range. The plasmonic additives may provide a local enhancement induced by the localized surface plasmons. The specific energy- and wavelength-dependent absorption of localized surface plasmons may be increased by slow photon effects in the same or overlapping energy region. The result of this synergism is a local SPR field enhancement and enhanced plasmonic absorbance in the photoactive material.

By combining plasmonic and slow photon amplification effects, the excitation of generated electron-hole pairs may be increased, which may help to increase the rate of a gas-solid photoactive reactions.

### Up-Converters

Up-converter nanoparticles may be selected to convert incident light from one wavelength to a second wavelength, for example converting incident near infrared (NIR) wavelength light to visible wavelength ranges. NIR to visible wavelength up-converter nanoparticles incorporated into the photoactive material can help to harness NIR light for photoactive reactions.

Examples of such up-converters include: rare earth doped or co-doped host compounds, such as NaYF₄, LaF₃, La₂(MoO₄)₃, among others known in the art.

Combining up-converter nanoparticles with plasmonic nanoparticles in the photoactive material may result in improved photoactivity in response to light ranging from NIR to the visible to the UV range.

### Example Study

An example study of the photoactive material is now described. This example is for the purpose of illustration and is not intended to be limiting.

### Preparation of the Photoactive Material

In this example, a 1×1 inch (about 2.5×2.5 cm) photoactive material was tested, in which the photoactive constituent nanoparticles were Fe₂O₃/TiO₂. The material was manufactured on a substrate, in this case fluorine-doped tin oxide (FTO)-coated glass, SiO₂-coated glass and Si-wafer, which are commercially available. These substrates were pretreated and cleaned before spin-coating.

Prior to spin-coating, the substrate was treated with a mixture of H₂O₂/H₂SO₄ (3:1) and H₂O₂/NH₃H₂O (3:1) for at least 1 hour and washed after the treatment with ethanol. The Si wafers and FTO-coated glass were further treated under air plasma for at least 5 min to remove impurities and to increase the hydrophilicity of the surface.

The Fe₂O₃ nanoparticles were synthesized by dissolving Fe(NO₃)₃9H₂O (5.05 g, 12.5 mmol) in 80 mL ROH, with R=Me, Et, n-Pr, iso-Pr, or tert-Bu, followed by addition of 20 mL deionized water (0.056 AS/cm). The resulting dark-red solution (pH≈1-2) was stirred for 12 h at room temperature (RT). The resulting orange-brown Fe₂O₃ dispersion was stored at RT in air.

Another method of synthesizing Fe₂O₃ nanoparticles was by simple dissolution of 3 g of the elemental Fe metal powder (mesh 100 or 325), dispersed in 10-15 ml of deionised H₂O (0.056 μS/cm) followed by the addition of 10-35 mL H₂O₂ (30%. p.a.) and 3 mL of AcH (Glacial Acid) (ratio≈10:1) at 0° C. in an ice-bath under air and further stirring for 3 day under RT, no inert atmosphere (nitrogen) needed. Since this is a very exothermic reaction, instant ice-bath cooling is necessary in a well ventilated hood.

TiO₂ (rutile form) nanoparticles were synthesized using 18.75 mL of Ti(OiPr)₄ Titanium-iso-propoxide added dropwise under vigorous stirring at RT (Room Temperature) to 110 mL of an aqueous 0.1 M nitric acid (HNO₃) mixture. The resulting slurry was heated at 80-90° C. for an additional 8 hours, the resulting white-milky TiO₂ dispersion was cooled down to RT and the dispersion was stored at RT in a brown glass vessels for further use.

In another method of synthesis, TiO₂ (anatase form) nanoparticles were synthesized using 17 mL of Ti(OiPr)₄ Titanium-iso-propoxide added dropwise under vigorous stirring at RT (Room Temperature) to 80 mL of MeOH. After addition of 2 mL of AcH (Glacial Acid) and ≈1-2 ml of distilled water the resulting slurry was heated at 80-90° C. for an additional 8 hours, the resulting white-milky TiO₂ dispersion was cooled down to RT and the dispersion was stored at RT in a brown glass vessels for further use.

In another method of synthesis Sb:TiO₂ (anatase form) nanoparticles were synthesized using 17 mL of Ti(OiPr)₄ Titanium-iso-propoxide added dropwise under vigorous stirring at RT (Room Temperature) to a mixture of 80 mL of MeOH with dissolved Sb(OAc)₃ 30-50 mg (0.1 to 0.170 mmol). After further addition of 2 mL of AcH (Glacial Acid) and ≈1-2 ml of distilled water the resulting slurry was heated at 80-90° C. for an additional 8 hours, the resulting yellow-milky TiO₂ dispersion was cooled down to RT and the dispersion was stored at RT in a brown glass vessels for further use.

The prepared metal oxide dispersions were filtered through a 0.45 μm Titan 2 HPLC Filter Amber (GMF Membrane), to remove any agglomerates and subsequently diluted to the desired concentration, used for porous layered photoactive materials. The dispersions were diluted with deionized water to the desired concentration (ranging from 3 wt. % to 35 wt. %) and Polyethylene glycol (PEG, [(C₂H₄O)nH₂O], MW: 20.000 g/mol) was added and dissolved in the range of 1-20 wt % to prepare spinable dispersion forms before spin-coating.

To evaporate the dispersion solvent from the dispersion, spin-coating of the nanoparticle layer was performed on a Lauriel single wafer spin processor (Model WS-400A-6NPP/LITE) at 2500-6000 rpm, 25-60 acceleration for 20-60 sec. The resulting porous nanoparticle metal oxides thin layers were calcined at 450-600° C. for 15-60 minutes.

To prepare a multi-layered photoactive material, a pair of two different nanoparticle layers were spin-coated from modified PEG-dispersions and subsequent calcined, iteratively until the desired number of layers was deposited using various nanoparticle dispersions.

Although the above example describes certain manufacture conditions, these may be varied. For example, spinning conditions may be varied, for example as follows: spin-coating time about 5 sec to 5 mins, about 5-6000 rpm with different acceleration conditions.

Calcinations may be varied by different temperatures (e.g., about 5 to 2000° C.) and through different calcination times (e.g., about 5 min to 1000 h), and different post-treatment procedures (e.g., oxidation/reduction processes) may be included.

These metal oxide nanoparticles may be produced, for example, by a variety of known synthesis methods and variations. Such methods include, for example, sol-gel processes, basic precipitation syntheses, deposition precipitation processes, hydrothermal processes, ceramic processes, reduction/oxidation processes of dissolved metal salt precursors, and colloidal electrochemical processes, among others.

Porous thin films may be produced from different sources, such as from commercial and/or self made dispersion, powders, and/or solid materials/targets. Such films may be made by various deposition technique, including, for example: spin-coating, dip-coating, spray pyrolysis (SP), ultrasonic spray pyrolysis (USP), spray-coating, aerosol-coating, drop-casting, doctor-blading, draw-bar, screen-printing, and ink-jet-printing, reactive DC magnetron sputtering, atmospheric pressure chemical vapour deposition (APCVD), metal oxide chemical vapour deposition (MOCVD), molecular beam epitaxy (MBE), pulsed laser deposition (PLD), oblique angle deposition (OAD), glancing angle deposition (GLAD), potentiostatic anodization and electrodeposition. The manufacturing may include two or more deposition techniques, e.g. sol gel spin coating and sputtering or CVD techniques. Any other suitable methods may be used. Two or more techniques, including those described above, may be used together.

### Photo-Sabatier Process on Fe₂O₃/TiO₂ Photoactive Material

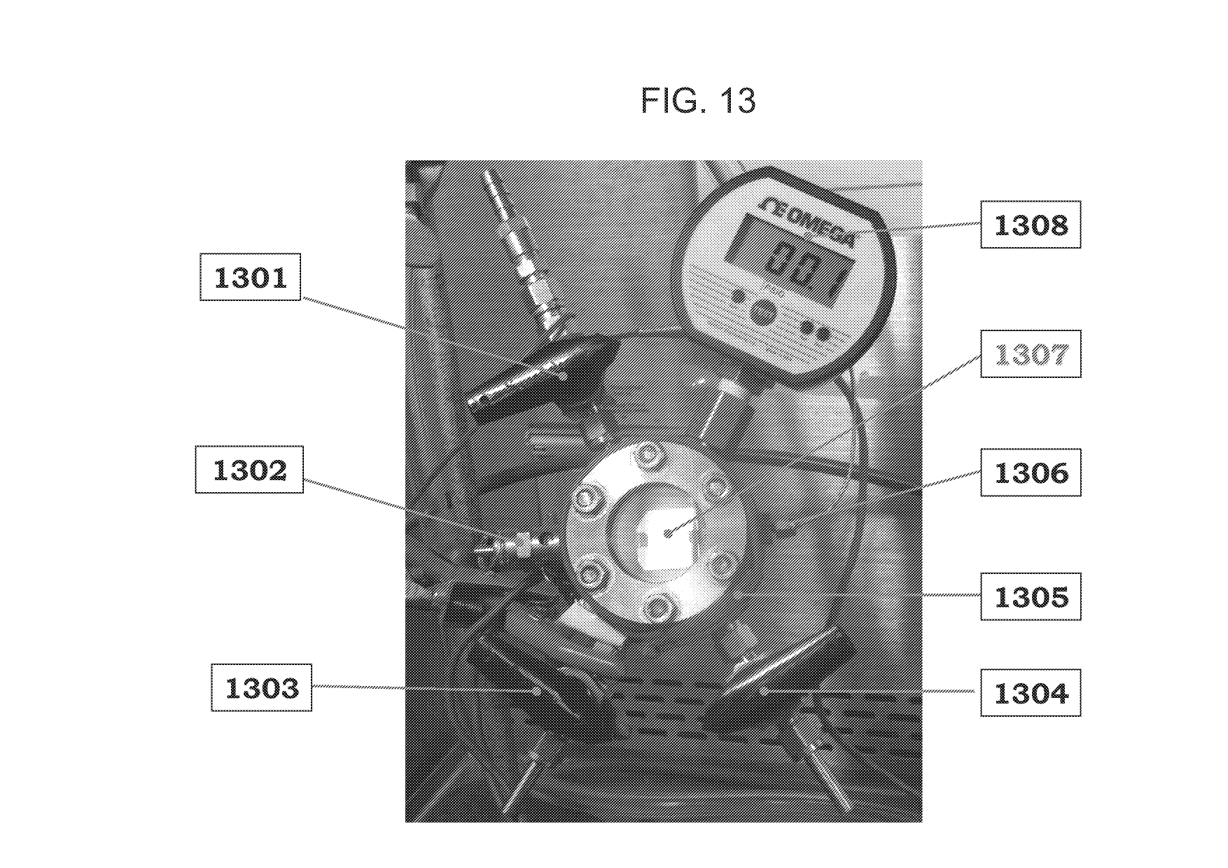

The Photo-Sabatier process, namely CH₄+4H₂→CH₄+2H₂O, was examined by comparing conversions in the dark, pure UV light and an air mass (AM) 1.5 sunlight-filter at different reaction temperatures (ranging from 40° C. to 85° C.). This was tested using the photoreactor shown in FIG. 13.

FIG. 13 shows a batch test photoreactor having a total reaction volume of 28 mL. The photoreaction was equipped with two gas (specifically CO₂ inlet valve 1303 and H₂/H₂O inlet valve 1304) inlet valves as well as one gas outlet or vacuum valve 1301. The batch test photoreactor also included a thermocouple 1306 which measured the temperature inside the gas reaction, a safety valve 1302 (max. 100 psi) and a 1×1 inch holder for holding the sample photoactive material 1307. For heating the chamber to reaction temperatures of 40 or 80° C., a heating mantel 1305 was wrapped around the chamber. A digital pressure gauge (DPG) 1308 was used for real-time monitoring and recording of the actual pressure data and relative pressure change during the 18 h reaction time period.

The photoactive material was placed inside the photoreactor, the reactor was evacuated, tightened and sealed with screws. Then CO₂ gas (99.995% purity) and H₂ or a (H₂/Ar 99.995%) 50:50 gas mixture were pressurized (to a maximum of 100 psi) in a 1:4 ratio inside the pilot-batch reactor. Photolytic CO₂ reduction was carried out, by using different reaction temperatures (ranging from 40° C. to 85° C.) with a 200 W high-pressure HgXe lamp over a period of 18 h. To simulate sunlight irradiation, the 1.5 AM sunlight filter was used. On-line monitoring of pressure and temperature changes during the reaction was done by a digital pressure gauge and a thermo-couple installed inside the reactor chamber.

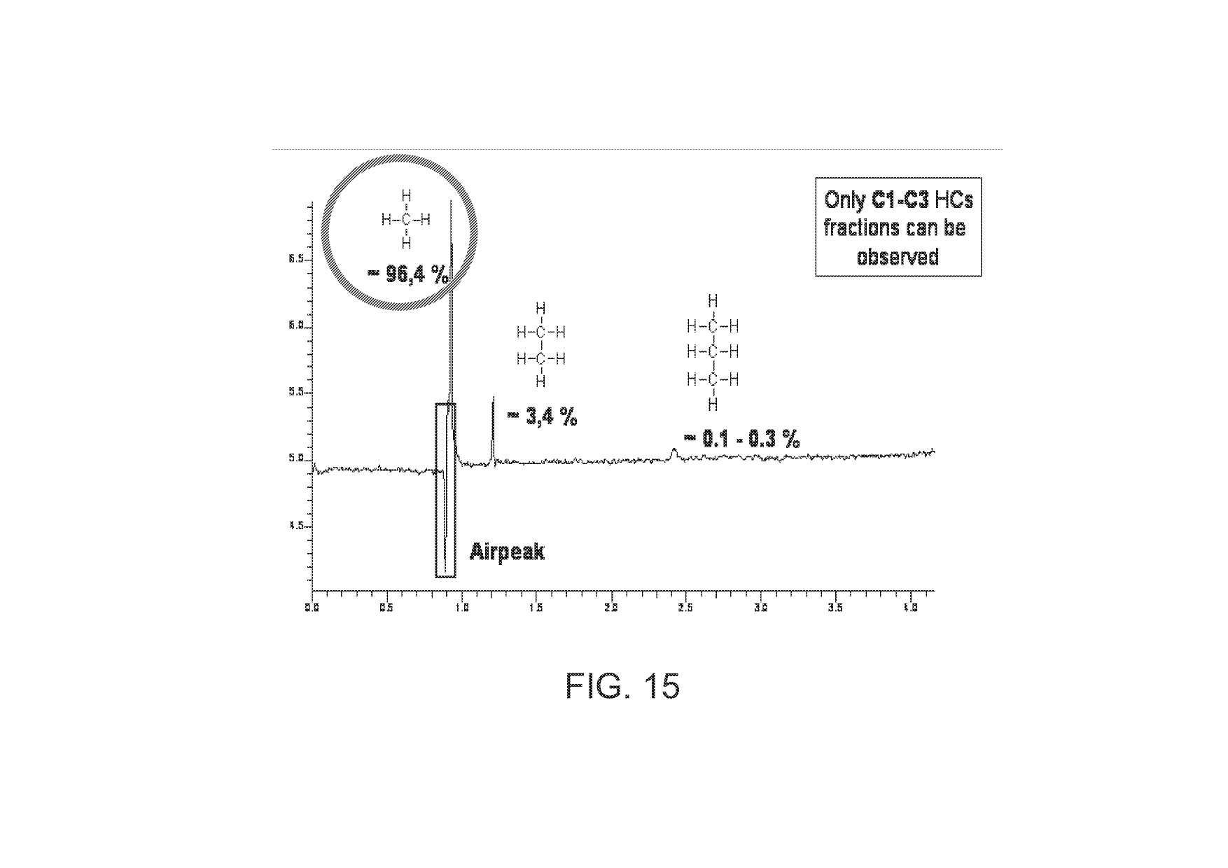

The photoreactor was operated in batch mode with temperature control, pressure monitoring and subsequent batch analysis after 18 h by gas chromatography (GC) by using a Perkin Elmer (PE) Auto System XL GC with a flame ionization detector (FID) on a GS-GASPRO column (measuring 30 m×0.32 mm). An example of the gas-phase batch GC measurements is shown in FIG. 15. As shown in this example, only fuel products having low weights (e.g., C₁-C₃) could be monitored, with C₁ products, namely methane, dominating. The relative rate of conversion of carbon dioxide (CO₂) to methane (CH₄) was approximated from the change in hydrogen partial pressure as a function of time and was subtracted from previous recorded blank and/or reference runs, determined from reaction stoichiometry, in the batch photoreactor over an 18 h period.

### Results

The external quantum yield (EQY) for the conversion of carbon dioxide into methane by the photoactive film in the photoreactor was evaluated by using a fiber optic coupled integrating sphere and a calibrated spectro-radiometer (from Stellarnet) to measure the total number of photons hitting the samples (with a powder density of ≈100 W/m²) per unit time and relating this to the relative and average rate number of moles of methane (with conversion rates ranging from μmol·g⁻¹·h⁻¹ to mmol·g⁻¹·h⁻¹ based on the catalyst weight, as well as average rates in μmol·m⁻²·s⁻¹ based on the catalyst surface area produced per m² per unit time).

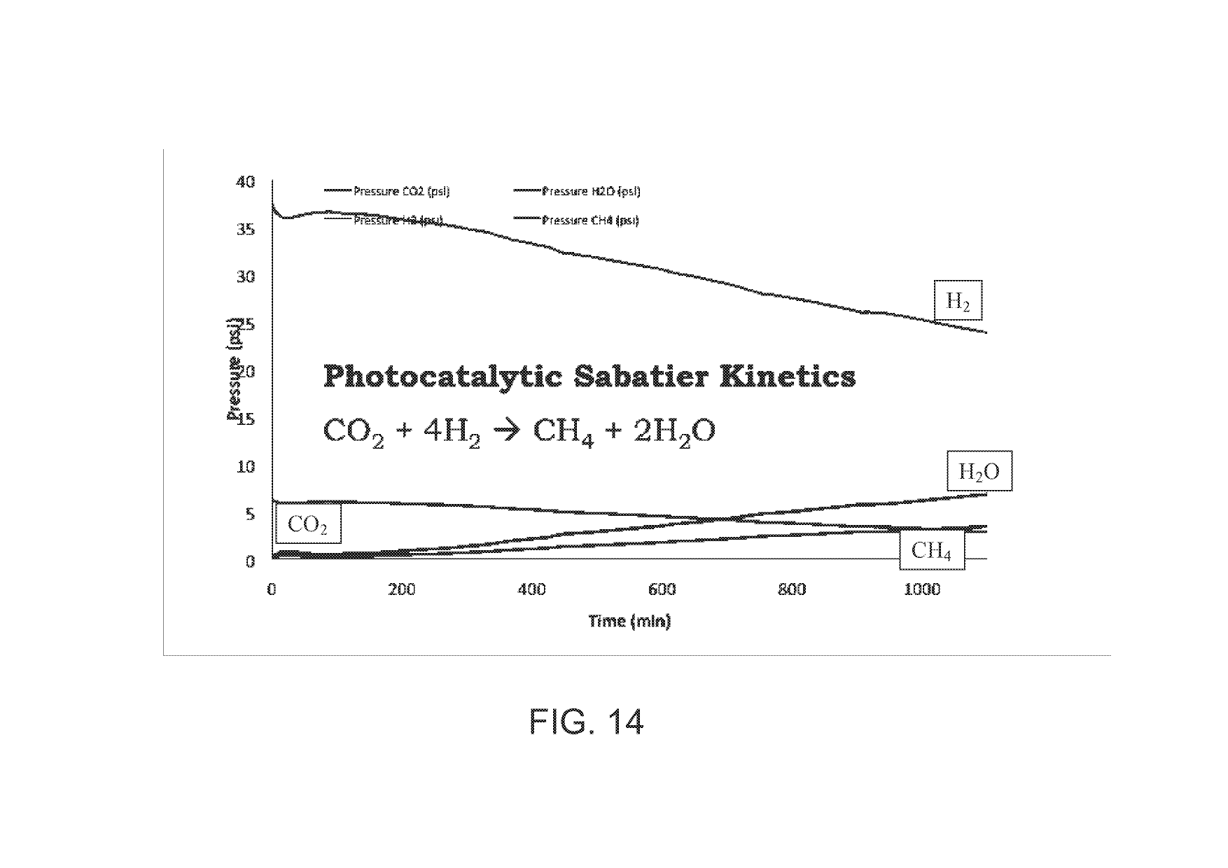

The results are summarized in the table below and in FIG. 14. FIG. 14 shows the monitored and calculated pressure changes of gaseous reactants CO₂ and H₂, and gaseous products CH₄ and H₂O for the AUltra. 8 DL sample at 80° C. AM1.5.

| | | | | | rate | |
|---|---|---|---|---|---|---|
| | React. | max. PBG, | Weight | rate (average) | (average) | EQY (Φ) |
| Composition | Conditions | amount DL | (mg) | mmol · g⁻¹ · h⁻¹ | μmol · m⁻² · s⁻¹ | 350-600 nm |
| Fe₂O₃/TiO₂ | 40° C., UV | NIR, 4 DL | 2.6 mg | 0.67 | 0.77 | 4.31 |
| Fe₂O₃/TiO₂ | 80° C., UV | NIR, 4 DL | 2.6 mg | 0.84 | 0.97 | 5.43 |
| Fe₂O₃/TiO₂ | 80° C., AM1.5 | NIR, 4 DL | 2.6 mg | 2.07 | 2.4 | 25.52 |
| Fe₂O₃/TiO₂ | 40° C., UV | Yellow, 5 DL | 1.7 mg | 3.7 | 2.8 | 15.68 |
| Fe₂O₃/TiO₂ | 80° C., UV | Yellow, 5 DL | 1.7 mg | 2.77 | 2.1 | 11.76 |
| Fe₂O₃/TiO₂ | 80° C., AM1.5 | Yellow, 5 DL | 1.7 mg | 4.92 | 3.72 | 39.55 |
| Fe₂O₃/TiO₂ | 40° C., UV | Green, 6 DL | 1.5 mg | 1.8 | 1.2 | 6.72 |
| Fe₂O₃/TiO₂ | 80° C., UV | Green, 6 DL | 1.5 mg | 1.83 | 1.22 | 6.83 |
| Fe₂O₃/TiO₂ | 80° C., AM1.5 | Green, 6 DL | 1.5 mg | 6.61 | 4.41 | 46.89 |
| Fe₂O₃/TiO₂ | 40° C., UV | SUltra, 8DL | 1.4 mg | 3 | 1.9 | 10.64 |
| Fe₂O₃/TiO₂ | 80° C., UV | SUltra, 8DL | 1.4 mg | 2.85 | 1.77 | 9.91 |
| Fe₂O₃/TiO₂ | 80° C., AM1.5 | SUltra, 8DL | 1.4 mg | 6.73 | 4.18 | 44.44 |
| Fe₂O₃/TiO₂ | 40° C., UV | AUltra, 8DL | 1.0 mg | 3 | 1.9 | 10.64 |
| Fe₂O₃/TiO₂ | 80° C., UV | AUltra, 8DL | 1.0 mg | 0.54 | 0.24 | 1.34 |
| Fe₂O₃/TiO₂ | 80° C., AM1.5 | AUltra, 8DL | 1.0 mg | 8.7 | 3.86 | 41.04 |
| Fe₂O₃/TiO₂ | 40° C., UV | NUltra, 8DL | 0.9 mg | 4.7 | 1.9 | 10.64 |
| Fe₂O₃/TiO₂ | 80° C., UV | NUltra, 8DL | 0.9 mg | 5.2 | 3.2 | 17.93 |
| Fe₂O₃/TiO₂ | 80° C., AM1.5 | NUltra, 8DL | 0.9 mg | 5.7 | 4.2 | 44.65 |
| Fe₂O₃—TiO₂ | 40° C., UV | Mixed Film | 1.5 mg | 1.4 | 0.94 | 5.27 |
| Fe₂O₃—TiO₂ | 80° C., UV | Mixed Film | 1.5 mg | 0.49 | 0.33 | 1.85 |
| Fe₂O₃—TiO₂ | 80° C., AM1.5 | Mixed Film | 1.5 mg | 5.51 | 3.67 | 7.12 |

Table abbreviations:
PBG = photonic band gap;
DL = double layer;
EQY = external quantum yield;
UV = ultraviolet;
NIR = near infrared;
AM 1.5 = air mass coefficient/simulated sunlight;
SUltra = ultra-thin layers prepared by solvent;
AUltra = ultra-thin layers prepared in MeOH/acetic acid;
NUltra = ultra-thin layers prepared in water/nitric acid.
Ultra-thin layers had thicknesses in the range of about 25-40 nm.

The above table contains results of the sample Fe₂O₃/TiO₂ photoactive materials. The multi-layered arrangements have 4, 5, 6 and 8 DL. The layer thicknesses ranged from very thick (e.g. 4 DL of 180 nm thick Fe₂O₃ and 160 nm thick TiO₂ NIR samples) to ultra thin (e.g. 8 DL of 25 nm thick Fe₂O₃ and 30 nm thick TiO₂ AUltra samples). The examples also included single-layer photoactive materials.

It was found that photoactivity increases with decreasing layer thickness even when fewer amounts of the photoactive constituents are used. For example, 1 mg of constituents for the AUltra samples have higher photoactivity (up to about 4-5 times), comparable to 2.6 mg of constituents for the NIR 4DL layers. Aside from the benefits of thinner and ultra-thin (about 20-25 nm thick) layer thicknesses, an increase in contact surface area between adjacent ultra thin layer constituents may also contribute to this higher photoactivity. As a general trend, the multi-layered architecture, especially those having ultra thin layers, showed improved photoactivity compared to mixed single-layer architectures. Also such mixed single-layers, which can be described as having closely packed mixed constituent, show lower photoactivity, their simpler architecture and manufacturing may facilitate their use in large-scale applications.

Just comparing the ultra-thin samples (i.e., SUltra, AUltra and NUltra), reaction rates can be seen to be dependent on the TiO₂ particle sizes. For example, the SUltra (Sol-TiO₂) layers are quite dense with large particle sizes in the range of ≈20-25 nm; the AUltra (acetic acid, anatase form TiO₂) layers have particle sizes in the range of ≈12-15 nm; and the NUltra (nitric acid, rutile form TiO₂) layers have very small particle sizes, in the range of about 4-6 nm. Experimental results showed that, rather than the ultra-small TiO₂-rutile nanoparticles having the highest surface area having the best reactivity, it was rather the TiO₂-anatase preparation with particle sizes of about ≈12-15 nm that showed the best performance. This is likely due to the porosity-surface area trade-off, and less surface defects, discussed above.

In all the above samples, the Fe₂O₃ layer had the same porosity with particle sizes of about 4-7 nm. The Fe₂O₃ layer was prepared in ethanol/H₂O, with layer thickness ranging from 180 nm in the MR samples to about 25 nm in the ultra-thin samples. Specific porosity for Fe₂O₃ layers was about 0.223 cc/g or 42% relative humidity, as measured by EP.

The results show testing on variants of the photoactive material, including non-photonic crystal multi-layered arrangements, mixed single-layer arrangements, and photonic crystal multi-layered arrangements. It was found from GC analysis of the gas after 18 hour that the reactants carbon dioxide and hydrogen react to selectively form methane and water. The selectivity to methane is around 96% the other 4% being ethane and propane, similar to the composition of natural gas (see FIG. 15).

The results of this study indicate that a combination of Fe₂O₃ and TiO₂ photoactive nanoparticles is capable of activating the Photo-Sabatier Process CH₄+4H₂→CH₄+2H₂O at 40° C. and 80° C., producing methane (CH₄) at ≈0.67 mmol·g⁻¹·h⁻¹ up to a maximum of 8.7 mmol·g⁻¹·h⁻¹. The EQY in the absorption range of the selected material, in the range of 350 to 600 nm, was up to 47%. A photoactive material, to be suitable for economical use on a large scale, preferably should display a quantum efficiency of greater than 10% in the visible region of the solar spectrum.

In this study, the rates of CO₂ uptake under low light irradiance (i.e., 200 to 400 μmol photons m⁻²·s⁻¹) were comparable to average plants in the natural world (around 6-8 μmol m⁻² s⁻¹)⁴².

The effect of layer thickness was observable, with average relative rates of production increasing with decreasing of the layer thickness. This can be seen in the ultra-thin layer configurations, such as Fe₂O₃≈25-30 nm and TiO₂≈30-40 nm. Higher light irradiance flux with higher power density (up to ≈5000 W/m²), such as available with concentrated solar power (CSP) may yield higher conversion and quantum efficiency numbers.

Although this study was carried out using a batch reactor, the Photo-Sabatier reaction can be also carried out in a flow-through reactor.

Based on the results of this study, at a relative average conversion rate of carbon dioxide to methane of about 8.7 mmol·g⁻¹·h⁻¹; or ≈5 μmol m⁻² s⁻¹, about one billion 1 m² solar panels incorporating the Fe₂O₃/TiO₂ photoactive material of this example, spread over an area of about 1000 km² should be sufficient to recycle 10¹⁰ tons per year of carbon dioxide currently emitted into the earth's atmosphere.

### Applications

### Photoreactors

Industrial implementation of photoreactions may be done through the use of a photoreactor. A photoreactor is typically a device configured to bring photons and reactants into contact with a photoactive material and is typically also configured to collect the reaction products. Photoreactors may differ from other chemical reactors in that the physical geometry of the photoreactor may be configured to help ensure that photons are concentrated and/or collected effectively.

The disclosed photoactive materials may be suitable to be incorporated into photoreactors⁴³ for photon-driven generation of fuels, in particular hydrocarbons and oxygen-rich hydrocarbon compounds, from carbon dioxide. To enable this application, the photoactive materials may be manufactured as optically transparent solar panels, membranes or coatings.

As described above, the photoactive material can be designed to have high intrinsic optical and photoactive quantum yields as well the ability to select for reactivity to certain wavelengths of light (also referred to as “color tunability”).

The disclosed photoactive materials may be incorporated into solar panels, membranes and/or coatings and be connected, coupled, deposited and/or coated to a large scale hydrogen source energy system and/or solar thermal hydrogen production unit. Examples of systems and devices that may incorporate the disclosed photoactive materials include photoelectrochemical cells (PECs), small and large scale industrial reforming processes, off-shore and on-shore coal, oil and gas reservoirs, fuel cells, dye-sensitized solar cells (DSSC), hybrid cells, and photovoltaic (PV) devices⁴⁴. Such systems and devices may be suitable for various CleanTech and GreenTech applications.

Large-scale industrial implementation of the photoactive materials can be enabled through manufacture of the materials as coatings and/or thin-film solar panels. Various thin-film coating techniques, such as those discussed above, can be used for industrial-scale engineering of solar panel reactors incorporating the disclosed photoactive materials.

The disclosed photoactive materials can be implemented in conventional photoreactor types. Such photoreactors typically carry out photoactive reactions any various conditions, including various pressures (e.g. pressure 0.001-1000 atm), temperatures (e.g., room temperature−3000° C.) and/or gas-mixture ratios with various flow conditions.

Conventional photoreactor configurations used for large-scale industrial processes include, for example: parabolic trough reactor (PTR)-photoreactors, which may be adapted directly from solar thermal collector designs; compound parabolic collectors (CPC)-photoreactors, which are similar to the PTR photoreactor without using a sun-tracking mechanism, in order to help reduce the cost and complexity of the system; inclined plate collector (IPC)-photoreactors, which is a design including a flat, inclined surface upon which the reactant fluid or gas may flow as a thin film; double skin sheet (DSS)-photoreactor, which is a design which has a relatively long, back and forth convoluted channel on a flat plane, through which the reactant of the suspended photoactive materials flow with the photoactive materials supported on the backing plate; rotating disc photoreactor (RDR) and water bell photoreactors (WBR); optical fibre photoreactors, which is a design having an optical waveguide to channel solar illumination to the photoactive layers contained within; fixed and fluidized bed (FBP) photoreactors and thin film fixed bed photoreactors (TFFBR); and Concentrated Solar Thermal (CST) plant designs, among others⁴⁴.

For all the above-described reactor types, the incorporated photoactive material can be a dynamic, mechanically flexible porous multi-layered metal-oxide embodiment, such as multi-layered porous metal oxide photoactive layers deposited on flexible polymer membranes.

In particular, photon-drive production of fuels at industrial scales may be achieved by incorporating the photoactive materials in a flow-through membrane multi-layer photoreactor, in which gas-permeability through the membrane is controlled through suitable selection of porosity, pore size distribution, permeability and layer selection of the photoactive material. Such a system can be driven by sunlight or CSP. For concentrating and/or focusing light, CSP systems typically use lenses or mirrors and/or tracking systems to focus a relatively large area of sunlight onto a relatively small area. The concentrated light may then be used as heat or as a heat source (e.g. for a conventional power plant to generate solar thermoelectricity) or may be used as high energy source for the disclosed photoactive materials and large-scale photoactive reactions for generating fuels.

The fuels that may be generated include, for example, hydrogen, carbon monoxide, alkanes (such as methane, ethane, propane, isopropane, linear and branched hydrocarbon isomers and possible mixtures thereof), olefins (such as ethylene, propylene, butylene and other linear and branched olefin-isomers and possible mixtures thereof), oxygen-rich hydrocarbon compounds (such as methanol, formaldehyde, ethanol, propanol, formic acid, aldehydes and other oxygenated hydrocarbon compounds) as well as mixtures thereof.

Conventional solar concentrating technologies include, for example: parabolic trough, dish Stirling, concentrating linear Fresnel reflector, solar chimney and the solar power tower configurations, among others.

In an example, the disclosed photoactive materials can be incorporated into PTR-photoreactors and CPC-photoreactors in a CST plant configuration. These systems include a linear parabolic reflector to concentrate light onto a receiver positioned along the reflector's focal line. The receiver is typically a tube, which can be packed with photoactive materials, in the form of flakes, positioned directly above the middle of the parabolic mirror. A gas mixture comprising for example, CO/CO₂ and H₂O and/or CO₂ and various CO/H₂/H₂O mixtures, flows through the packed tube directly from an industrial unit, such as a gas/coal or oil plant and/or any carbon capture and storage (CCS) off-shore and/or on-shore reservoirs. The reflector is able to track the position of the sun over daylight hours. The generated heat from these photoreactors typically lies in the range of about 120-750 degrees C. as the gas-mixture is flowing through the receiver tube and may be then used for large-scale reaction of CO₂ with H₂O and/or various H₂/H₂O mixtures for continuous large-scale generation of fuels.

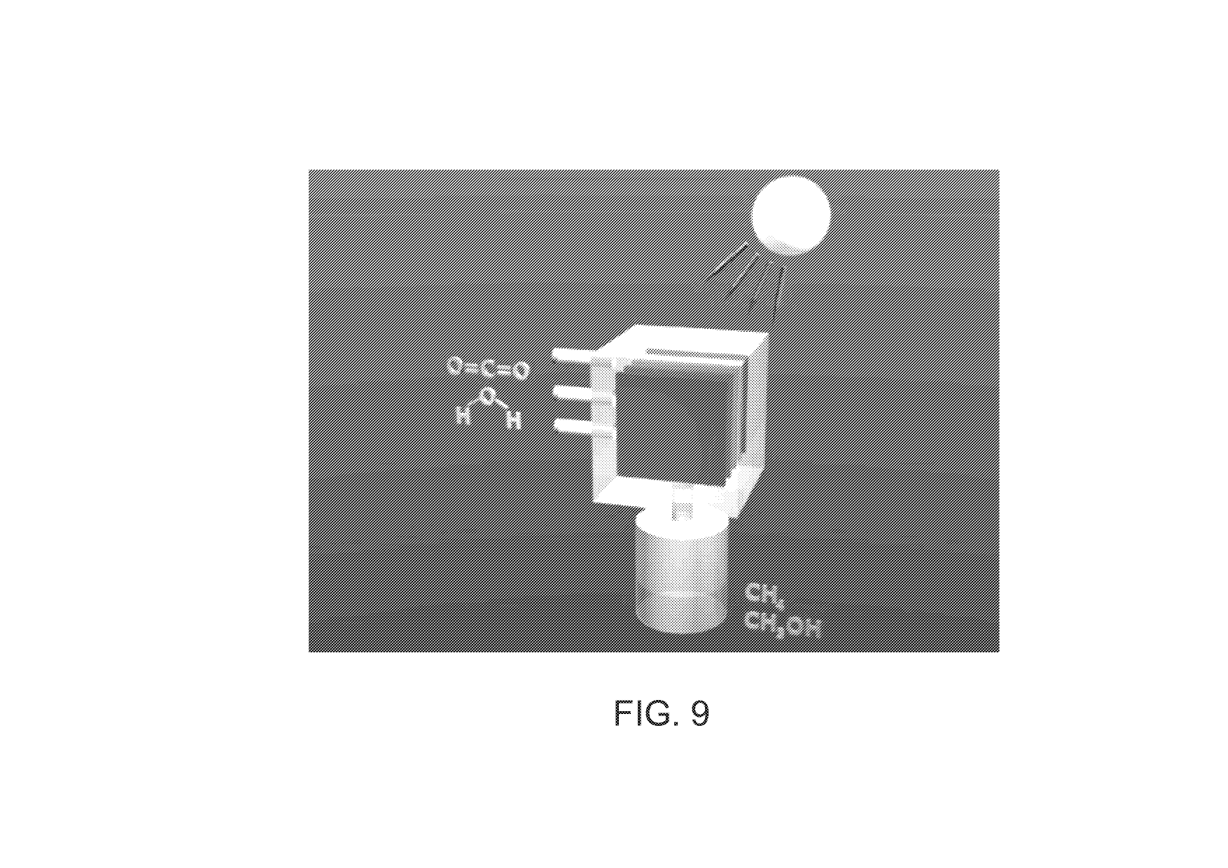

FIG. 9 is a schematic illustration of an example photosynthetic fuel generator having an enclosed array of photoactive materials. In the example of FIG. 9, the apparatus includes a parallel stack of optically transparent photoactive materials in the form of panels, housed inside a transparent reactor chamber. The panels can be contacted with carbon dioxide at a particular pressure, flow rate and/or temperature and a source of hydrogen (e.g., water vapor and/or hydrogen gas), and simultaneously irradiated with sunlight. The fuel (e.g., methane and/or methanol) so generated by the panels may be collected and/or stored in gaseous or liquid form, and/or may be distributed using a conventional fuel network.

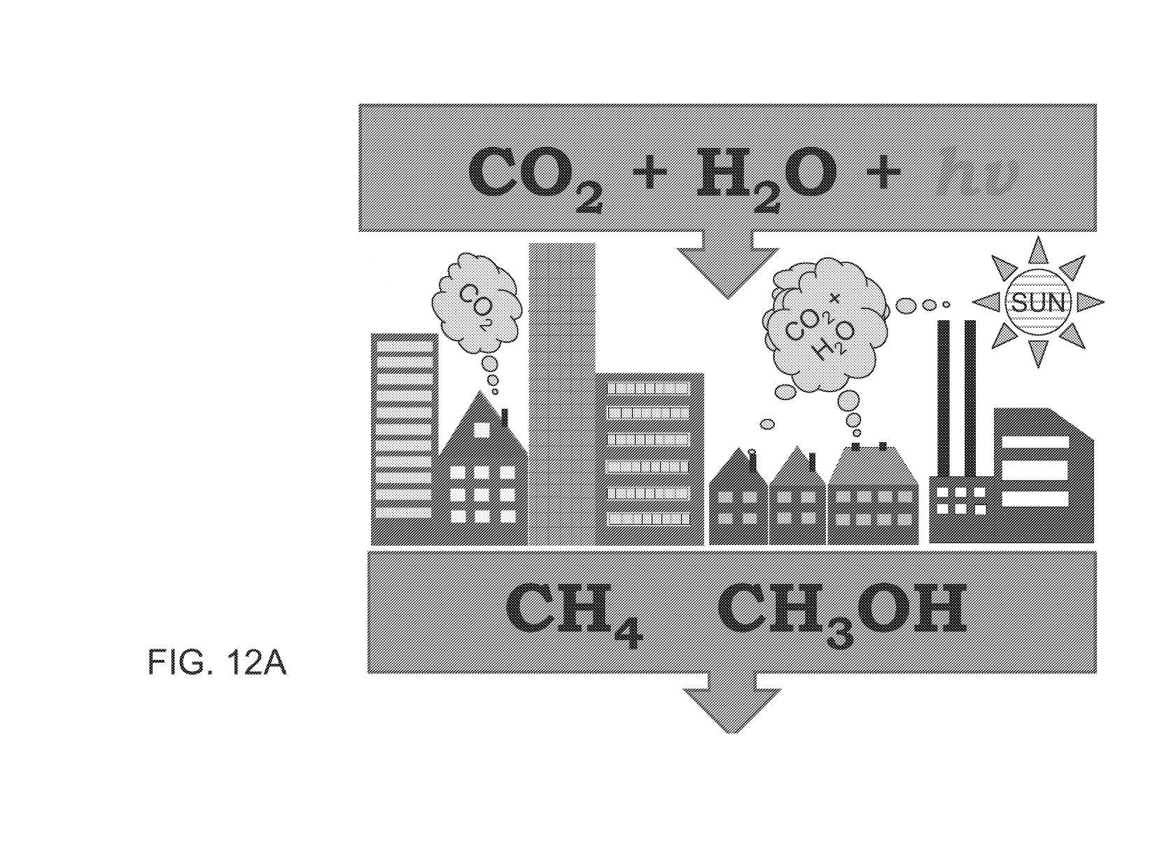

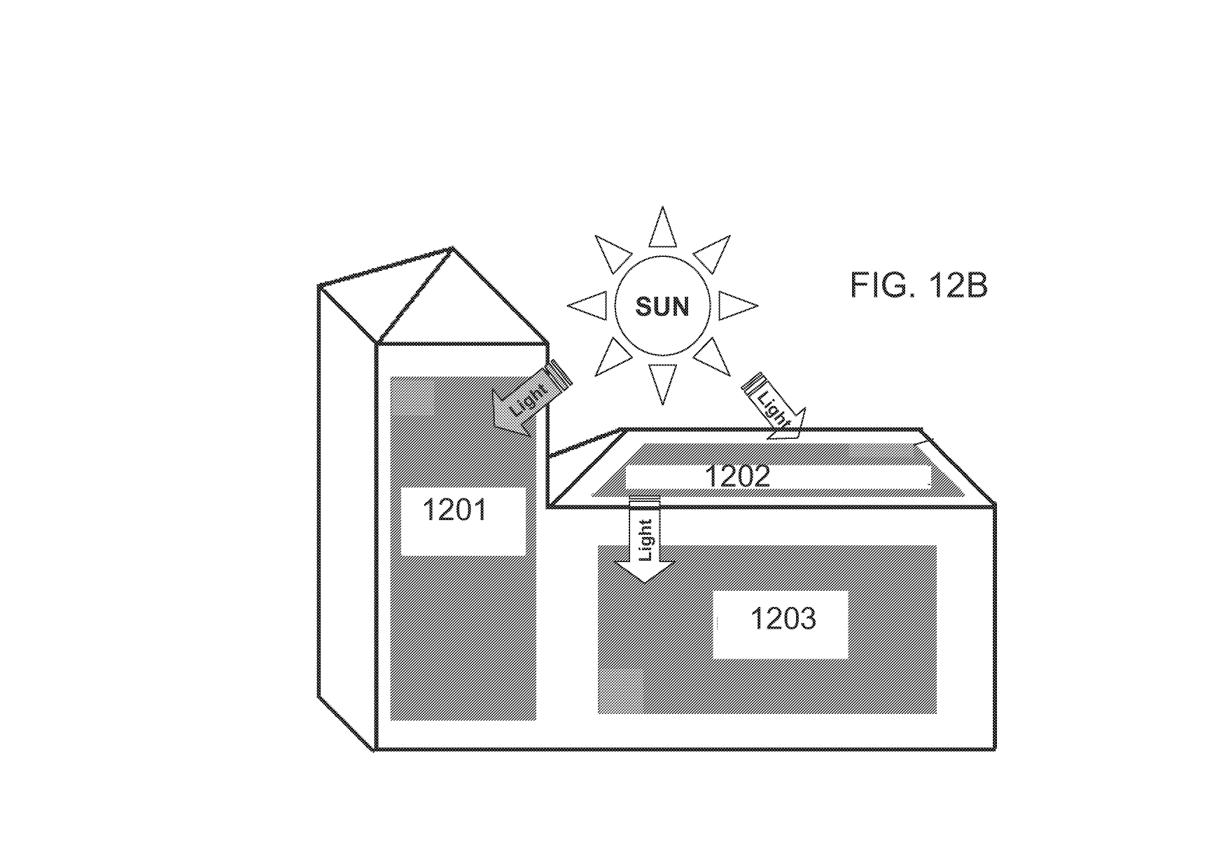

Based on typical solar thermal utility in the United States or Spain, which uses arrays of solar panel reflectors to concentrate sunlight and convert it to heat and through heat exchangers to electricity, solar thermal farms may be organized around a million panels of photoactive materials in a land area of about a square kilometer. Based on these precedents for solar thermal land utilization, a billion such solar panels, membranes and coatings may require about 1000 km² of land. This land usage can reduced substantially by spreading the required area in different sunny open spaces around the world (e.g., placing them on roofs, windows and facades of buildings in villages, towns and cities), as illustrated in FIGS. 12A and 12B.

FIGS. 12A and 12B show examples incorporating the photoactive material in solar panels and solar trees to be used on a utility scale. For example, the photoactive material may be incorporated in personalized energy units, such as in building integrated photosynthetic units (BIPS) in homes and in buildings in cities, villages and urban areas. The photoactive material may be implemented in a building in the form of a solar panel facade 1201, a solar panel roof 1202 and/or a solar panel window 1203.

The disclosed photoactive materials can also be incorporated into solar trees and forests on large-scale solar farmland to produce industrial amounts of fuels through photoactive reactions. By stacking the solar panels one behind the other while maintaining optical transparency throughout the stack, which is facilitated by the high optical transparency of the disclosed photoactive materials, this land requirement may be reduced significantly.

An experimentally determined rate of production of fuels using a panel having an area of about 100 m², incorporating a photoactive material with Fe₂O₃/TiO₂ multi-layers and high optical transparency, is around ≈100-1000 g h⁻¹ m⁻².

When scaled to 10 billion panels, such a rate translates into a rate of conversion of carbon dioxide to fuels of about 10¹⁰ tons/year. This is a globally significant number, as about 10 billion tons of carbon dioxide and other greenhouse gases are currently being deposited into the troposphere every year. This rate of conversion can be enhanced through engineering the structure, composition, nanocrystallinity, surface area and/or porosity of the photoactive material, as discussed above. This rate can be further increased through the use of CSPs.

### Building Integrated Photosynthetic Units

The disclosed photoactive materials can also be manufactured as panels for use on or within buildings. These are referred to as building integrated photosynthetic units (BIPS), as described above, and can be placed on roofs, windows and facades on various buildings in villages, towns and cities, and trees, forests and farms in open land, for example. BIPS can be provided as panels, membranes and coatings for personal or individual photon-driven generation of fuels on a small or large scale.

These fuels may be stored in the house and may be used for heating and cooking, for example, or in a fuel cell to produce electricity for the house and electricity for the car when solar cells cannot.

### H₂O Splitting Applications

Improvements in H₂O splitting (e.g., electro- and/or photocatalytically) as well as catalytic systems with higher conversion rates and more targeted selectivities may be useful, for CO₂ hydrogenation and reforming to become economically feasible and useful on a large scale. In some examples, using solar illumination or CSP irradiation may help to reduce the carbon footprint of the disclosed photoactive materials and fuel generating systems.

Photoelectrolysis⁴⁵ is a process where water (H₂O) is dissociated or split into H₂ and O₂ gas. In an example photo electrochemical cell (PEC), a cell containing an electrolyte (e.g., aqueous, basic neutral or acidic, alcoholic, polar and/or non-polar solvent) may be in contact with a porous photoactive metal oxide or semiconductor single-constituent and/or mixed thin film, or a periodic photonic multi-layered electrode (e.g. made out of TiO₂, WO₃, ZnO, CuO, Cu₂O, CoO, SiC, NiO, Co₃O₄, Fe₃C, MnO₂ or Fe₂O₃ and/or mixed compositions thereof) and, for example, a Pt-counter electrode as well with a reference electrode (e.g., Ag/AgCl). The photon energy for the process required to occur may be ˜1.23 eV. This may be, for example, the energy between the redox levels E^{o}(H₂/H₂O) and E^{o}(O₂/H₂O), e.g. flat-band potentials in the electrolyte. In practical use, the energy required may be higher than this (e.g. 1.4-1.8 V), for example due to over-voltages in the system.

The splitting of water is described by the equation below:

H₂O→H₂ (g)+½O₂ (g)

through the use of a catalyst, such as Fe₂O₃/NiO, Fe₂O₃/Co₃O₄, Co₃O₄/NiO, Co₃O₄/WO₃, Fe₂O₃/MnO₂, Fe₂O₃/CuO, WO₃/MnO₂, Fe₂O₃—MnO₂/WO₃, Fe₂O₃—NiO/Co₃O₄, NiO—MnO₂/Fe₂O₃, CuO—NiO/MnO₂, Fe₂O₃/WO₃, SiC/CUO, Fe₂O₃/Cu₂O, Cu₂O—Fe₂O₃/SiC, NiO—Fe₂O₃/WO₃

and various possible combinations thereof, as provided by a photoactive material.

A consideration for relatively efficient and effective H₂O splitting may be that the bottom of the porous metal oxide electrode conduction band occurs above the E^{o}(H₂/H₂O) level and the top of the valence band of the porous metal oxide electrode occurs below the E^{o}(O₂/H₂O) level. The example porous semiconductor electrode may have an electronic bandgap larger than, for example, 1.23 eV to overcome over-voltages, for example, in order that the generated charge-carriers may be produced by using a relatively large fraction of the solar spectrum.

Various photoactive material arrangements may be coupled together to construct multi-layered junctions in a gradient or tandem configuration to form a conductive electrode, such as a transparent conducting oxide (TCO) electrode. The use of ultra-thin constituent layers in the photoactive material may enable tunneling of electrons through all layers of the photoactive material and may help in avoiding recombination pathways.

Further H₂O-splitting enhancement can be improved by addition/incorporation of SPR materials, as described above, e.g. (Ag, Au, or Cu NPs, as well as alloys e.g. Ag—Au and core@shell e.g. Ag@Au structures thereof) at distinct locations/positions within the photoactive material.

### Anti-Smog and Anti-Pollutant Applications

The disclosed photoactive materials can also be designed to carry out redox reactions for decomposition of organic and/or inorganic pollutants, such as those commonly found in air and/or water. For example, semiconductor nanoparticles, such as TiO₂, are commonly used in purification applications and can be used as a photoactive constituent of the photoactive material.

Conventional anti-smog coatings, such as those employed on roofs, windows and facades in villages, towns and cities, are typically based on a micron thick (typically about 0.1 to 5 μm) TiO₂ layer. However, TiO₂ is strongly absorbing mostly in the UV region of the solar spectrum and therefore harnesses only about 3-5% of sunlight.

In contrast, the disclosed photoactive materials can be designed to be strongly reactive to light in wavelengths more strongly present in sunlight. This may help to enhance the rate of removal of airborne organic and/or inorganic pollutants compared to conventional TiO₂ layers. Conventional treatments applied to anti-smog coatings can be similarly applied to the photoactive materials to provide properties such as super-hydrophilicity, self-cleaning properties, and hydrophobicity, as appropriate.⁴⁶

### Environmental Clean-Up of Organic Pollutants in Air and Water

Another area of application of the photoactive material may be in the removal and/or destruction of contaminants in water treatment or purification.47 Major pollutants in waste water tend to be organic compounds. Small quantities of toxic and precious metal ions or complexes may also be present. Semiconductor nanoparticles, for example TiO2, WO3 or ZnO may provide a system for degrading organic and/or inorganic pollutants in water, through the formation of [—OH] radicals which react with organic and/or inorganic pollutants, through photoreactions.

Many reactions for cleaning environmental pollutants may involve at least the initial process of oxidation of organic molecules by [—OH] radicals generated in photoreactions. Since these photoreactions may proceed in an aqueous suspension of photoactive semiconductor materials or by adsorbing molecules on photoactive semiconductor metal oxide surfaces, water may be initially oxidized by holes generated in photoreactions to form hydroxyl [—OH] radicals. In the subsequent process, [—OH] radicals may react with organic compounds to form oxidized organic species or decomposed organic products. This process may be referred to as indirect oxidation. These processes may also be used in air-purification processes.

Water treatment based on photoreactions may provide an alternative to other advanced oxidation technologies (e.g., UV-H₂O₂ and UV-O₃), such as those designed for environmental remediation by oxidative mineralization. The photon-driven mineralization of organic compounds in aqueous media may proceed through the formation of a series of intermediates of progressively higher oxygen to carbon ratios. For example, photon-driven degradation of phenols may yield hydroquinone, catechol, and benzoquinone as the major intermediates that may be oxidized to carbon dioxide and water.

Gas-solid heterogeneous photon-driven oxidations of vapour or gas phase contaminants may also be useful. These reactions may be useful for applications in air purification. The photoreaction rates of some compounds, for example, trichloroethylene may be orders of magnitude faster in the gas phase than in aqueous solution. These high reaction rates may be useful in such reactions for air or other gas or vapour purifications, for example.

Gas-solid photon-driven oxidation for remediation of contaminants in gas streams may be applied to treating organic compounds, for example including alkenes, alkanes, aromatics, olefins, ketones, aldehydes, alcohols, aliphatic carboxylic acids and halogenated hydrocarbons, among others. Semiconductors (e.g., TiO₂, ZnO or Fe₂O₃) may exhibit useful photoactivity for these applications. In general, the reaction rates in gas-solid photoreactors may be much higher than those reported for liquid-solid photoreactors; for example efficiencies higher than 100% may be possible for some gas-phase photon-driven oxidations. The photoactivity in such gas-solid heterogeneous systems may be influenced by the presence of water vapour and reaction temperature, for example.

### Comparison to Conventional Powders

The disclosed photoactive materials are expected to exhibit superior activity compared to conventional powder form photoactive materials.

FIGS. 6A and 6B are schematic diagrams comparing a conventional photoactive powder (FIG. 6B) with an example of the multi-layered photoactive material of the present disclosure (FIG. 6A). As shown, in the multi-layered photoactive material, photoactive constituents A and B are formed into separate porous nanoparticle layers. In the conventional powder form, the photoactive constituents are randomly jumbled together.

In the design of conventional photochemical reactors, photoactive powders (e.g. powdered Fe₂O₃, TiO₂) is typically immobilized (e.g., on various solid supports, substrates, membrane and/or various panel architectures, among others) so that its recovery and reuse may be facilitated. However, problems of efficient light transmission, scattering, reflection and utilization within conventional photochemical powder-reactors limit the use of this technology for large-scale application.

In contrast, the disclosed photoactive material, by providing high optical transparency, allows for high photon penetration, thereby allowing light to potentially access every photoactive site, resulting in greater efficiency.

While conventional heterogeneous metal oxide photoactive powder forms of materials have been documented to be able to photochemically reduce carbon dioxide and oxidize water and/or hydrogen to methane or methanol, their conversion efficiency is typically too low for the practical large-scale production of fuels and remediation of carbon dioxide and other greenhouse gases. Also, their fine powder form are not conducive to the efficient absorption of light by the photoactive material, due to light losses through the deleterious light scattering and reflection of the powder form, resulting in small photon penetration depth and hence poor response to incident light.

Moreover, the powder format may not be practical or safe for engineering industrial scale photoactive reactors.

The single-layer mixed photoactive material is also distinct from simply a thin layer of the conventional powder. The single-layer photoactive material has distinct packing and particle arrangements due to the colloidal charge effects. The disclosed photoactive material provides porous photoactive layers having much smaller photoactive constituent nanoparticles (e.g., 3-15 nm in diameter) and with higher surface area and porosity than conventional powder photocatalysts (which have particle sizes typically in the range of about 30-100 nm)

Further, while the photoactive material has been described as being manufacturable as a thin layer (e.g., no more than 1000 nm thick), such a thin layer cannot be achieved using conventional powders, which typically produce coatings that are several microns thick.

The disclosed photoactive material provide advantages that are useful for its incorporation into solar panels, membranes, coatings and various photoreactor designs, compared to conventional photoactive powders. Such advantages include high optical transparency of the disclosed photoactive material compared to conventional powders. This high optical transparency helps to reduce or minimize reflection and scattering light losses and helps to increase or maximize the penetration of light throughout the entire thickness of a panel, membrane or coating incorporating the photoactive material. This allows incident light to access all or most possible photoactive sites within the material, leading to relatively high quantum yields, enhancing the generation of chemically reactive electrons and holes, resulting in more redox reactions resulting in fuels from carbon dioxide. The incorporation of reflecting and/or scattering layers into such optically transparent panels further enhances the efficiency of these light-driven processes. Furthermore, optical transparency allows one panel to be stacked behind the other to provide even higher efficiency.

Conventional photoactive powders also typically have poor charge generation and separation, due to their relatively large particle size relative to the wavelength of light. This results in poor charge carrier separation and redox reactivity and resulting therefore in overall lower photoactive efficiency.

The disclosed photoactive material may be used for CO₂ to natural gas (e.g., CH₄) gas-solid heterogeneous light/sunlight or CSP driven reduction or photocatalytic reforming processes. In The gas-solid heterogeneous CO₂ reduction may be performed under batch or different flow-through conditions in various photoreactor designs. The gas-solid heterogeneous CO₂ reduction may be performed under different reaction temperatures, e.g., at room temperature (RT) or higher. The gas-solid heterogeneous CO₂ reduction may be performed under different pressure conditions, e.g., at about 0.01 psi or higher. The gas-solid heterogeneous CO₂ reduction may be perform with different light sources (e.g., with or without a cut-off filter), as well as at broad spectrum or specific wavelengths (e.g., by using different monochromatic light). The gas-solid heterogeneous CO₂ reduction may be performed under natural sunlight or under 1.5 AM conditions (e.g., by using simulated sunlight and temperature conditions).

The disclosed photoactive material may be used for broad and large scale industrial and/or various cleantech applications. For example, the photoactive material may be useful for purification and cleaning of environmental pollutants (e.g. halogenated hydrocarbons, nitric oxides, green houses gases) from air and/or water. The photoactive material may be useful for broad petrochemical catalytic applications, including: petroleum refining, naphtha reforming, hydrotreating, cracking, hydrocracking, isomerization, and alkylation processes, among others. As discussed herein, the photoactive material may be useful for relatively large scale CO₂ reforming processes to fuels. Further, the photoactive material may be useful for conversion of syngas (CO/H₂) and industrial water-gas shift processes. The photoactive material may be useful for industrial large scale methanation and methanol synthesis processes and Fischer-Tropsch synthesis (FTS).

The embodiments of the present disclosure described above are intended to be examples only. Alterations, modifications and variations to the disclosure may be made without departing from the intended scope of the present disclosure. In particular, selected features from one or more of the above-described embodiments may be combined to create alternative embodiments not explicitly described. All values and sub-ranges within disclosed ranges are also disclosed. The subject matter described herein intends to cover and embrace all suitable changes in technology. All references mentioned are hereby incorporated by reference in their entirety.

### REFERENCES

¹ a) N. S. Lewis, Powering the Planet, MRS Bulletin, 2007, 32, 808-820. b) N. Armaroli, V, Balzani, Energy for a Sustainable World, Wiley-VCH, Germany, 2011, pp. 1-352.
² A. F. Collings, C. Critchley, Artificial Photosynthesis, Wiley-VCH, 2005, Germany, pp. 13-34.
³ a) K. S. Gould, D. W. Lee, American Journal of Botany, 1996, 83, 45-50. b) R. M. Graham, D. W. Lee, K. Norstog, American Journal of Botany, 1993, 80, 198-203. c) H. Zhou, X. F. Li, T. X. Fan, F. E. Osterloh, J. Ding, E. M. Sabio, D. Zhang, Q. X. Guo, Adv. Mater. 2010, 22, 951-957.
⁴ a) J. L. G. Fierro, Metal Oxides Chemistry and Applications, CRC Press, Taylor & Francis Group, 2006, USA, pp. 1-765. b) J. A. Rodríguez, M. Fernández-García, Synthesis, Properties, and Applications of Oxide Materials, Wiley-Interscience, USA, 2007, pp. 1-717 c) V. E. Heinrich, P. A. Cox, The Surface Science of Metal Oxides, Cambridge University Press, 2000, USA, pp. 1-458.
⁵ H. Goesmann, C. Feldmann, Angew. Chem., Int. Ed. 2010, 49, 1362-1395.
⁶ H. Zhou, T. Fan, D. Zhang, Chem. Cat. Chem. 2011, 3, 513-528.
⁷ a) A. J. Bard, J. Photochem, 1979, 10, 59-75. b) H. Zhou, T. Fan, D. Zhang, Chem. Cat. Chem. 2011, 3, 513-528.
⁸ O. L. Stroyuk, S. Y. Kuchmiy, A. I. Kryukov, V. D. Pokhodenko, Semiconductor Catalysis and Photocatalysis on the Nanoscale, Nova Science Publishers, Inc., USA, 2010, pp. 1-183.
⁹ a) A. Weibel, R. Bouchet, F. Boulc'h, P. Knauth, Chem. Mater. 2005, 17, 2378-2385. b) V. M. Gun'ko, V. I. Zarko, R. Leboda, E. Chibowski, Advances in Colloid and Interface Science, 2001, 91, 1-112.
¹⁰ a) S. Lowell, J. E. Shields, M. A. Thomas, M. Thommes, Characterization of Porous Materials and Powders: Surface Area, Pore Size and Density (Particle Technology Series), Springer-Verlag, 2006, 2nd Ed., Germany, pp. 1-347. b) D. Dollimore, P. Spooner, A. Turner, Surface Technology, 1976, 4, 121-160.
¹¹ a) J. I. Langford, D. Louër Powder Diffraction Rep. Prog. Phys. 1996, 59, 131-234. b) T. Ungár, J. Gubicza Z. Kristallogr. 2007, 222, 114-128.
¹² R. J. Hunter, Zeta Potential in Colloid Science: Principles and Applications; Academic Press: London, UK, 1988; pp 24-54.
¹³ H Zhang, G. Chen, D. W. Bahnemann, J. Mater. Chem. 2009, 19:5089-5121.
¹⁴ http://refractiveindex.info/
¹⁵ Y. Qu, H. Zhou, X. Duan, Nanoscale, 2011 (in revision) DOI: 10.1039/c1nr10668f
¹⁶ a) Y. Lin, S. Zhou, X. Liu, S. W. Sheehan, D. Wang, J. Amer. Chem. Soc., 2009, 131, 2772-2773. b) Y. Lin, S. Zhou, S. W. Sheehan, D. Wang, J. Amer. Chem. Soc., 2011, 133, 2398-2401.
¹⁷ D. Friedmann, H. Hansing and D. Bahnemann, Z. Phys. Chemie Int. J. Res. Phys. Chem. Chem. Phys. 2007, 221, 329-348.
¹⁸ E. Redel, P. Mirchev, C. Huai, S. Petrov, G. A. Ozin, ACS Nano, 2011, 5, 2861-2869.
¹⁹ a) A. C. Arsenault, T. J. Clark, G. von Freymann, L. Cademartiri, R. Sapienza, J. Bertolotti, E. Vekris, S. Wong, V. Kitaev, I. Manners, R. Z. Wang, S. John, D. S. Wiersma, G. A. Ozin, Nature Materials 2006, 5, 179-184. b) D. P. Puzzo, A. C. Arsenault, I. Manners, G. A. Ozin, Angew. Chem., Int. Ed. 2009, 48, 943. c) L. D. Bonifacio, B. V. Lotsch, D. P. Puzzo, F. Scotognella, G. A. Ozin, Adv. Mater. 2009, 21, 1641.
²⁰ Slow Light, Nature Photonics, Focus Issue, 2008, 2, 447-509.
²¹ a) J. I. L. Chen, G. A. Ozin, J. Mater. Chem. 2009, 19, 2675-2678. b) J. I. L. Chen, G. von Freymann, S. Y. Choi, V. Kitaev, G. A. Ozin, J. Mater. Chem. 2008, 18, 369. c) J. I. L. Chen, G. von Freymann, V. Kitaev, G. A. Ozin, J. Am. Chem. Soc. 2007, 129, 1196.
²² a) S, John, Localization of Light and the Photonic Band Gap Concept, Springer-Verlag, 2005, Germany, pp. 1-300. b) J. D. Joannopoulos, S. G. Johnson, J. N. Winn, R. D. Meade, Photonic Crystals—Molding the Flow of Light, 2nd. Ed., Princeton University Press, 2008.
²³ I. Thomann, B. A. Pinaud, Z. Chen, B. M. Clemens, T. F. Jaramillo, M. L. Brongersma, Nano Lett. 2011, 11, 3440-3446.
²⁴ a) P. G. O'Brien, N. P. Kherani, A. Chutinan, G. A. Ozin, S. John, S. Zukotynski, Adv. Mater. 2008, 20, 1577-1582. b) P. G. O'Brien, A. Chutinan, K. Leong, N. P. Kherani, G. A. Ozin, S. Zukotynski, Optics Express 2010, 18, 4478-4490.
²⁵ J. S. White, G. Veronis, Z. Yu, E. S. Barnard, A. Chandran, S. Fan, M. L. Borngersma, Optical Letters, 2009, 34, 686-688.
²⁶ E. Redel, S. Petrov, G. Omer, J. Moir, C. Huai, P. Mirtchev, G. A. Ozin, Small 2011, (in revision) DOI: 10.1002/sm11.201101596
²⁷ a) M. Niederberger, N. Pinna, Metal Oxide Nanoparticles in Organic Solvents; Springer-Verlag: London, 2009, pp. 1-209. b) H. Goesmann, C. Feldmann, Angew. Chem., Int. Ed. 2010, 49, 1362-1395. c) 3) M. Niederberger, Acc. Chem. Res. 2007, 40, 793-800. d) N. Pinna, M. Niederberger, Angew. Chem., Int. Ed. 2008, 47, 5292-5304. e) Brinker, C. J.; Scherer, G. W. Sol-Gel Science: The Physics and Chemistry of Sol-Gel Processing; Academic Press: London, UK, 1990; pp 1-909.
²⁸ M. C. Jeong, B.-Y. Oh, O.-H. Nam, T. Kim, J.-M. Myoung, Nanotechnolgy, 2006, 17, 526-530.
²⁹ C. J. Sartoretti, B. D. Alexander, R. Solarska, J. Phys. Chem. B, 2005, 109, 13685-13692.
³⁰ A. Duret, M. Grätzel, J. Phys. Chem. B, 2005, 109, 17184-17191.
³¹ G. B. Smith, C. G. Granqvist, Green Nanotechnology, CRC Press, 2011, 413-427.
³² J. A. Glasscock, P. R. F. Barnes, I. C. Plumb, N. Savvides, J. Phys. Chem. C, 2007, 111, 16477-16488.
³³ A. Kay, I. Cesar, M. Grätzel, J. Amer. Chem. Soc., 2008, 128, 15714-15721.
³⁴H. E. Prakasam, O.K. Vargehese, M. Paulose, C. A. Grimes, Nanotechnology, 2006, 17, 4285-4291.
³⁵ Y.-S. Hu, A. K. Shwarsctein, A. J. Forman, D. Hazen, J.-N. Park, E. W. McFarland, Chem. Mater., 2008, 20, 3803-3805.
³⁶ E. Yablonovitch, J. Opt. Soc. Am., 1982, 72, 899-907.
³⁷M. Grundmann, The Physics of Semiconductors, Springer-Verlag, 2006, Germany, pp. 474-576.
³⁸ a) P. G. O'Brien, N. P. Kherani, A. Chutinan, G. A. Ozin, S. John, S. Zukotynski, Adv. Mater. 2008, 20, 1577-1582. b) P. G. O'Brien, A. Chutinan, K. Leong, N. P. Kherani, G. A. Ozin, S. Zukotynski, Optics Express 2010, 18, 4478-4490.
³⁹M. W. Abee, S.C. York, D. F. Cox, J. Phys. Chem. B, 2001, 105, 7755-7761.
⁴⁰ a) G. Boschloo, A. Hagfeldt J. Phys. Chem. B 2001, 105, 3039-3044. b) D. Barreca, C. Massignan, S. Daolio, M. Fabrizio, C. Piccirillo, L. Armaleo, E. Tondello Chem. Mater. 2001, 13, 588-593
⁴¹ U. Kreibig, M. Vollmer, Optical Properties of Metal Clusters, Springer Series in Materials Science 25, Springer-Verlag, Germany, 1995, pp. 1-526.
⁴² J. R. Evans, H. Poorter, Plant, Cell and Environment 2001, 24, 755-767.
⁴³ R. J. Braham, A. T. Harris, Ind. Eng. Chem. Res., 2009, 48, 8890-8905.
⁴⁴ a) M. Graetzel, Photoelectrochemical cells, Nature, 2001, 414, 338-344. b) M. Graetzel, Perspectives for Dye-Sensitized Nanocrystalline Solar Cells. Prog. Photovoltaic Res. Applic. 2000, 8, 171-185. c) B. O'Regan, M. Graetzel, Nature, 1991, 353, 737-740.
⁴⁵ V. R. Satsangi, S. Dass, R. Shrivastav, On Solar Hydrogen & Nanotechnology (Eds.: Vayssieres, L.); John Wiley & Sons (Asia): Singapore 2009; pp 349-397.
⁴⁶ S. Ramanathan, Thin Film Metal Oxides, Springer Science Business Media, 2010, pp. 255-328.
⁴⁷ a) M. Kaneko, I. Okura, Photocatalysis Science and Technology, Springer-Verlag & Kodansha Ltd. 2002, Japan, pp. 1-346. b) N. Serpone, E. Pelizzetti, Photocatalysis Fundamentals and Applications, John Wiley & Sons, Inc. 1989, USA, pp. 1-637.

1. A photoactive material comprising:
nanoparticles of at least one first photoactive constituent; and
nanoparticles of at least one second photoactive constituent;
the at least one first and second constituents each being selected to have respective conduction band energies, valence band energies and electronic band gap energies, to enable photon-driven generation and separation of charge carriers in each of the at least one first and second constituents by absorption of light in the solar spectrum;
the nanoparticles of each of the at least one first and second constituents being mixed together to form a layer;
the nanoparticles of each of the at least one first and second constituents having diameters smaller than wavelengths of light in the solar spectrum, to provide optical transparency for absorption of light; and
wherein the charge carriers, upon photoactivation, are able to participate in redox reactions occurring in the photoactive material.

2. A photoactive material comprising:
nanoparticles of at least one first photoactive constituent; and
nanoparticles of at least one second photoactive constituent;
the at least one first and second constituents each being selected to have respective conduction band energies, valence band energies and electronic band gap energies, to enable photon-driven generation and separation of charge carriers in each of the at least one first and second constituents by absorption of light in the solar spectrum;
wherein the nanoparticles of the at least one first constituent form at least one first layer and the nanoparticles of the at least one second constituent form at least one second layer;
the nanoparticles of each of the at least one first and second constituents having diameters smaller than wavelengths of light in the solar spectrum, to provide optical transparency for absorption of light;
wherein the photoactive material comprises the at least one first layer and the at least one second layer in an alternating layer arrangement; and
wherein the charge carriers, upon photoactivation, are able to participate in redox reactions occurring in the photoactive material.

3. The photoactive material of claim 1 wherein the conduction band and valence band energies of the at least one first constituent are higher than those of the at least one second constituent, to enable the photon-driven generation and separation of charge carriers.

4. The photoactive material of claim 1 wherein the photon-driven generation and separation of charge carriers is enabled by absorption of light in the visible spectrum.

5. The photoactive material of claim 1 wherein at least one layer of the photoactive material is porous, to permit permeation by reactants and collection of products of the redox reactions.

6. The photoactive material of claim 5 wherein the at least one porous layer has a porosity in the range of about 10% to about 90% by volume.

7.-9. (canceled)

10. The photoactive material of claim 2 wherein the respective layer thicknesses of each of the at least one first and second layers matches the exciton diffusion lengths of each of the at least one first and second constituents, respectively.

11. The photoactive material of claim 1 wherein the nanoparticles of each of the at least one first and second constituents have respective diameters substantially equal to the exciton diffusion lengths of each of the at least one first and second constituents, respectively.

12. The photoactive material of claim 1 wherein each layer has a thickness in the range of about 1 nm to about 1000 nm.

13. (canceled)

14. The photoactive material of claim 1 wherein the nanoparticles of the at least one first and second constituents are selected to have sizes dependent on selection of the at least one first and second constituents, respectively.

15. The photoactive material of claim 1 wherein the nanoparticles of the at least one first and second constituents have diameters in the range of about 1 nm to about 50 nm.

16. (canceled)

17. The photoactive material of claim 1 wherein the nanoparticles of the at least one first and second constituents have a geometry selected from the group consisting of: a nanosphere; a nanopolyhedron; a nanowire; a nanorod; a nanosheet and a random geometry.

18. The photoactive material of claim 1 wherein the at least one first and second constituents are selected from the group consisting of: metal oxides, metal carbides, metal borides, metal chalcogenides, metal pnictides, metal silicides, and metal oxyhalides.

19. The photoactive material of claim 18 wherein the metal oxide is selected from the group consisting of: simple metal oxides, mixed metal oxides, doped metal oxides and multicomponent mixed metal oxides.

20. The photoactive material of claim 18 wherein the at least one first constituent and the at least one second constituent are selected from the following pairings X/Y, where X is the first constituent and Y is the second constituent: Fe₂O₃/TiO₂; Fe₂O₃/WO₃; ZnO/TiO₂; ZnO/WO₃; CuO/Fe₂O₃; CuO—ZnO/Fe₂O₃; CuO/TiO₂; CuO/WO₃; CuO—ZnO/Ti O₂; CuO—ZnO/WO₃; CuO—Fe₂O₃/ZnO; CoO/TiO₂; Co₃O₄/WO₃; Co₃O₄—ZnO/TiO₂; Co₃O₄—Fe₂O₃/WO₃; CuO—Co₃O₄/Fe₂O₃; CeO₂/Fe₂O₃; CeO₂/TiO₂; CeO₂/WO₃; CeO₂—NiO/TiO₂; COO—CeO₂/WO₃; ATO/Fe₂O₃; Fe₂O₃/NiO—CO₃O₄; Cu₂O-ATO/Fe₂O₃; NiO/Fe₂O₃; NiO/TiO₂; SiC/CuO; ITO/WO₃; CU₂O/Fe₂O₃; Cu₂O/TiO₂; Fe₂O₃/NiO; ATO-CuO/SiC; NiO—Fe₂O₃/Cu₂O; SiC/Cu₂O; SiC—Cu₂O/Fe₂O₃; TiO₂/WO₃; ITO/Cu₂O; Fe₂O₃—CuO/NiO; Fe₂O₃—NiO/CuO; ZnFe₂O₄/TiO₂; MgCo₂O₄/WO₃; TiO₂/ATO; Fe₂O₃—CuO/ATO; BiVO₄/NiO; Bi₂WO₆/Cu₂O; NjWO₄/Fe₂O₃—CU₂O; ITO-Cu₂O/SiC; Fe₂O₃/Co₃O₄; CO₃O₄/NiO; CO₃O₄/WO₃; Fe₂O₃/MnO₂; WO₃/MnO₂; Fe₂O₃—MnO₂/WO₃; Fe₂O₃—NiO/Co₃O₄; NiO—MnO₂/Fe₂O₃; CuO—NiO/MnO₂; Cu₂O—Fe₂O₃/SiC; and NiO—Fe₂O₃/WO₃.

21. The photoactive material of claim 1 wherein the at least one first and second constituents is a semiconductor material.

22. The photoactive material of claim 2 wherein the alternating layer arrangement is periodic;
the at least one first and second layers having at least one of: a refractive index contrast; a difference in layer thicknesses; and a difference in porosities;
wherein the at least one of: a refractive index contrast, a difference in layer thicknesses, and a difference in porosities gives rise to a photonic stop band; and
wherein slow photon effects occur in given wavelengths at the edges of the photonic stop band, and the slow photon effects promote absorption of light at the given wavelengths.

23. The photoactive material of claim 1 further comprising plasmonic nanoparticles embedded in at least one layer for amplifying the absorption of light.

24. The photoactive material of claim 1 further comprising up-converter particles embedded in at least one layer for converting wavelengths of incident light from a range outside the visible spectrum to a range at least partially overlapping with the visible spectrum.

25. The photoactive material of claim 2 wherein layer thicknesses in the layers of the alternating arrangement gradually increase or decrease.

26. The photoactive material of claim 1 wherein the photoactive material is in the form of a film, a powder, flakes, a dispersion or a coating.

27. (canceled)

28. The photoactive material of claim 1 further comprising a substrate for supporting the photoactive material.

29. The photoactive material of claim 28 wherein the substrate is selected from the group consisting of: a non-porous substrate, a porous substrate, a flexible substrate and an inflexible substrate.

30. A photoactive material assembly comprising:
at least one first photoactive material according to claim 1 superimposed with at least one second photoactive material according to claim 1.

31. A photoreactor comprising a photoactive panel, membrane or tube incorporating the photoactive material of claim 1.

32. A method for generating a fuel by redox reactions of carbon dioxide and at least one of water and hydrogen, using the photoactive material of claim 1.

33. (canceled)
